(19) United States
(12) Patent Application Publication
(10) Pub. No.: US 2022/0193209 A1
(43) Pub. Date: Jun. 23, 2022

(54) COMPOSITIONS AND METHODS FOR REACTIVATING LATENT IMMUNODEFICIENCY VIRUS AND/OR TREATING IMMUNODEFICIENCY VIRUS INFECTION

(71) Applicant: The J. David Gladstone institutes, a testamentary trust established under the Will of j. David Glads, San Francisco, CA (US)

(72) Inventors: Albert Vallejo Gracia, San Francisco, CA (US); Emilie Besnard, San Francisco, CA (US); Melanie Ott, Mill Valley, CA (US); Eric Verdin, San Francisco, CA (US)

(21) Appl. No.: 17/424,453

(22) PCT Filed: Jan. 16, 2020
(86) PCT No.: PCT/US2020/013920

§ 371 Date: Jul. 20, 2021

Related U.S. Application Data
(60) Provisional application No. 62/794,850, filed on Jan. 21, 2019.

(51) Int. Cl. A61K 38/55; A61K 45/06; A61P 31/18

(52) U.S. Cl. CPC A61K 38/55; A61P 31/18; A61K 45/06

(57) ABSTRACT

The present disclosure provides compositions and methods for reactivating latent immunodeficiency virus. The present disclosure provides compositions and methods for treating an immunodeficiency virus infection.

### STATEMENT REGARDING FEDERALLY SPONSORED RESEARCH

This invention was made with government support under Grant No. 1DP1DA038043 awarded by the National Institutes of Health. The government has certain rights in the invention.

### INTRODUCTION

The major barrier to eradicate the Human Immunodeficiency Virus (HIV-1) from infected patients is the persistence of latently infected cells, primarily memory CD4⁺ T cells. Memory CD4⁺ T cells are rare, long-lived, and generally quiescent. Although antiretroviral therapy (ART) effectively suppresses HIV replication, therapy interruption results in reactivation of the latent reservoir, necessitating life-long treatment, and there is no cure. One strategy to eliminate latency after its formation is to activate virus production using latency reversing agents (LRAs) with the objective of triggering cell death through virus-induced cytolysis or immune clearance. Efficient viral reactivation requires a calibrated degree of activation that avoids inducing polyclonal T cell activation or a cytokine storm that would negate the potential benefit. Known LRAs target various pathways to induce viral reactivation, and include PKC agonists (e.g. Prostratin), epigenetic modulators (e.g. HDAC or bromodomain inhibitors), and modulators of the PI3K or mTOR signaling pathways. However, to date, clinical trials of existing LRAs have not demonstrated successful reduction of the latent reservoir. Accordingly, there is a need in the art for new latency reversing agents and methods of using same. The present disclosure addresses this need and provides related advantages.

### LITERATURE

Trinité, B. et al. Suppression of Foxo1 activity and down-modulation of CD62L (L-selectin) in HIV-1 infected resting CD4 T cells. PLoS One 9, (2014); Carrette et al. FOXO1, T-cell trafficking and immune responses. Adv Exp Med Biol, 665: 3-16.

### SUMMARY OF THE INVENTION

The present disclosure provides compositions and methods for reactivating latent immunodeficiency virus. The present disclosure also provides compositions and methods for treating an immunodeficiency virus infection.

The present disclosure provides a method of reactivating latent human immunodeficiency virus (HIV) integrated into the genome of a cell infected with HIV, the method comprising contacting the cell with a FOXO1 inhibitor that reactivates latent HIV integrated into the genome of the cell, wherein the contacting takes place in the absence of an exogenously supplied immunodeficiency virus immunogen. In some cases, the FOXO1 is a polypeptide comprising an amino acid sequence having at least 95%, at least 98%, at least 99%, or 100%, amino acid sequence identity to the human FOXO1amino acid sequence set forth in FIG. 11.

The present disclosure further provides a method of reactivating latent human immunodeficiency virus (HIV) integrated into the genome of a cell infected with HIV, the method comprising contacting the cell with an endoplasmic reticulum (ER) stress-inducing agent that reactivates latent HIV integrated into the genome of the cell. In some such embodiments, the contacting takes place in the absence of an exogenously supplied immunodeficiency virus immunogen. An ER stress-inducing agent may include, but is not limited to, fenretinide and bortezomib. In some embodiments, the contacting comprises contacting the cell with or without a Ca²⁺-mobilizing agent such as ionomycin.

The present disclosure further provides a method of reducing the number of cells containing a latent human immunodeficiency virus in an individual, the method comprising administering to the individual an effective amount of a FOXO1 inhibitor that reactivates latent HIV integrated into the genome of one or more cells in the individual, wherein the administering does not comprise administering an immunodeficiency virus immunogen to the individual.

The present disclosure further provides a method of reducing the number of cells containing a latent human immunodeficiency virus in an individual, the method comprising administering to the individual an effective amount of an endoplasmic reticulum (ER) stress-inducing agent, e.g., fenretinide or bortezomib, that reactivates latent HIV integrated into the genome of one or more cells in the individual. In some such embodiments, the administering does not comprise administering an immunodeficiency virus immunogen to the individual. In some embodiments, the administering comprises administering a Ca²⁺-mobilizing agent such as ionomycin.

The present disclosure further provides a method of treating a human immunodeficiency virus (HIV) infection in an individual, the method comprising: administering to the individual an effective amount of a first active agent, wherein the first active agent is a FOXO1 inhibitor that reactivates latent HIV integrated into the genome of a cell in the individual; and administering to the individual an effective amount of a second active agent, wherein the second active agent inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity, and wherein the administering to the individual of an effective amount of a first active agent does not comprise administering an immunodeficiency virus immunogen to the individual.

The present disclosure further provides a method of treating a human immunodeficiency virus (HIV) infection in an individual, the method comprising: administering to the individual an effective amount of a first active agent, wherein the first active agent is an endoplasmic reticulum (ER) stress-inducing agent, e.g., fenretinide or bortezomib, that reactivates latent HIV integrated into the genome of a cell in the individual; and administering to the individual an effective amount of a second active agent, wherein the second active agent inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity. In some such embodiments, the administering to the individual of an effective amount of a first active agent does not comprise administering an immunodeficiency virus immunogen to the individual. In some embodiments, the administering comprises administering a Ca²⁺-mobilizing agent such as ionomycin.

The present disclosure further provides a method of treating a human immunodeficiency virus (HIV) infection in an individual, the method comprising: administering to the individual an effective amount of a first active agent, wherein the first active agent is a FOXO1 inhibitor that reactivates latent HIV integrated into the genome of a cell in the individual; and administering to the individual an effective amount of an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof, and wherein the administering to the individual of an effective amount of a first active agent does not comprise administering an immunodeficiency virus immunogen to the individual.

The present disclosure further provides a method of treating a human immunodeficiency virus (HIV) infection in an individual, the method comprising: administering to the individual an effective amount of a first active agent, wherein the first active agent is an endoplasmic reticulum (ER) stress-inducing agent, e.g., fenretinide or bortezomib, that reactivates latent HIV integrated into the genome of a cell in the individual; and administering to the individual an effective amount of an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof. In some such embodiments, the administering to the individual of an effective amount of a first active agent does not comprise administering an immunodeficiency virus immunogen to the individual. In some embodiments, the administering comprises administering a Ca²⁺-mobilizing agent such as ionomycin.

The present disclosure further provides a method of treating a human immunodeficiency virus (HIV) infection in an individual, the method comprising: administering to the individual an effective amount of an active agent, wherein the active agent is an activator of FOXO1 or an inhibitor of endoplasmic reticulum (ER) stress, that suppresses establishment and/or maintenance of HIV latency in a cell of the individual. Such a method may be suitable for use early in infection, e.g., within 24, 48, or 72 hours of infection. Such a method may also find use as part of a “block and lock” methodology, in which agents are used to prevent latently infected cells from reactivating. Active agents utilized in a block and lock methodology, include, e.g., the Tat inhibitor didehydro-Cortistatin A (Kessing, C F et al. Cell Rep. 2017 Oct. 17; 21(3):600-611. doi: 10.1016/j.celrep.2017.09.080.).

The present disclosure further provides a drug delivery device comprising: a) a first container comprising a FOXO1 inhibitor that reactivates latent immunodeficiency virus transcription; and b) a second container comprising an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity. The first and second containers can be syringes, vials, or ampules.

The present disclosure further provides a drug delivery device comprising: a) a first container comprising an endoplasmic reticulum (ER) stress-inducing agent, e.g., fenretinide or bortezomib, that reactivates latent immunodeficiency virus transcription; and b) a second container comprising an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity. The first and second containers can be syringes, vials, or ampules.

The present disclosure further provides a drug delivery device comprising: a) a first container comprising a FOXO1 inhibitor that reactivates latent immunodeficiency virus transcription; and b) a second container comprising an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof.

The present disclosure further provides a drug delivery device comprising: a) a first container comprising an endoplasmic reticulum (ER) stress-inducing agent, e.g., fenretinide or bortezomib, that reactivates latent immunodeficiency virus transcription; and b) a second container comprising an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof.

In some embodiments of a method of the present disclosure, or a device of the present disclosure, the FOXO1 inhibitor is a small molecule FOXO1 inhibitor, e.g., AS1842856 (5-amino-7-(cyclohexylamino)-1-ethyl-6-fluoro-4-oxo-1,4-dihydro-quinoline-3-carboxylic acid) or a pharmaceutically acceptable salt thereof. In some embodiments of a method of the present disclosure, or a device of the present disclosure, the FOXO1 inhibitor is an siNA targeting expression of FOXO1, or a nucleic acid encoding an siNA targeting expression of FOXO1. In some embodiments of a method of the present disclosure, or a device of the present disclosure, the FOXO1 inhibitor is an siNA comprising a FOXO1 shRNA nucleotide sequence. In some embodiments of a method of the present disclosure, or a device of the present disclosure, the FOXO1 inhibitor is a nucleic acid comprising a nucleotide sequence encoding an siNA comprising a FOXO1 shRNA nucleotide sequence. In some embodiments of a method of the present disclosure, or a device of the present disclosure, the FOXO1 inhibitor is an expression vector comprising a nucleotide sequence encoding an siNA comprising a FOXO1 shRNA nucleotide sequence.

### BRIEF DESCRIPTION OF THE DRAWINGS

FIG. 1, Panels A-E depict FOXO1 as a specific regulator of HIV latency establishment. FIG. 1, Panel A (left, top) is a schematic representation of HIV_{GKO} dual-labeled HIV-1 reporter. FIG. 1, Panel A (left, bottom) shows the amount of productive or latent cells relative to the total infection rate (bars) and cell viability (dots). FIG. 1, Panel A (right) shows ratios of productive versus latent populations upon increasing concentrations of AS1842856 treatments. FIG. 1, Panel B depicts FOXO1, FOXO3 and FOXO4 Cellular Thermal Shift Assay (CETSA)-melting curve shifts upon the presence or absence of AS1842856 1000 nM in K562 cells. FIG. 1, Panel C depicts the efficiency of FOXO1, FOXO3 and FOXO4 knockdowns with two different sgRNAs checked by western blot. FIG. 1, Panel D (left) depicts percentage of productive or latent cells relative to the total infection rate and cell viability in the different single knockdown K562 cell lines. FIG. 1, Panel D (right) depicts ratios of productive versus latent populations in the different K562 knockdown cell lines for the different FOXO proteins. FIG. 1, Panel E depicts ratios of productive versus latent populations upon increasing concentrations of AS1842856 treatments in the WT K562 or in the FOXO1-knockdown cell lines.

FIG. 2, Panels A-I depict the role of other FOXO family members in HIV-1 latency establishment. FIG. 2, Panel A shows representative western blots of CETSA assays in FOXO1 and FOXO3 in the presence or absence of 100 nM AS1842856. FIG. 2, Panel B shows FOXO1 and FOXO3 CETSA-melting curves upon the presence or absence of AS1842856 100 nM in K562 cells. FIG. 2, Panel C shows cell viability results of CETSA assays in FOXO1 experiments. FIG. 2, Panels D-F show percentage of productive or latent cells relative to the total infection rate and cell viability upon increasing concentrations of AS1842856 treatment in the WT K562 (FIG. 2, Panel D) or in the FoxO1-knockdown cell lines KD-72 (FIG. 2, Panel E) and KD-73 (FIG. 2, Panel F). FIG. 2, Panel G shows a cell growth analysis of WT, FOXO1, FOXO3 and FOXO4 single knockdowns K562 cell lines. FIG. 2, Panel H shows percentage of productive or latent cells relative to the total infection rate and cell viability in double knockdowns K562 cell lines. FIG. 2, Panel I shows ratios of productive versus latent populations upon increasing concentrations of AS1842856 treatments in double knockdowns K562 cell lines. Data are represented as mean±SD of two independent experiments.

FIG. 3, Panels A-E demonstrate that FOXO1 inhibition reactivates HIV1 from latency. FIG. 3, Panel A shows a J-Lat cell line 5A8 treated with increasing concentrations (1-10000 nM) of AS1842856 for 24, 48 and 72 h. HIV-GFP mRNA reactivation was assessed by RT-qPCR and normalized to RPL13AmRNA. FIG. 3, Panel B depicts cell viability assessed by flow cytometry at 72 h of the same experiment as in A. Cell viability was measured by gating on both the live population at the forward scatter (FSC) and side scatter (SSC) plot and then the viable cells after staining with the viability dye. FIG. 3, Panel C depicts analysis of J-Lat cell lines A2 and A72 that were treated with increasing concentrations of AS1842856 for 24, 48 and 72 h. HIV-GFP reactivation (bars) and cell viability (dots) were analyzed by FACS. HIV-GFP reactivation is reported as a percentage of GFP-expressing cells (% GFP+ cells). Within each of the concentrations of AS1842856, data groupings from left to right are J-Lat cell lines A2 and A72. FIG. 3, Panel D depicts the same experiments as in FIG. 3, Panel C but performed in different J-Lat cell lines 5A8, 6.3, 11.1 and 15.4. Within each of the concentrations of AS1842856, data groupings from left to right are J-Lat cell lines 5A8, 6.3, 11.1 and 15.4. 10 ng/mL. TNFα was used as control. FIG. 3, Panel E depicts results for J-Lat cell lines 5A8, 6.3, 11.1 and 15.4 that were treated for 72 h with increasing concentrations of both AS1842856 (Y-axis) and TNFα (X-axis) alone or in combination and analyzed by FACS.

FIG. 4, Panels A-D depict results of J-Lat cell lines treated with different concentrations of AS1842856. FIG. 4, Panel A depicts results for J-Lat cell lines A2 and A72 treated with increasing concentrations of AS1842856 for 24, 48 and 72 h. HIV-GFP reactivation was analyzed by flow cytometry. HIV-GFP reactivation is reported as a Mean Intensity Fluorescence (MFI) of GFP-expressing cells. Within each of the concentrations of AS1842856, data groupings from left to right are J-Lat cell lines A2 and A72. FIG. 4, Panel B depicts results for the same experiments as in FIG. 4, Panel A but performed in different J-Lat cell lines 5A8, 6.3, 11.1 and 15.4. Within each of the concentrations of AS1842856, data groupings from left to right are J-Lat cell lines 5A8, 6.3, 11.1 and 15.4. 10 ng/mL TNFα was used as control. FIG. 4, Panel C depicts results for J-Lat cell lines 5A8, 6.3, 11.1 and 15.4 that were treated for 72 h with increasing concentrations of both AS1842856 (Y-axis) and TNFα (X-axis) alone or in combination and analyzed by FACS. HIV-GFP reactivation is reported as a percentage of GFP-expressing cells (% GFP+ cells) or Mean Intensity Fluorescence (MFI) (FIG. 4, Panel D).

FIG. 5, Panels A-C demonstrate that FOXO1 inhibitions prevent latency establishment and reactivates HIV in primary CD4⁺ T cells and HIV-infected CD4⁺ T cells. FIG. 5, Panel A depicts a schematic representation of HIV_{GKO} dual-labeled HIV-1 reporter and strategy used to treat primaryCD4⁺ T cells purified from blood of healthy donors and pre-treated for 24 h with increasing concentrations of AS1842856. Ratios of productive versus latent populations of infected CD4⁺ T cells from four different healthy donors after treatment with increasing concentrations of AS1842856 are shown in FIG. 5, Panel A. A histogram plot of percent live cells for each drug treatment relative to the control are also shown. FIG. 5, Panel B depicts a schematic representation of HIV_{NL4-3 Luciferase} reporter virus and an experimental procedure with primary CD4+T cells. FIG. 5, Panel C depicts results for AS1842856/prostratin-treated CD4+ T cells in HIV-infected patients on antiretroviral-therapy with undetectable viral load.

FIG. 6, Panels A-D demonstrate that FoxO1 inhibition prevents latency establishment and reactivates HIV in primary CD4+ T cells. FIG. 6, Panel A depicts results for measurement of cell surface CD69 and CD25 T cell activation markers that were measured by FACS in CD4+ T cells upon AS1842856 treatment for 24, 48 and 72 h. 10 μg/mLαCD3 and 1 μg/mL αCD28 was used as control. FIG. 6, Panel B depicts the same experiment as in FIG. 6, Panel A but measuring FOXO1 target genes (IL7R, KLRG1, CD62L) after 48 h of AS1842856 treatment (n=3). For each of the target genes, data groupings from left to right are DMSO, 10 nM FOXO1i, 100 nM FOXO1i, and 1000 nM FOXO1i. F FIG. 6, Panel C, depicts HIV reactivation measured by luciferase activity and cell viability measured by flow cytometry assessed in CD4⁺ T cells purified from blood of healthy donors and infected with HIV_{NL4-3 Luciferase VSVg-pseudotyped}. Cells were allowed to rest for 6 days before 72 h reactivation was induced with PHA/IL-2 and 10 μg/mL αCD3 and 1 μg/mLαCD28. FIG. 6, Panel D depicts fold change of cell-associated HIV mRNA measured by ddPCR of CD4+ T cells of nine HIV-infected patients on antiretroviral-therapy with undetectable viral load treated with PMA and Ionomycin. Cell viability was assessed by Trypan Blue exclusion.

FIG. 7, Panels A-E depict marked upregulation of the regulator ATF4 in response to FOXO1 inhibition in primary CD4+ T cells. FIG. 7, Panel A depicts a volcano plot from the RNA-Seq data comparing CD4+ T cells treated with 1000 nM AS1842856 versus DMSO control for 48 h. Up-regulated or down-regulated genes are q-value<0.05 and log 2 fold change≥1 or ≤−1, respectively. FIG. 7, Panel B depicts analysis of the most dysregulated canonical pathways for the up- and down-regulated genes by Ingenuity Pathway Analysis. FIG. 7, Panel C depicts confirmation of up- or down-regulated expression of specific genes from different altered pathways by mean read counts from sequencing. FIG. 7, Panel D depicts analysis of the five top upstream regulators and table with their activation z-scores according to the RNA-Seq data. FIG. 7, Panel E depicts a representative blot of protein expression of dysregulated genes of interest (FIG. 7, Panel E, left) and densitometry analysis (FIG. 7, Panel E, right) was performed from three individual donors.

FIG. 8, Panels A-D depict read counts of the selected genes representative of different pathways as well as Real-Time Quantitative PCR (RT-qPCR) results. FIG. 8, Panel A depicts a volcano plot from the RNA-Seq data comparing CD4+ T cells treated with 1000 nM AS1842856 versus DMSO control for 12 h. Up-regulated or down-regulated genes are q-value<0.05 and log 2 fold change≥1 or ≤−1, respectively. FIG. 8, Panel B depicts analysis of the most dysregulated canonical pathways for the up- and down-regulated genes at 12 h by Ingenuity Pathway Analysis. FIG. 8, Panels C-D depict confirmation of up- or down-regulated expression of specific genes from different altered pathways by mean counts from sequencing at 12 h (FIG. 8, Panel C) and 48 h (FIG. 8, Panel D). For each of the genes, data groupings from left to right are control and AS1842856 (1000 nM).

FIG. 9, Panels A-G depict that FOXO1 Inhibition induces HIV reactivation in the absence of NF-kB recruitment via ATF4 and NFAT. FIG. 9, Panel A depicts results for chromatin immunoprecipitation (ChIP) assays with antibodies against Pol III, ATF4, RelA, NFAT and IgG control at the HIV LTR, followed by qPCR using primers specific for HIV-1 LTR Nuc0 or Nuc1. For each of the genes, data groupings from left to right are control and AS1842856 (FIG. 9, Panel A, middle—control, AS1842856, and TNFα). FIG. 9, Panel B depicts results for a J-Lat cell line A58 that was treated (and pre-treated for 1 h) with increasing concentrations of GSK2656157 (PERK inhibitor II) in combination with 1000 nM AS1842856 (72 h) or 10 ng/mL TNFα (24 h). HIV-GFP reactivation was analyzed by FACS and relativized to the control condition. FIG. 9, Panel C depicts results for the same experiment as in FIG. 9, Panel B but cells were treated with increasing concentrations of Cyclosporin A (CsA) and without 1 h pre-treatment. FIG. 9, Panel D depicts results for a similar experiment as that in FIG. 9, Panel B or Panel C, but using increasing concentrations of GSK2656157 (PERK inhibitor II) and Cyclosporin A (CsA) combined. FIG. 9, Panel E depicts results for a J-Lat cell line A58 that was treated with increasing concentrations of Thapsigargin (0.01, 0.1, 1 μM), Brefeldin A (0.01, 0.1, 1 μg/mL) and Fenretinide (0.5, 2, 5 μM) for 24, 48 and 72 h and HIV-GFP reactivation was analyzed by FACS. FIG. 9, Panel F depicts a J-Lat cell line A58 that was treated with 0.5 μM Fenretinide and increasing concentrations of Ionomycin. HIV-GFP reactivation and cell viability were analyzed by FACS. For each Ionomycin concentration, the left bar shows relative GFP expression and the right bar shows cell viability. FIG. 9, Panel G is a schematic representation of FOXO1 inhibition that leads to ER Stress.

FIG. 10, Panels A-F depict results of a J-Lat cell line 5A8 treated with different concentrations of inhibitors. FIG. 10, Panel A depicts results for a J-Lat cell line A58 that was treated with increasing concentrations of A-92 (GCN2 inhibitor) in combination with 1000 nM AS1842856 (72 h) or 10 ng/mL TNFα (24 h). Histogram plots of percent live cells for each drug treatment are shown. FIG. 10, Panel B depicts results for the same experiment as in FIG. 10, Panel A, but cells were treated with increasing concentrations of PKR inhibitor (PKRi). Histogram plots representing viability are also shown. FIG. 10, Panel C depicts histogram plots of percent live cells for each drug treatment. Increasing concentrations of GSK2656157 (PERK inhibitor II) (FIG. 10, Panel C, top left), Cyclosporin A (CsA) (FIG. 10, Panel C, top right), combined concentrations of GSK2656157 and Cyclosporin A (FIG. 10, Panel C, bottom left) and increasing concentrations of Thapsigargin (0.01, 0.1, 1 μM), Brefeldin A (0.01, 0.1, 1 μg/mL) and Fenretinide (0.5, 2, 5 μM) for 24, 48 and 72 h (FIG. 10, Panel C, bottom right). Within each of the −, +, ++, and +++ categories of FIG. 10, Panel C, bottom right, data groupings from left to right are Thapsigargin, Brefeldin A, and Fenretinide. FIG. 10, Panel D depicts results for a J-Lat cell line A58 that was treated with increasing concentrations of Ionomycin (0.01, 0.1, 0.5, 1 μM) for 24, 48 and 72 h. HIV-GFP reactivation and cell viability were analyzed by FACS. FIG. 10, Panel E depicts results for a J-Lat cell line 5A8 that was treated for 72 h with increasing concentrations of both Fenretinide (Y-axis) and Ionomycin (X-axis) alone or in combination and analyzed by FACS. HIV-GFP reactivation is reported as a percentage of GFP-expressing cells (% GFP+ cells). FIG. 10, Panel F depicts results for a J-Lat cell line 5A8 that was also treated for 72 h with increasing concentrations of both Thapsigargin (Y-axis) and Cyclosporin A (X-axis) alone or in combination and analyzed by FACS. HIV-GFP reactivation is again reported as a percentage of GFP-expressing cells (% GFP+ cells). p-value: *p<0.05, **p<0.01, ***p<0.001, ****p<0.0001.

FIG. 11 depicts the amino acid sequence of a human FOXO1 polypeptide.

### DEFINITIONS

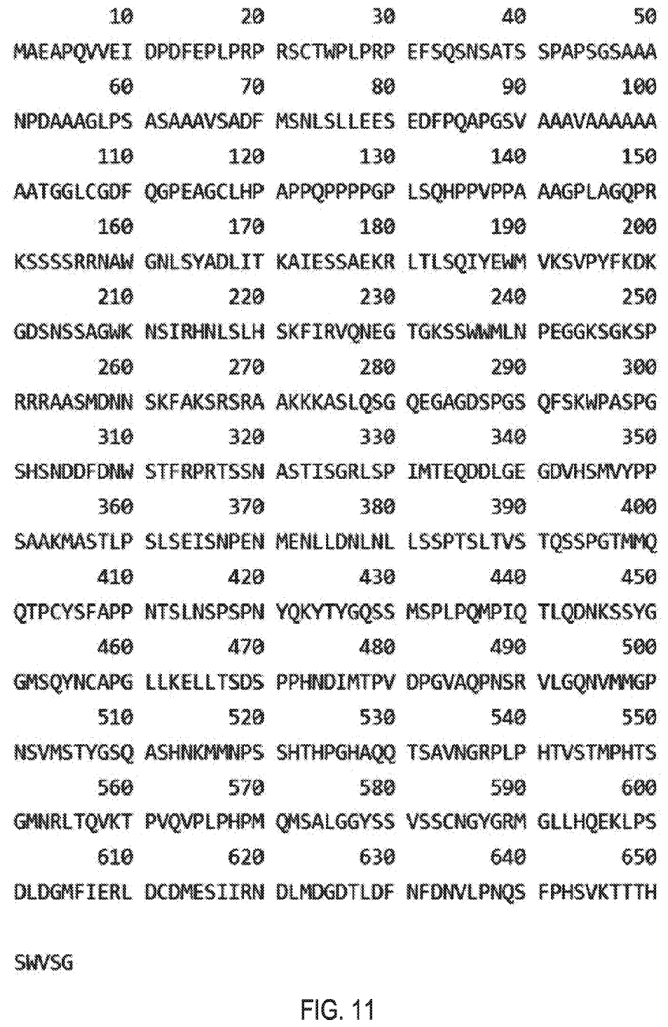

As used herein, “FOXO1” (also known as Forkhead Box O1, Forkhead Box Protein O1A, FOXO1A, FKHR; Forkhead, Drosophila, Homolog Of, In Rhabdomyosarcoma; Forkhead Homolog In Rhabdomyosarcoma; Forkhead In Rhabdomyosarcoma; Forkhead Box Protein O1; and FKH1) refers to a polypeptide comprising an amino acid sequence having at least about 80%, at least about 85%, at least about 90%, at least about 95%, at least about 98%, at least about 99%, or 100%, amino acid sequence identity to the amino acid sequence set forth in FIG. 11. Structural information relating to FOXO1 is found in, e.g., Carrette et al. (2009).

The term “immunodeficiency virus” includes human immunodeficiency virus (HIV), feline immunodeficiency virus, and simian immunodeficiency virus. The term “human immunodeficiency virus” as used herein, refers to human immunodeficiency virus-1 (HIV-1); human immunodeficiency virus-2 (HIV-2); and any of a variety of HIV subtypes and quasispecies.

As used herein, the term “exogenously supplied”, as used in the context of an exogenously supplied immunodeficiency virus immunogen, e.g., an HIV-1 envelope, a fragment thereof, or a peptide derived from an HIV-1 envelope, refers to an immunodeficiency virus immunogen that is supplied, provided, contacted to a cell, administered, and the like, other than as a result of an immunodeficiency virus infection, e.g., by the hand of man.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a compound that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “pharmaceutical composition” is meant to encompass a composition suitable for administration to a subject, such as a mammal, especially a human. In general, a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like. In some embodiments the composition is suitable for administration by a transdermal route, using a penetration enhancer other than dimethylsulfoxide (DMSO). In other embodiments, the pharmaceutical compositions are suitable for administration by a route other than transdermal administration. A pharmaceutical composition will in some embodiments include a subject compound and a pharmaceutically acceptable excipient. In some embodiments, a pharmaceutically acceptable excipient is other than DMSO.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

As used herein the term “pharmaceutically acceptable salt” refers to a salt of a compound, which possesses the desired pharmacological activity of the compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3 (4 hydroxybenzoyl) benzoic acid, cinnamic acid, mandelic acid, methanesulfonic acid, ethanesulfonic acid, 1,2 ethane disulfonic acid, 2 hydroxyethanesulfonic acid, benzenesulfonic acid, 4 chlorobenzenesulfonic acid, 2 naphthalenesulfonic acid, 4 toluenesulfonic acid, camphorsulfonic acid, 4 methylbicyclo[2.2.2] oct 2 ene 1 carboxylic acid, glucoheptonic acid, 3 phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. It is understood, that the present disclosure controls to the extent there is a conflict with a publication incorporated by reference herein.

It must be noted that as used herein and in the appended claims, the singular forms “a,” “an,” and “the” include plural referents unless the context clearly dictates otherwise. Thus, for example, reference to “a FOXO1 inhibitor” includes a plurality of such inhibitors and reference to “the agent” includes reference to one or more agents and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication. Further, the dates of publication provided may be different from the actual publication dates which may need to be independently confirmed.

### DETAILED DESCRIPTION

The present disclosure provides methods of reactivating latent HIV integrated into the genome of an HIV-infected cell. The methods generally involve contacting the cell with a FOXO1 inhibitor that reactivates latent HIV integrated into the genome of the cell. In some embodiments, the contacting takes place in the absence of an exogenously supplied immunodeficiency virus immunogen.

The Forkhead box O (FOXO) protein family comprises evolutionarily conserved transcription factors. FOXO-mediated gene regulation is determined in part by localization of the proteins; upon phosphorylation by upstream kinases, they exit the nucleus and remain inactive. The four human FOXO isoforms influence multiple pathways including cell cycle arrest, glucose metabolism, oxidative stress regulation, apoptosis, and the DNA damage response. In the immune system, FOXO proteins regulate a set of genes involved in maintaining quiescence and cell fate differentiation in CD4+ and CD8+ T cells. Furthermore, inhibition of FOXO1 in naïve and memory CD8+ T cells promotes a more effector-like and cytotoxic phenotype in the context of immune aging and chronic infection.

The present disclosure provides methods for reducing the number of cells containing a latent human immunodeficiency virus in an individual, the method comprising administering to the individual an effective amount of a FOXO1 inhibitor that reactivates latent HIV integrated into the genome of one or more cells in the individual. In some embodiments, the administering does not comprise administering an immunodeficiency virus immunogen to the individual.

The present disclosure provides methods of treating an immunodeficiency virus infection in an individual, the methods generally involving co-administering to the individual an agent that reactivates latent HIV and an anti-HIV agent. For example, the present disclosure provides a method of treating a human immunodeficiency virus (HIV) infection in an individual, the method comprising: administering to an individual an effective amount of a first active agent, wherein the first active agent is a FOXO1 inhibitor that reactivates latent HIV integrated into the genome of a cell in the individual; and administering to the individual an effective amount of a second active agent, wherein the second active agent inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity. In some embodiments, the administering to the individual of an effective amount of a first active agent does not comprise administering an immunodeficiency virus immunogen to the individual.

The present disclosure also provides a method of treating a human immunodeficiency virus (HIV) infection in an individual, the method comprising: administering to an individual an effective amount of a first active agent, wherein the first active agent is a FOXO1 inhibitor that reactivates latent HIV integrated into the genome of a cell in the individual; and administering to the individual an effective amount of an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof. In some embodiments, the administering to the individual of an effective amount of a first active agent does not comprise administering an immunodeficiency virus immunogen to the individual.

An agent that inhibits activity of a FOXO1 polypeptide and/or that reduces the level of a FOXO1 polypeptide in a cell, and that activates latent HIV is referred to herein as a “FOXO1 inhibitor.” In some cases, a FOXO1 inhibitor suitable for use in a method of the present disclosure inhibits a transcription regulatory activity of FOXO1, e.g., a DNA-binding activity of FOXO1. In some cases, a FOXO1 inhibitor suitable for use in a method of the present disclosure reduces the level of a FOXO1 polypeptide in a cell. Regardless of the mechanism, a FOXO1 inhibitor suitable for use in a method of the present disclosure activates latent HIV in a cell harboring latent HIV.

In some cases, a suitable active agent for use in a method of the present disclosure for activating latent HIV is an agent that inhibits FOXO1activity by at least 10%, at least 20%, at least 30%, at least 40%, at least 50%, at least 60%, at least 70%, at least 80%, or at least 90%, compared to the activity of the FOXO1 polypeptide in the absence of the active agent. FOXO1 activity can be measured, e.g., by using assays as described herein.

In some cases, a suitable active agent for use in a method of the present disclosure for activating latent HIV is an agent that reduces the level of FOXO1 polypeptide in a cell by at least 10%, at least 20%, at least 30%, at least 40%, at least 50%, at least 60%, at least 70%, at least 80%, or at least 90%, compared to the level of the FOXO1 polypeptide in the cell in the absence of the agent.

An effective amount of an active agent that inhibits activity of a FOXO1 polypeptide and/or reduces the level of a FOXO1 polypeptide in a cell is an amount that reactivates latent HIV and reduces the reservoir of latent HIV in an individual, e.g., by at least about 10%, at least about 20%, at least about 30%, at least about 40%, at least about 50%, at least about 60%, at least about 70%, at least about 80%, or at least about 90%. A “reduction in the reservoir of latent HIV” (also referred to as “reservoir of latently infected cells”) is a reduction in the number of cells in the individual that harbor a latent HIV infection. Whether the reservoir of latently infected cells is reduced can be determined using any known method, including the method described in Blankson et al. (2000) J. Infect. Disease 182(6):1636-1642.

In some cases, an effective amount of a FOXO1 inhibitor is an amount that is effective to reduce the number of cells, in a cell population, present in an individual and containing a latent human immunodeficiency virus, by at least 10%, at least 20%, at least 30%, at least 40%, at least 50%, at least 60%, at least 70%, at least 80%, or at least 90%. The cell population can be a population of HIV-infected cells in an individual.

### Active Agents

Suitable active agents include agents that inhibit the activity of a FOXO1 polypeptide and/or reduce the level of a FOXO1 polypeptide in a cell. In some embodiments, suitable active agents include agents that increase the activity of a FOXO1 polypeptide and/or increase the level of a FOXO1 polypeptide in a cell. Suitable active agents include FOXO1 inhibitors that reactivate latent immunodeficiency virus (e.g., HIV) in a cell. In some embodiments, suitable active agents include FOXO1 activators that suppress latency establishment and/or maintenance in a cell. Suitable active agents include ER stress-inducing agents that reactivate latent immunodeficiency virus (e.g., HIV) in a cell. In some embodiments, suitable active agents include ER stress-inhibiting agents that suppress latency establishment and maintenance in a cell.

### Small Molecule Inhibitors or Activators

In some cases, the active agent is a cell-permeable inhibitor or activator of a transcription regulatory activity, e.g., DNA binding activity, of a FOXO1 polypeptide. In some cases, the active agent is a cell-permeable inhibitor or activator of a transcription regulatory activity a FOXO1 polypeptide, where the active agent is a selective FOXO1 inhibitor or activator. In some embodiments, a selective FOXO1 inhibitor or activator does not substantially inhibit or activate, respectively, a FOXO3 polypeptide, a FOXO4 polypeptide, or a FOXO6 polypeptide.

In some cases, the active agent is a cell-permeable inhibitor of a transcription regulatory activity of a FOXO1 polypeptide; and the active agent has an IC₅₀ of from about 0.001 μM to about 100 μM. In some cases, the active agent is a cell-permeable inhibitor of a transcription regulatory activity of a FOXO1 polypeptide; and the active agent has an IC₅₀ of from about 0.001 μM to about 10 μM. In some cases, the active agent is a cell-permeable inhibitor of a transcription regulatory activity of a FOXO1 polypeptide; and the active agent has an IC₅₀ of from about 0.001 μM to about 1 μM. In some cases, the active agent is a cell-permeable inhibitor of a transcription regulatory activity of a FOXO1 polypeptide; and the active agent has an IC₅₀ of from about 0.001 μM to about 0.002 μM, from about 0.002 μM to about 0.003 μM, from about 0.003 μM to about 0.005 μM, from 0.005 μM to about 0.010 μM, from about 0.010 μM to about 0.015 μM, from about 0.015 μM to about 0.02 μM, from about 0.02 μM to about 0.05 μM, from about 0.05 μM to about 0.1 μM, from about 0.1 μM to about 0.5 μM, or from about 0.5 μM to about 1.0 μM. In some cases, the active agent is a cell-permeable inhibitor of a transcription regulatory activity of a FOXO1 polypeptide; and the active agent has an IC₅₀ of from about 1.0 μM to about 5 μM, from about 5 μM to about 10 μM, from about 10 μM to about 25 μM, from about 25 μM to about 50 μM, from about 50 μM to about 75 μM, or from about 75 μM to about 100 μM. In some cases, the active agent is a cell-permeable inhibitor of a transcription regulatory activity of a FOXO1 polypeptide; and the active agent has an IC₅₀ of from about 100 μM to about 1 nM. In some cases, the active agent is a cell-permeable inhibitor of a transcription regulatory activity of a FOXO1 polypeptide; and the active agent has an IC₅₀ of from about 1 nM to about 50 nM. In some cases, the active agent is a cell-permeable inhibitor of a transcription regulatory activity of a FOXO1 polypeptide; and the active agent has an IC₅₀ of from about 50 nM to about 100 nM. In some cases, the active agent has an IC₅₀ of about 30 nM.

An example of a suitable active agent is AS1842856 or a pharmaceutically acceptable salt thereof. AS1842856 (5-amino-7-(cyclohexylamino)-1-ethyl-6-fluoro-4-oxo-1,4-dihydro-quinoline-3-carboxylic acid) is a selective FOXO1 inhibitor. Trinité et al. (2014). AS1842856 has the following structure:

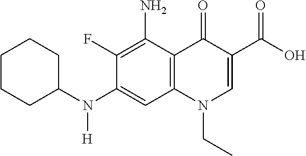 [SMILES: CCn1cc(C(=O)O)c(=O)c2c(N)c(F)c(NC3CCCCC3)cc21]

In some embodiments, AS1842856 is administered or formulated as a pharmaceutically acceptable salt, including but not limited to salts formed when an acidic proton present in the compound is replaced by a metal ion, e.g., an alkali metal ion, an alkaline earth ion, or an aluminum ion; or coordinates with an organic base. In certain embodiments, pharmaceutically acceptable salts of AS1842856 include cationic counterion salts having a cationic counterion such as Aluminum, Arginine, Benzathine, Calcium, Chloroprocaine, Choline, Diethanolamine, Ethanolamine, Ethylenediamine, Lysine, Magnesium, Histidine, Lithium, Meglumine, Potassium, Procaine, Sodium, Triethylamine and Zinc among others.

Additional small molecule FOXO1 inhibitors, which may find use in the methods and compositions of the present disclosure include, but are not limited to:

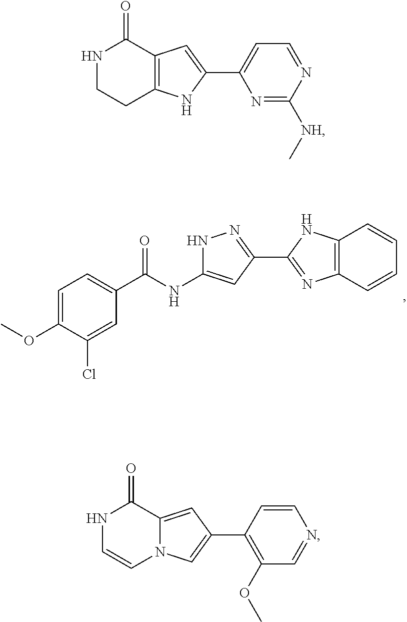 [SMILES: CNc1nccc(-c2cc3c([nH]2)CCNC3=O)n1 ; COc1ccc(C(=O)Nc2cc(-c3nc4ccccc4[nH]3)n[nH]2)cc1Cl ; COc1cnccc1-c1cc2c(=O)[nH]ccn2c1]

and pharmaceutically acceptable salts thereof.

Combinations of two or more FOXO1 inhibitors or activators can also be used in a method of the present disclosure as appropriate. Furthermore, in a method including reactivating latent HIV, any one or more of the FOXO1 inhibitors as described herein may be used in combination with any one or more of the endoplasmic reticulum (ER) stress-inducing agents described herein (with or without a Ca²⁺ mobilizing agent) or any one or more of the additional latency reversal agents described herein.

Other examples of suitable agents, e.g., ER stress-inducing agents, include fenretinide, bortezomib, theaflavin-3,3′-digallate (TF-3), inositol-requiring protein-1 (IRE1), 3,4-dimethoxycinnamic acid, 3β-hydroxyisoallospirost-9(11)-ene, actinomycin, aminopterin, calcium gluceptate, cephradine, chloramphenicol, chlorothiazide, chloroxylenol, cisplatin, curcumin, cysteamine, deracoxib, epiafzelechin, euparin, flurbiprofen, gabexate mesylate, gedunin, gemfibrozil, halcinonide, ibuprofen, liothyronine, lobendazole, metoclopramide, naltrexone, niacinamide, pregnenolone, pyrazinamide, pyrromycin, ramipril, succinylsulfathiazole, sulindac, and theaflavin digallate. Such suitable agents may be used in conjunction with or without Ca²⁺ mobilizers, e.g., ionomycin.

Other examples of suitable agents include FOXO1 activating agents and ER stress-inhibiting agents as appropriate.

### Nucleic Acids Encoding FOXO1

Examples of FOXO1 activating agents include nucleic acids encoding FOXO1, e.g., nucleic acids encoding a polypeptide having the sequence set forth in FIG. 11, which nucleic acids may be introduced into a suitable cell, e.g., using a suitable delivery vector or any other suitable method known in the art. The nucleic acid can be present in a recombinant expression vector, e.g., a recombinant viral vector (e.g., a lentivirus-based vector; an adeno-associated virus-based vector; and the like). Suitable promoters include those that are functional in a mammalian cell, e.g., a CD4⁺ T cell. A suitable promoter includes, e.g., a CD4 promoter.

### Nucleic Acid Inhibitors

In some cases, an active agent is a short interfering nucleic acid (siNA). The terms “short interfering nucleic acid,” “siNA,” “short interfering RNA,” “siRNA,” “shRNA,” “short interfering nucleic acid molecule,” “short interfering oligonucleotide molecule,” and “chemically-modified short interfering nucleic acid molecule” as used herein refer to any nucleic acid molecule capable of inhibiting or down regulating gene expression, for example by mediating RNA interference “RNAi” or gene silencing in a sequence-specific manner. As used herein, siNA includes short hairpin RNA (shRNA), short interfering RNA (siRNA), and the like.

A nucleic acid encoding an siNA is also contemplated for use in a method of the present disclosure, where the nucleic acid comprises a nucleotide sequence encoding the siNA. A nucleic acid encoding an siNA that reduces the level of FOXO1 polypeptide in a cell can comprise a promoter operably linked to the nucleotide sequence encoding the siNA. The nucleic acid can be present in a recombinant expression vector, e.g., a recombinant viral vector (e.g., a lentivirus-based vector; an adeno-associated virus-based vector; and the like). Suitable promoters include those that are functional in a mammalian cell, e.g., a CD4⁺ T cell. A suitable promoter includes, e.g., a CD4 promoter.

In some embodiments, siNA is produced by methods not requiring the production of dsRNA, e.g., chemical synthesis or de novo synthesis or direct synthesis. Chemically synthesized siRNA may be synthesized on a custom basis or may be synthesized on a non-custom or stock or pre-designed basis. Custom designed siRNA are routinely available from various manufactures (e.g., Ambion®, a division of Life Technologies®, Grand Island, N.Y.; Thermo Scientific®, a division of Fisher Scientific®, Pittsburgh, Pa.; Sigma-Aldrich®, St. Louis, Mo.; Qiagen®, Hilden, Germany; etc.) which provide access to various tools for the design of siRNA. Tools for the design of siNA allow for the selection of one or more siRNA nucleotide sequences based on computational programs that apply algorithms on longer input nucleotide sequences to identify candidate siNA sequences likely to be effective in producing an RNAi effect. Such algorithms can be fully automated or semi-automated, e.g., allowing for user input to guide sRNA selection. Programs applying algorithms for siNA sequence selection are available remotely on the World Wide Web, e.g., at the websites of manufacturers of chemically synthesized siNA or at the websites of independent, e.g. open source, developers or at the websites of academic institutions. Programs applying algorithms for siRNA sequence selection may also be obtained, e.g., downloaded or received on compact disk as software. Such programs are well known in the art, see e.g., Naito et al. (2004) Nucleic Acids Research, 32:W124-W129; Boudreau et al. (2013) Nucleic Acids Research, 41:e9; Mysara et al. (2011) PLoS, 6:e25642; and Iyer et al. (2007) Comput Methods Programs Biomed, 85:203-9, which are incorporated herein by reference.

Publicly available tools to facilitate design of siNAs are available in the art. See, e.g., DEQOR: Design and Quality Control of RNAi (available on the internet at http://deqor(dot)mpi-cbg(dot)de/deqor_new/input(dot)html). See also, Henschel et al. Nucleic Acids Res. 2004 Jul. 1; 32(Web Server issue):W113-20. DEQOR is a web-based program which uses a scoring system based on state-of-the-art parameters for siNA design to evaluate the inhibitory potency of siNAs. DEQOR, therefore, can help to predict (i) regions in a gene that show high silencing capacity based on the base pair composition and (ii) siNAs with high silencing potential for chemical synthesis. In addition, each siNA arising from the input query is evaluated for possible cross-silencing activities by performing BLAST searches against the transcriptome or genome of a selected organism. DEQOR can therefore predict the probability that an mRNA fragment will cross-react with other genes in the cell and helps researchers to design experiments to test the specificity of siRNAs or chemically designed siRNAs.

Design of RNAi molecules, when given a target gene, is routine in the art. See also US 2005/0282188 (which is incorporated herein by reference) as well as references cited therein. See, e.g., Pushparaj et al. Clin Exp Pharmacol Physiol. 2006 May-June; 33(5-6):504-10; Lutzelberger et al. Handb Exp Pharmacol. 2006; (173):243-59; Aronin et al. Gene Ther. 2006 March; 13(6):509-16; Xie et al. Drug Discov Today. 2006 January; 11(1-2):67-73; Grunweller et al. Curr Med Chem. 2005; 12(26):3143-61; and Pekaraik et al. Brain Res Bull. 2005 Dec. 15; 68(1-2):115-20. Epub 2005 Sep. 9.

The genomic and mRNA sequences of various human FOXO1 isoforms are publicly available, e.g., via the NCBI database. See, e.g., NCBI reference numbers NC_000013.11 Reference GRCh38.p12 Primary Assembly, XM_011535008.2, and XM_011535010.2.

Methods for design and production of siNAs to a desired target are known in the art, and their application to FOXO1 for the purposes disclosed herein will be readily apparent to the ordinarily skilled artisan, as are methods of production of siNAs having modifications (e.g., chemical modifications) to provide for, e.g., enhanced stability, bioavailability, and other properties to enhance use as therapeutics. In addition, methods for formulation and delivery of siNAs (e.g., siRNAs; shRNAs) to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which are incorporated herein by reference.

siNA molecules can be of any of a variety of forms. For example, the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. siNA can also be assembled from two separate oligonucleotides, where one strand is the sense strand and the other is the antisense strand, wherein the antisense and sense strands are self-complementary. In this embodiment, each strand comprises nucleotide sequence that is complementary to nucleotide sequence in the other strand; such as where the antisense strand and sense strand form a duplex or double stranded structure, for example wherein the double stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the siNA molecule are complementary to the target nucleic acid or a portion thereof).

Alternatively, the siNA can be assembled from a single oligonucleotide, where the self-complementary sense and antisense regions of the siNA are linked by a nucleic acid-based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof.

The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (e.g., where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell, 110, 563-574 and Schwarz et al., 2002, Molecular Cell, 10, 537-568), or 5′,3′-diphosphate.

In certain embodiments, the siNA molecule contains separate sense and antisense sequences or regions, wherein the sense and antisense regions are covalently linked by nucleotide or non-nucleotide linker molecules as is known in the art, or are alternately non-covalently linked by ionic interactions, hydrogen bonding, van der Waals interactions, hydrophobic interactions, and/or stacking interactions. In certain embodiments, the siNA molecules comprise a nucleotide sequence that is complementary to a nucleotide sequence of a target gene. In another embodiment, the siNA molecule interacts with nucleotide sequence of a target gene in a manner that causes inhibition of expression of the target gene.

As used herein, siNA molecules need not be limited to those molecules containing only RNA, but further encompass chemically-modified nucleotides and non-nucleotides. In certain embodiments, the short interfering nucleic acid molecules of the invention lack 2′-hydroxy (2′-OH) containing nucleotides. siNAs do not necessarily require the presence of nucleotides having a 2′-hydroxy group for mediating RNAi and as such, siNA molecules suitable for use in a method of the present disclosure optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.”

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In some embodiments, an siNA is an siRNA. In some embodiments, an siNA is a shRNA. In some embodiments, a DNA comprising a nucleotide sequence encoding an siRNA is used. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence a target gene (e.g., FOXO1) at the post-transcriptional level or the pre-transcriptional level. In a non-limiting example, epigenetic regulation of gene expression by siNA molecules suitable for use in a method of the present disclosure can result from siNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

siNA (e.g., siRNA; shRNA; etc.) molecules contemplated herein can comprise a duplex forming oligonucleotide (DFO) see, e.g., WO 05/019453; and US 2005/0233329, which are incorporated herein by reference). siNA molecules also contemplated herein include multifunctional siNA, (see, e.g., WO 05/019453 and US 2004/0249178).

siNA (e.g., siRNA, shRNA, etc.) molecules contemplated herein can comprise an asymmetric hairpin or asymmetric duplex. By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a loop region comprising about 4 to about 12 (e.g., about 4, 5, 6, 7, 8, 9, 10, 11, or 12) nucleotides, and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant an siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region.

Stability and/or half-life of siRNAs can be improved through chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein, describing various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; each of which is hereby incorporated in their totality by reference herein). In view of such teachings, similar modifications can be used as described herein to modify the siNA nucleic acid molecules of disclosed herein so long as the ability of siNA to promote RNAi is cells is not significantly inhibited.

Short interfering nucleic acid (siNA) molecules (e.g., siRNA, shRNA, etc.) having chemical modifications that maintain or enhance activity are contemplated herein. Such a nucleic acid is also generally more resistant to nucleases than an unmodified nucleic acid. Accordingly, the in vitro and/or in vivo activity should not be significantly lowered. Nucleic acid molecules delivered exogenously are generally selected to be stable within cells at least for a period sufficient for transcription and/or translation of the target RNA to occur and to provide for modulation of production of the encoded mRNA and/or polypeptide so as to facilitate reduction of the level of the target gene product.

Production of RNA and DNA molecules can be accomplished synthetically and can provide for introduction of nucleotide modifications to provide for enhanced nuclease stability. (see, e.g., Wincott et al., 1995, Nucleic Acids Res. 23, 2677; Caruthers et al., 1992, Methods in Enzymology 211, 3-19, incorporated by reference herein. In one embodiment, nucleic acid molecules of the invention include one or more (e.g., about 1, 2, 3, 4, 5, 6, 7, 8, 9, 10, or more) G-clamp nucleotides, which are modified cytosine analogs which confer the ability to hydrogen bond both Watson-Crick and Hoogsteen faces of a complementary guanine within a duplex, and can provide for enhanced affinity and specificity to nucleic acid targets (see, e.g., Lin et al. 1998, J. Am. Chem. Soc., 120, 8531-8532). In another example, nucleic acid molecules can include one or more (e.g., about 1, 2, 3, 4, 5, 6, 7, 8, 9, 10, or more) LNA “locked nucleic acid” nucleotides such as a 2′,4′-C methylene bicyclo nucleotide (see, e.g., Wengel et al., WO 00/66604 and WO 99/14226).

siNA molecules can be provided as conjugates and/or complexes, e.g., to facilitate delivery of siNA molecules into a cell. Exemplary conjugates and/or complexes includes those composed of an siNA and a small molecule, lipid, cholesterol, phospholipid, nucleoside, antibody, toxin, negatively charged polymer (e.g., protein, peptide, hormone, carbohydrate, polyethylene glycol, or polyamine). In general, the transporters described are designed to be used either individually or as part of a multi-component system, with or without degradable linkers. These compounds can improve delivery and/or localization of nucleic acid molecules into cells in the presence or absence of serum (see, e.g., U.S. Pat. No. 5,854,038). Conjugates of the siNA molecules described herein can be attached to biologically active molecules via linkers that are biodegradable, such as biodegradable nucleic acid linker molecules.

Reducing Expression and/or Function of a FOXO1 Gene

In some cases, a method of the present disclosure includes introducing into a target cell an agent that reduces or inhibits function of a gene product (e.g., an mRNA; a polypeptide) encoded by the FOXO1 gene. In some cases, an agent that reduces or inhibits function of a polypeptide gene product encoded by the FOXO1 gene is a small molecule inhibitor. In some cases, an agent that reduces or inhibits function of a polypeptide gene product encoded by the FOXO1 gene is an antibody that is specific for the polypeptide gene product and that inhibits function of the polypeptide gene product.

Reducing expression of a FOXO1 gene includes reducing the level of an mRNA encoded by the FOXO1 gene. Reducing expression of a FOXO1 gene includes reducing the level of a polypeptide encoded by the FOXO1 gene. Reduction in the level of a polypeptide encoded by a FOXO1 gene can be accomplished by introducing a loss-of-function mutation into the genome such that the level of functional protein is reduced. Reduction in the level of a polypeptide encoded by a FOXO1 gene can be accomplished by introducing a mutation that results in an inactive polypeptide encoded by the FOXO1 gene. Reduction in the level of a polypeptide encoded by a FOXO1 gene can be accomplished by introducing a mutation that results in reduced production of a polypeptide encoded by the FOXO1 gene; e.g., a mutation can be introduced that results in a frameshift, a deletion of all or part of the polypeptide, etc. Reducing the level of expression of a FOXO1 gene, or reducing the level of a gene product encoded by the FOXO1 gene, can be achieved using a microRNA.

Reducing the level of a polypeptide encoded by a FOXO1 gene can be achieved by reducing translation of an mRNA encoding the polypeptide, e.g., using an inhibitory RNA (RNAi) agent such as a short hairpin RNA (shRNA), a short inhibitory RNA (siRNA), and the like. RNAi that targets a FOXO1 gene can be produced using well-established methods. Reducing the level of a polypeptide encoded by a FOXO1 gene can be achieved by reducing the mRNA encoding the polypeptide post-transcriptionally, e.g., using an RNAi agent such as shRNA or siRNA that targets the mRNA encoded by the FOXO1 gene. The level of mRNA encoding a polypeptide encoded by a FOXO1 gene can be reduced by blocking transcription (e.g., using gene editing tools to either alter a promoter and/or enhancer sequence or to modulate transcription, or by using modified gene editing tools, e.g., CRISPRi, that can modify transcription without cutting the target DNA).

Agents that inhibit expression and/or function of a FOXO1 gene include, but are not limited to, a) an RNAi agent; b) a genome-editing agent; and c) a small molecule that directly inhibits a function of a polypeptide encoded by the FOXO1 gene. Suitable RNAi agents include, e.g., an shRNA or an siRNA that specifically targets an mRNA encoded by a FOXO1 gene. Suitable genome-editing agents (also referred to herein as “site-specific endonucleases”) include, e.g., Zinc finger nuclease; a TALEN; a CRISPR/Cas genome editing agent such as Cas9, Cpf1, CasX, and CasY; and the like.

### Nucleic Acid Modifications

In some embodiments, a FOXO1 inhibitor (e.g., a dsRNA, a siNA, etc.) has one or more modifications, e.g., a base modification, a backbone modification, etc., to provide the nucleic acid with an enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound; however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′-O-methyl modified nucleotides, 2′ Fluoro modified nucleotides, locked nucleic acid (LNA) modified nucleotides, peptide nucleic acid (PNA) modified nucleotides, nucleotides with phosphorothioate (PS) linkages, and a 5′ cap (e.g., a 7-methylguanylate cap (m7G)). Additional details and additional modifications are described below.

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases the melting temperature (Tm) of RNA:RNA duplexes but results in only small changes in RNA:DNA stability. It is stabile with respect to attack by single-stranded ribonucleases and is typically 5 to 10-fold less susceptible to DNases than DNA. It is commonly used in antisense oligos as a means to increase stability and binding affinity to the target message.

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siNAs to improve stability in serum or other biological fluids.

Locked nucleic acid (LNA) bases have a modification to the ribose backbone that locks the base in the C3′-endo position, which favors RNA A-type helix duplex geometry. This modification significantly increases Tm and is also very nuclease resistant. Multiple LNA insertions can be placed in an oligonucleotide (“oligo”) at any position except the 3′-end. Due to the large increase in Tm conferred by LNAs, they also can cause an increase in primer dimer formation as well as self-hairpin formation. In some cases, the number of LNAs incorporated into a single oligo is 10 bases or less.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some embodiments, a subject siNA (e.g., siNA, shRNA, etc.) has one or more nucleotides that are 2′-O-Methyl modified nucleotides. In some embodiments, a subject siNA (e.g., a dsRNA, a siNA, an shRNA, etc.) has one or more 2′ Fluoro modified nucleotides. In some embodiments, a subject nucleic acid (e.g., a dsRNA, a siNA, an shRNA, etc.) has one or more LNA bases. In some embodiments, a subject nucleic acid (e.g., a dsRNA, a siNA, an shRNA, etc.) has one or more nucleotides that are linked by a phosphorothioate bond (i.e., the subject nucleic acid has one or more phosphorothioate linkages). In some embodiments, a subject nucleic acid (e.g., a dsRNA, a siNA, an shRNA, etc.) has a 5′ cap (e.g., a 7-methylguanylate cap (m7G)). In some embodiments, a subject nucleic acid (e.g., a dsRNA, a siNA, an shRNA, etc.) has a combination of modified nucleotides. For example, a subject nucleic acid (e.g., a dsRNA, a siNA, etc.) can have a 5′ cap (e.g., a 7-methylguanylate cap (m7G)) in addition to having one or more nucleotides with other modifications (e.g., a 2′-O-Methyl nucleotide and/or a 2′ Fluoro modified nucleotide and/or a LNA base and/or a phosphorothioate linkage).

### Modified Backbones and Modified Internucleoside Linkages

Examples of suitable nucleic acids containing modifications include nucleic acids containing modified backbones or non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be basic (the nucleobase is missing or has a hydroxyl group in place thereof). Various salts (such as, for example, potassium or sodium), mixed salts and free acid forms are also included.

In some embodiments, a subject siNA comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in U.S. Pat. No. 5,602,240.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A subject siNA can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general, the incorporation of CeNA monomers into a DNA chain increases the stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630). LNAs and preparation thereof are also described in WO 98/39352 and WO 99/14226, as well as U.S. Patent Publication Nos. 20120165514, 20100216983, 20090041809, 20060117410, 20040014959, 20020094555, and 20020086998.

### Modified Sugar Moieties

A subject siNA can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A subject siNA may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

### Conjugates

Another possible modification of a subject siNA involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject siNA.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the 3′ terminus of an exogenous polynucleotide (e.g., an siNA). In some embodiments, a PTD is covalently linked to the 5′ terminus of an exogenous polynucleotide (e.g., an siNA). Exemplary PTDs include but are not limited to a minimal undecapeptide protein transduction domain (corresponding to residues 47-57 of HIV-1 TAT comprising YGRKKRRQRRR (SEQ ID NO:11)); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:12); Transportan GWTLNSAGYLLGKINLKALAALAKKIL (SEQ ID NO:13); KALAWEAKLAKALAKALAKHLAKALAKALKCEA (SEQ ID NO:14); and RQIKIWFQNRRMKWKK (SEQ ID NO:15). Exemplary PTDs include but are not limited to, YGRKKRRQRRR (SEQ ID NO:11), RKKRRQRRR (SEQ ID NO:16); an arginine homopolymer of from 3 arginine residues to 50 arginine residues; Exemplary PTD domain amino acid sequences include, but are not limited to, any of the following: YGRKKRRQRRR (SEQ ID NO:11); RKKRRQRR (SEQ ID NO:17); YARAAARQARA (SEQ ID NO:18); THRLPRRRRRR (SEQ ID NO:19); and GGRRARRRRRR (SEQ ID NO:20). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Methods Utilizing Combinations of Active Agents and Combination Therapies

The present disclosure provides methods utilizing combinations of active agents and combination therapies for treating an immunodeficiency virus infection in an individual.

Combination Therapy—Two or More Agents that Reactivate Latent HIV

In some embodiments, the present disclosure provides a method of reactivating latent human immunodeficiency virus (HIV) integrated into the genome of a cell infected with HIV, the method including contacting the cell with a FOXO1 inhibitor that reactivates latent HIV integrated into the genome of the cell. In some embodiments, the method includes contacting the cell with at least one second agent that reactivates latent HIV. In some embodiments, alternatively or additional, the method includes contacting the cell with an effective amount of antiretroviral drug.

In some embodiments, a method of the present disclosure of treating an immunodeficiency virus infection in an individual in need thereof involves administering to the individual an effective amount of two or more agents that activate immunodeficiency virus transcription. In some cases, the two or more agents act synergistically to reactivate latent immunodeficiency virus.

In some cases, a method of the present disclosure of treating an immunodeficiency virus infection in an individual in need thereof involves: a) administering to the individual an effective amount of a FOXO1 inhibitor that activates immunodeficiency virus transcription; and b) administering to the individual an effective amount of a second agent that activates latent immunodeficiency virus transcription.

Suitable second agents that activate latent immunodeficiency virus transcription include, e.g., a bromodomain inhibitor; a protein kinase C (PKC) activator, such as prostratin, bryostatin, a chemical analog of prostratin, a chemical analog of bryostatin, and the like; a histone deacetylase (HDAC) inhibitor such as suberoylanilidehydroxamic (SAHA), romidepsin, sodium butyrate; a Symyd2 inhibitor, and the like.

Bromodomain inhibitors suitable for use include, e.g., JQ1, which has the following structure:

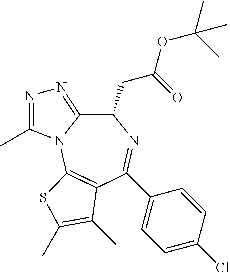 [SMILES: Cc1sc2c(c1C)C(c1ccc(Cl)cc1)=N[C@@H](CC(=O)OC(C)(C)C)c1nnc(C)n1-2]

Suitable bromodomain inhibitors include compounds of formula I:

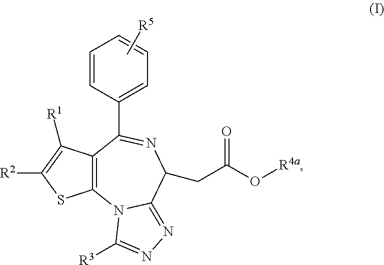

wherein

R1 is selected from hydrogen, alkyl, substituted alkyl, alkenyl, substituted alkenyl, alkynyl, substituted alkynyl, alkoxy, substituted alkoxy, and acyl;

R2 is selected from hydrogen, alkyl, substituted alkyl, alkenyl, substituted alkenyl, alkynyl, substituted alkynyl, alkoxy, substituted alkoxy, and acyl;

R3 is selected from hydrogen, alkyl, substituted alkyl, alkenyl, substituted alkenyl, alkynyl, substituted alkynyl, alkoxy, substituted alkoxy, and acyl;

R4a is selected from hydrogen, C1-C3 alkyl, C5-C10 alkyl, and substituted alkyl;

R5 is selected from hydrogen, alkyl, substituted alkyl, alkenyl, substituted alkenyl, alkynyl, substituted alkynyl, hydroxy, alkoxy, substituted alkoxy, acyloxy, thiol, acyl, amino, substituted amino, aminoacyl, acylamino, azido, carboxyl, carboxylalkyl, cyano, halogen, and nitro;

and salts or solvates or stereoisomers thereof.

In formula I, R¹ is selected from hydrogen, alkyl, substituted alkyl, alkenyl, substituted alkenyl, alkynyl, substituted alkynyl, alkoxy, substituted alkoxy, and acyl. In certain instances, R¹ is hydrogen. In certain instances, R¹ is alkyl or substituted alkyl. In certain instances, R¹ is alkyl, such as C₁-C₆ alkyl, including C₁-C₃ alkyl. In certain instances, R¹ is methyl, ethyl, n-propyl, or isopropyl. In certain instances, R¹ is methyl. In certain instances, R¹ is alkenyl or substituted alkenyl. In certain instances, R¹ is selected from alkynyl or substituted alkynyl. In certain instances, R¹ is alkoxy or substituted alkoxy. In certain instances, R¹ is acyl.

In formula I, R² is selected from hydrogen, alkyl, substituted alkyl, alkenyl, substituted alkenyl, alkynyl, substituted alkynyl, alkoxy, substituted alkoxy, and acyl. In certain instances, R² is hydrogen. In certain instances, R² is alkyl or substituted alkyl. In certain instances, R² is alkyl, such as C₁-C₆ alkyl, including C₁-C₃ alkyl. In certain instances, R² is methyl, ethyl, n-propyl, or isopropyl. In certain instances, R² is methyl. In certain instances, R² is alkenyl or substituted alkenyl. In certain instances, R² is selected from alkynyl or substituted alkynyl. In certain instances, R² is alkoxy or substituted alkoxy. In certain instances, R² is acyl.

In formula I, R³ is selected from hydrogen, alkyl, substituted alkyl, alkenyl, substituted alkenyl, alkynyl, substituted alkynyl, alkoxy, substituted alkoxy, and acyl. In certain instances, R³ is hydrogen. In certain instances, R³ is alkyl or substituted alkyl. In certain instances, R³ is alkyl, such as C₁-C₆ alkyl, including C₁-C₃ alkyl. In certain instances, R³ is methyl, ethyl, n-propyl, or isopropyl. In certain instances, R³ is methyl. In certain instances, R³ is alkenyl or substituted alkenyl. In certain instances, R³ is selected from alkynyl or substituted alkynyl. In certain instances, R³ is alkoxy or substituted alkoxy. In certain instances, R³ is acyl.

In formula I, R^{4a} is selected from hydrogen, C₁-C₃ alkyl, C5-C10 alkyl, and substituted alkyl. In certain instances, R^{4a} is hydrogen. In certain instances, R^{4a} is C₁-C₃ alkyl. In certain instances, R^{4a} is C₅-C₁₀ alkyl. In certain instances, R^{4a} is substituted alkyl. In certain instances, R^{4a} is methyl, ethyl, n-propyl, or isopropyl. In certain instances, R^{4a} is methyl.

In formula I, R⁵ is selected from hydrogen, alkyl, substituted alkyl, alkenyl, substituted alkenyl, alkynyl, substituted alkynyl, hydroxy, alkoxy, substituted alkoxy, acyloxy, thiol, acyl, amino, substituted amino, aminoacyl, acylamino, azido, carboxyl, carboxylalkyl, cyano, halogen, and nitro.

In certain instances, R⁵ is hydrogen. In certain instances, R⁵ is alkyl or substituted alkyl. In certain instances, R⁵ is alkenyl or substituted alkenyl. In certain instances, R⁵ is alkynyl or substituted alkynyl. In certain instances, R⁵ is hydroxy, alkoxy, substituted alkoxy, or acyloxy. In certain instances, R⁵ is thiol. In certain instances, R⁵ is acyl. In certain instances, R⁵ is amino, substituted amino, aminoacyl, acylamino, or azido. In certain instances, R⁵ is carboxyl or carboxylalkyl. In certain instances, R⁵ is cyano. In certain instances, R⁵ is nitro. In certain instances, R⁵ is halogen. In certain instances, R⁵ is fluoro. In certain instances, R⁵ is chloro. In certain instances, R⁵ is bromo.

In certain instances, formula I is the following formula:

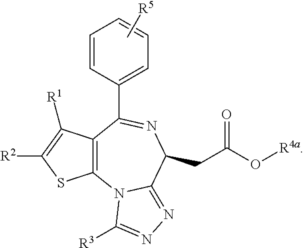

A particular compound of interest, and salts or solvates or stereoisomers thereof, includes:

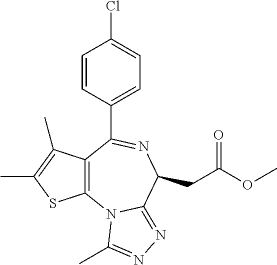

### (Methyl 2-((6S)-4-(4-chlorophenyl)-2,3,9-trimethyl-6H-thieno[3,2-f][1,2,4]triazolo[4,3-a][1,4]diazepin-6-yl)acetate)

Suitable HDAC inhibitors include hydroxamic acids (e.g., vorinostat (suberoylanilide hydroxamic acid, SAHA, Archin et al., AIDS Res Hum Retroviruses, 25(2): 207-12, 2009; Contreras et al. J Biol Chem, 284:6782-9, 2009), belinostat (PXD101), LAQ824; and panobinostat (LBH589); and benzamides (e.g., entinostat (MS-275), CI994; and mocetinostat (MGCD0103). Suitable HDAC inhibitors include butyric acid (including sodium butyrate and other salt forms), Valproic acid (including Mg valproate and other salt forms), suberoylanilide hydroxamic acid (SAHA), Vorinostat, Romidepsin (trade name Istodax), Panobinostat (LBH589), Belinostat (PXD101), Mocetinostat (MGCD0103), PCI-24781, Entinostat (MS-275), SB939, Resminostat (4SC-201); Givinostat (ITF2357), CUDC-101, AR-42, CHR-2845, CHR-3996, 4SC-202, sulforaphane, BML-210, M344, CI-994; CI-994 (Tacedinaline); BML-210; M344; MGCD0103 (Mocetinostat); and Tubastatin A. Additional suitable HDAC inhibitors are described in U.S. Pat. No. 7,399,787.

Suitable bryostatins include bryostatin-1; a bryostatin analog as described in U.S. Pat. No. 6,624,189; bryostatin-2; a bryostatin analog as described in U.S. Pat. No. 7,256,286; a bryostatin analog described in U.S. Patent Publication No. 20090270492; a bryostatin analog described in WO 2013/165592; etc.

In some cases, the second agent is a cell-permeable inhibitor of methyltransferase activity of a SMYD2 polypeptide, an ASH1L polypeptide, an SUV420H1 polypeptide and/or an SUV39H1 polypeptide as described in U.S. Patent Application Publication No. US2018-0002699A1, the disclosure of which is incorporated herein by reference in its entirety and for all purposes. In some cases, the second agent is a cell-permeable inhibitor of a Smyd2 polypeptide, where the second agent is a selective Smyd2 inhibitor. In some embodiments, a selective Smyd2 inhibitor does not substantially inhibit a Smyd1 polypeptide, a Smyd3 polypeptide, a Smyd4 polypeptide, or a Smyd5 polypeptide.

In some cases, the second agent is a cell-permeable inhibitor of methyltransferase activity of a Smyd2 polypeptide; and the second agent has an IC₅₀ of from about 0.001 μM to about 100 μM. In some cases, the second agent is a cell-permeable inhibitor of methyltransferase activity of a Smyd2 polypeptide; and the second agent has an IC₅₀ of from about 0.001 μM to about 100 μM. In some cases, the second agent is a cell-permeable inhibitor of methyltransferase activity of a Smyd2 polypeptide; and the second agent has an IC₅₀ of from about 0.001 μM to about 10 μM. In some cases, the second agent is a cell-permeable inhibitor of methyltransferase activity of a Smyd2 polypeptide; and the second agent has an IC₅₀ of from about 0.001 μM to about 1 μM. In some cases, the second agent is a cell-permeable inhibitor of methyltransferase activity of a Smyd2 polypeptide; and the second agent has an IC₅₀ of from about 0.001 μM to about 0.002 μM, from about 0.002 μM to about 0.00 μM, from about 0.003 μM to about 0.005 μM, from 0.005 μM to about 0.01 μM, from about 0.010 μM to about 0.01 μM, from about 0.015 μM to about 0.0 μM, from about 0.02 μM to about 0.0 μM, from about 0.05 μM to about 0.1 μM, from about 0.1 μM to about 0.5 μM, or from about 0.5 μM to about 1.0 μM. In some cases, the second agent is a cell-permeable inhibitor of methyltransferase activity of a Smyd2 polypeptide; and the second agent has an IC₅₀ of from about 1.0 μM to about 5 μM, from about 5 μM to about 10 μM, from about 10 μM to about 25 μM, from about 25 μM to about 50 μM, from about 50 μM to about 7 μM, or from about 75 μM to about 100 μM. In some cases, the second agent is a cell-permeable inhibitor of methyltransferase activity of a Smyd2 polypeptide; and the second agent has an IC₅₀ of from about 100 μM to about 1 nM. In some cases, the second agent is a cell-permeable inhibitor of methyltransferase activity of a Smyd2 polypeptide; and the second agent has an IC₅₀ of from about 1 nM to about 50 nM. In some cases, the second agent is a cell-permeable inhibitor of methyltransferase activity of a Smyd2 polypeptide; and the second agent has an IC₅₀ of from about 50 nM to about 100 nM.

An example of a suitable second agent is AZ505. AZ505 (N-cyclohexyl-3-((3,4-dichlorophenethyl)amino)-N-(2-((2-(5-hydroxy-3-oxo-3,4-dihydro-2H-benzo[b][1,4]oxazin-8-yl)ethyl)amino)ethyl)propanamide bis(2,2,2-trifluoroacetate)) is a selective Smyd2 inhibitor. Ferguson et al. (2011) Structure 19:1262. AZ505 has the following structure:

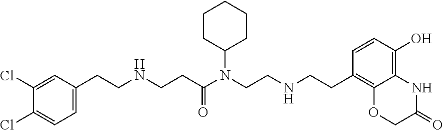 [SMILES: O=C1COc2c(CCNCCN(C(=O)CCNCCc3ccc(Cl)c(Cl)c3)C3CCCCC3)ccc(O)c2N1]

In some cases, it may be desirable to administer AZ505 in combination with a cell-permeability enhancer and/or administer an AZ505 derivative which has increased cell-permeability relative to AZ505. In some cases, it may be desirable to administer AZ505 as a conjugate with a PTD or CPP as described herein.

An example of a suitable second agent is LLY-507. LLY-507 is a potent inhibitor of Smyd2 with in vitro IC₅₀ less than 15 nm, and approximately 100-fold selectivity over other methyltransferases and other non-epigenetic targets. LLY-507 has the following structure:

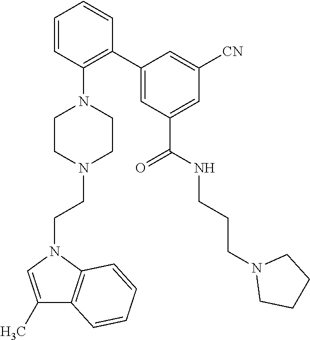 [SMILES: Cc1cn(CCN2CCN(c3ccccc3-c3cc(C#N)cc(C(=O)NCCCN4CCCC4)c3)CC2)c2ccccc12]

Another example of a suitable active agent is AZ506, also referred herein as “X1”, or a pharmaceutically acceptable derivative, e.g., salt thereof. AZ506 is a potent and selective bi-arylpiperazine, cell-permeable substrate competitive SMYD2 inhibitor with IC₅₀ 0.017 μM. AZ506 has the following structure:

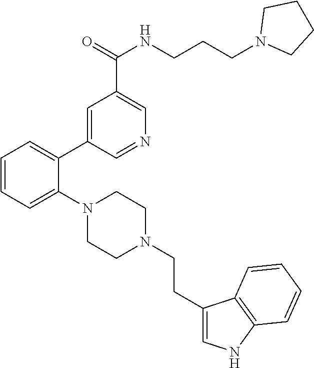 [SMILES: O=C(NCCCN1CCCC1)c1cncc(-c2ccccc2N2CCN(CCc3c[nH]c4ccccc34)CC2)c1]

An example of a suitable active agent is AZ391, or a pharmaceutically acceptable derivative, e.g., salt thereof. AZ391 is a potent and selective bi-arylpiperazine substrate competitive SMYD2 inhibitor with IC₅₀ 0.062 μM. AZ391 has the following structure:

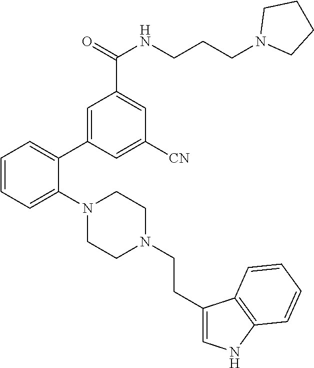 [SMILES: N#Cc1cc(C(=O)NCCCN2CCCC2)cc(-c2ccccc2N2CCN(CCc3c[nH]c4ccccc34)CC2)c1]

Combinations of two or more SMYD2 inhibitors can also be used in a method of the present disclosure.

An example of a suitable active agent is A-196 also known as Cyclopentyl-(6,7-dichloro-4-pyridin-4-yl-phthalazin-1-yl)-amine, or a pharmaceutically acceptable derivative, e.g., salt thereof. A-196 is a potent and selective inhibitor of SUV420H1 that inhibits the methylation of H4K20me. A-196 has the following structure:

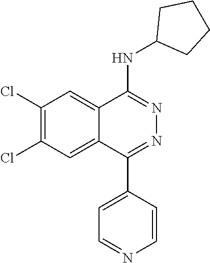 [SMILES: Clc1cc2c(NC3CCCC3)nnc(-c3ccncc3)c2cc1Cl]

An example of a suitable active agent is BIX-01294 also known as diazepin-quinazolin-amine derivative, or a pharmaceutically acceptable derivative, e.g., salt thereof. BIX-01294 is a SUV39H1 inhibitor that selectively impairs the generation of H3K9me2. BIX-01294 has the following structure:

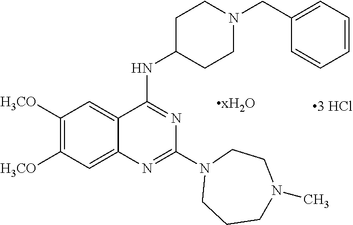 [SMILES: COc1cc2nc(N3CCCN(C)CC3)nc(NC3CCN(Cc4ccccc4)CC3)c2cc1OC]

An example of a suitable active agent is UNC0638 also known as 2-Cyclohexyl-N-(1-isopropylpiperidin-4-yl)-6-methoxy-7-(3-(pyrrolidin-1-yl)propoxy) quinazolin-4-amine, or a pharmaceutically acceptable derivative, e.g., salt thereof. UNC0638 is a selective inhibitor of SUV39H1. UNC0638 has the following structure:

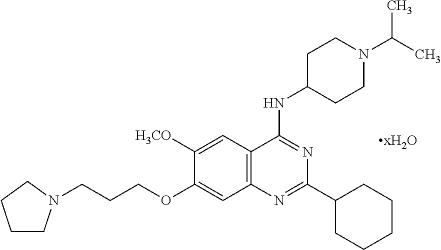 [SMILES: COc1cc2c(NC3CCN(C(C)C)CC3)nc(C3CCCCC3)nc2cc1OCCCN1CCCC1]

Combinations of two or more SUV39H1inhibitors can also be used in a method of the present disclosure.

In some embodiments, the co-administration of compounds results in synergism, and the combination is therefore a synergistic combination. As used herein, a “synergistic combination” or a “synergistic amount” of (i) a FOXO1 inhibitor that activates immunodeficiency virus transcription; and (ii) a second agent that activates immunodeficiency virus transcription is an amount that is more effective in activating immunodeficiency virus transcription when co-administered than the incremental increase that could be predicted or expected from a merely additive combination of (i) and (ii) when each is administered at the same dosage alone (not co-administered).

In some cases, a method of the present disclosure of treating an immunodeficiency virus infection in an individual in need thereof involves: a) administering to the individual an effective amount of AS1842856; and b) administering to the individual an effective amount of JQ1. In some cases, a method of the present disclosure of treating an immunodeficiency virus infection in an individual in need thereof involves: a) administering to the individual an effective amount of AS1842856; and b) administering to the individual an effective amount of SAHA. In some cases, a method of the present disclosure of treating an immunodeficiency virus infection in an individual in need thereof involves: a) administering to the individual an effective amount of AS1842856; and b) administering to the individual an effective amount of bryostatin or a bryostatin analog. In some cases, a method of the present disclosure of treating an immunodeficiency virus infection in an individual in need thereof involves: a) administering to the individual an effective amount of AS1842856; and b) administering to the individual an effective amount of an HDAC inhibitor. In some cases, a method of the present disclosure of treating an immunodeficiency virus infection in an individual in need thereof involves: a) administering to the individual an effective amount of AS1842856; and b) administering to the individual an effective amount of prostratin or a prostratin analog.

Combination Methods and Therapies—FOXO1 Inhibitor and/or ER Stress-Inducing Active Agent+Anti-Viral Agent

In some embodiments, the present disclosure provides a method of reactivating latent human immunodeficiency virus (HIV) integrated into the genome of a cell infected with HIV, the method including contacting the cell with a FOXO1 inhibitor and/or an ER stress-inducing agent (with or without a Ca²⁺-mobilizing agent) that reactivates latent HIV integrated into the genome of the cell. Alternatively, or in addition, to contacting the cell with at least one second agent that reactivates latent HIV as discussed above, the method may include contacting the cell with an effective amount of antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof.

In some embodiments, the antiretroviral drug is a nucleoside reverse transcriptase inhibitor selected from the group consisting of Zidovudine, Didanosine, Stavudine, Lamivudine, Abacavir, Tenofovir, Combivir, Trizivir, Emtricitabine, Truvada, Epzicom, and combinations thereof.

In some embodiments, the antiretroviral drug is a non-nucleoside reverse transcriptase inhibitor selected from the group consisting of Nevirapine, Delavirdine, Efavirenz, Etravirine, Rilpivirine, and combinations thereof.

In some embodiments, the antiretroviral drug is a protease inhibitor selected from the group consisting of Saquinavir, Indinavir, Ritonavir, Nelfinavir, Amprenavir, Lopinavir, Atazanavir, Fosamprenavir, Tipranavir, Darunavir, and combinations thereof.

In some embodiments, the antiretroviral drug is an entry inhibitor selected from the group consisting of Enfuvirtide, Maraviroc, and a combination thereof.

In some embodiments, the antiretroviral drug is an integrase inhibitor selected from the group consisting of Raltegravir, Elvitegravir, Dolutegravir, and combinations thereof.

The present disclosure provides a method of reducing the number of cells containing a latent human immunodeficiency virus in an individual, the method including administering to the individual an effective amount of a FOXO1 inhibitor and/or an ER stress-inducing agent (with or without a Ca²⁺-mobilizing agent) that reactivates latent HIV integrated into the genome of one or more cells in the individual.

Alternatively, or in addition, to administering to the individual at least one second agent that reactivates latent HIV as discussed above, the method may include administering to the individual an effective amount of antiretroviral drug, as discussed above and elsewhere herein.

The present disclosure provides a method of treating an immunodeficiency virus infection in an individual in need thereof involves: a) administering to the individual an effective amount of a FOXO1 inhibitor and/or an ER stress-inducing agent (with or without a Ca²⁺-mobilizing agent) that activates immunodeficiency virus transcription; and b) administering to the individual an effective amount of an agent that inhibits an immunodeficiency virus function. The immunodeficiency virus function can be selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

In some embodiments, a compound that is a FOXO1 inhibitor and/or an ER stress-inducing agent (with or without a Ca²⁺-mobilizing agent) (e.g., an agent that inhibits FOXO1 enzymatic activity and/or reduces the level of FOXO1 polypeptide in a cell) and that activates immunodeficiency virus transcription is administered in a combination therapy (i.e., co-administered) with: 1) one or more nucleoside reverse transcriptase inhibitors (e.g., Combivir, Epivir, Hivid, Retrovir, Videx, Zerit, Ziagen, etc.); 2) one or more non-nucleoside reverse transcriptase inhibitors (e.g., Rescriptor, Sustiva, Viramune, etc.); 3) one or more protease inhibitors (e.g., Agenerase, Crixivan, Fortovase, Invirase, Kaletra, Norvir, Viracept, etc.); 4) an anti-HIV agent such as a protease inhibitor and a nucleoside reverse transcriptase inhibitor; 5) an anti-HIV agent such as a protease inhibitor, a nucleoside reverse transcriptase inhibitor, and a non-nucleoside reverse transcriptase inhibitor; 6) an anti-HIV agent such as a protease inhibitor and a non-nucleoside reverse transcriptase inhibitor, and/or 7) an anti-viral (e.g., HIV) agent such as a protein kinase C (PKC) activator (e.g., prostratin). Other combinations of an effective amount of a FOXO1 inhibitor with one or more anti-HIV agents, such as one or more of a protease inhibitor, a nucleoside reverse transcriptase inhibitor, a non-nucleoside reverse transcriptase inhibitor, and a protein kinase C (PKC) activator are contemplated.

A PKC activator (e.g., prostratin ((1aR,1bS,4aR,7aS,7bR,8R,9aS)-4a,7b-dihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl)) can be administered in a separate formulation from a FOXO1 inhibitor. A PKC activator can be co-formulated with a FOXO1 inhibitor, and the co-formulation administered to an individual.

In some embodiments, the co-administration of compounds results in synergism, and the combination is therefore a synergistic combination. As used herein, a “synergistic combination” or a “synergistic amount” of (i) a FOXO1 inhibitor that activates immunodeficiency virus transcription and/or an ER stress-inducing agent (with or without a Ca²⁺-mobilizing agent) and (ii) an anti-viral agent (e.g., a nucleoside reverse transcriptase inhibitor, a non-nucleoside reverse transcriptase inhibitor, a protease inhibitor, an anti-HIV agent, a protein kinase C (PKC) activator, etc.) is an amount that is more effective in reducing immunodeficiency virus load when co-administered than the incremental increase that could be predicted or expected from a merely additive combination of (i) and (ii) when each is administered at the same dosage alone (not co-administered). As used herein, a “synergistic combination” or a “synergistic amount” of (i) a FOXO1 inhibitor and/or an ER stress-inducing agent (with or without a Ca²⁺-mobilizing agent) that activates immunodeficiency virus transcription and (ii) a second agent that activates latent immunodeficiency virus transcription, is an amount that is more effective in reactivating latent immunodeficiency virus transcription when co-administered than the incremental increase that could be predicted or expected from a merely additive combination of (i) and (ii) when each is administered at the same dosage alone (not co-administered).

Any of a variety of methods can be used to determine whether a treatment method is effective. For example, methods of determining whether the methods of the present disclosure are effective in reducing immunodeficiency virus (e.g., HIV) viral load, and/or treating an immunodeficiency virus (e.g., HIV) infection, are any known test for indicia of immunodeficiency virus (e.g., HIV) infection, including, but not limited to, measuring viral load, e.g., by measuring the amount of immunodeficiency virus (e.g., HIV) in a biological sample, e.g., using a polymerase chain reaction (PCR) with primers specific for an immunodeficiency virus (e.g., HIV) polynucleotide sequence; detecting and/or measuring a polypeptide encoded by an immunodeficiency virus (e.g., HIV), e.g., p24, gp120, reverse transcriptase, using, e.g., an immunological assay such as an enzyme-linked immunosorbent assay (ELISA) with an antibody specific for the polypeptide; and measuring the CD4⁺ T cell count in the individual.

### Formulations, Dosages, and Routes of Administration

In general, an active agent (e.g., a FOXO1 inhibitor, a FOXO1 activator, an ER stress-inducing agent, or an ER stress-inhibiting agent) is prepared in a pharmaceutically acceptable composition(s) for delivery to a host. In the context of reducing immunodeficiency virus transcription, the terms “active agent,” “drug,” “agent,” “therapeutic agent,” and the like are used interchangeably herein to refer to an agent that is a FOXO1 inhibitor and that activates latent immunodeficiency virus transcription.

Pharmaceutically acceptable carriers suitable for use with active agents (and optionally one or more additional therapeutic agents) may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising an active agent (and optionally one or more additional therapeutic agent) may also be lyophilized using means well known in the art, for subsequent reconstitution and use according to the invention.

### Formulations

An active agent is administered to an individual in need thereof in a formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. For the purposes of the following description of formulations, “active agent” includes an active agent as described above (e.g., a combination of two or more active agents as described above, which may be co-formulated), and optionally one or more additional therapeutic agent.

In a subject method, an active agent may be administered to the host using any convenient means capable of resulting in the desired degree of reduction of immunodeficiency virus transcription. Thus, an active agent can be incorporated into a variety of formulations for therapeutic administration. For example, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In an exemplary embodiment, an active agent is formulated as a gel, as a solution, or in some other form suitable for intravaginal administration. In a further exemplary embodiment, an active agent is formulated as a gel, as a solution, or in some other form suitable for rectal (e.g., intrarectal) administration.

In pharmaceutical dosage forms, an active agent may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, an active is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the active agent(s) in a composition as a solution in sterile water, normal saline or another pharmaceutically acceptable carrier.

Unit dosage forms for intravaginal or intrarectal administration such as syrups, elixirs, gels, and suspensions may be provided wherein each dosage unit, for example, teaspoonful, tablespoonful, tablet, unit gel volume, or suppository, contains a predetermined amount of the composition containing one or more active agents.

The term “unit dosage form,” as used herein, refers to physically discrete units suitable as unitary dosages for human and animal subjects, each unit containing a predetermined quantity of an active agent, calculated in an amount sufficient to produce the desired effect in association with a pharmaceutically acceptable diluent, carrier or vehicle. The specifications for a given active agent will depend in part on the particular compound employed and the effect to be achieved, and the pharmacodynamics associated with each compound in the host.

Other modes of administration will also find use with a method of the present disclosure. For instance, an active agent can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g. about 1% to about 2%.

An active agent can be administered in an injectable formulation. Typically, injectable compositions are prepared as liquid solutions or suspensions; solid forms suitable for solution in, or suspension in, liquid vehicles prior to injection may also be prepared. The preparation may also be emulsified, or the active ingredient encapsulated in liposome vehicles.

An active agent will in some embodiments be formulated for vaginal delivery. A subject formulation for intravaginal administration comprises an active agent formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

An active agent will in some embodiments be formulated for rectal delivery. A subject formulation for intrarectal administration comprises an active agent formulated as an intrarectal bioadhesive tablet, intrarectal bioadhesive microparticle, intrarectal cream, intrarectal lotion, intrarectal foam, intrarectal ointment, intrarectal paste, intrarectal solution, or intrarectal gel.

A subject formulation comprising an active agent includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

Although the dosage used will vary depending on the clinical goals to be achieved, a suitable dosage range of an active agent is one which provides up to about 1 mg to about 1000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, or from about 500 mg to about 1000 mg of an active agent can be administered in a single dose.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, a single dose of an active agent is administered. In other embodiments, multiple doses of an active agent are administered. Where multiple doses are administered over a period of time, an active agent is administered twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an active agent is administered at any of the aforementioned frequencies for one week, two weeks, one month, two months, six months, one year, or two years, or more, depending on various factors.

Where two different active agents are administered, a first active agent and a second active agent can be administered in separate formulations. A first active agent and a second active agent can be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

### Routes of Administration

An active agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, transdermal, subcutaneous, intradermal, topical application, intravenous, vaginal, nasal, and other parenteral routes of administration. In some embodiments, an active agent is administered via an intravaginal route of administration. In other embodiments, an active agent is administered via an intrarectal route of administration. Routes of administration may be combined, if desired, or adjusted depending upon the agent and/or the desired effect. The composition can be administered in a single dose or in multiple doses.

An active agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, vaginal, transdermal, subcutaneous, intramuscular, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

An active agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as the number of viral particles per unit blood. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally, such hosts are “mammals” or “mammalian,” where these terms are used broadly to describe organisms which are within the class mammalia, and primates (e.g., humans, chimpanzees, and monkeys), that are susceptible to immunodeficiency virus (e.g., HIV) infection. In many embodiments, the hosts will be humans.

### Kits, Containers, Devices, Delivery Systems

Kits with unit doses of one or more active agents as described herein, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the drugs in treating an immunodeficiency virus (e.g., an HIV) infection. Suitable active agents and unit doses are those described herein above.

In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

The present invention provides a delivery system comprising an active agent (e.g., a FOXO1 inhibitor and/or an ER stress-inducing agent (with or without a Ca²⁺-mobilizing agent); optionally also one or more additional therapeutic agents). In some embodiments, the delivery system is a delivery system that provides for injection of a formulation comprising an active agent subcutaneously, intravenously, or intramuscularly. In other embodiments, the delivery system is a vaginal or rectal delivery system.

In some embodiments, an active agent is packaged for oral administration. The present invention provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a delivery system of the present disclosure comprises an injection device. Exemplary, non-limiting drug delivery devices include injections devices, such as pen injectors, and needle/syringe devices. In some embodiments, the invention provides an injection delivery device that is pre-loaded with a formulation comprising an effective amount of a FOXO1 inhibitor and/or an ER stress-inducing agent (with or without a Ca²⁺-mobilizing agent). For example, a subject delivery device comprises an injection device pre-loaded with a single dose of a FOXO1 inhibitor and/or an ER stress-inducing agent (with or without a Ca²⁺-mobilizing agent). A injection device can be re-usable or disposable.

Pen injectors are well known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

The present invention provides a delivery system for vaginal or rectal delivery of an active agent to the vagina or rectum of an individual. The delivery system comprises a device for insertion into the vagina or rectum. In some embodiments, the delivery system comprises an applicator for delivery of a formulation into the vagina or rectum; and a container that contains a formulation comprising an active agent. In these embodiments, the container (e.g., a tube) is adapted for delivering a formulation into the applicator. In other embodiments, the delivery system comprises a device that is inserted into the vagina or rectum, which device includes an active agent. For example, the device is coated with, impregnated with, or otherwise contains a formulation comprising the active agent.

In some embodiments, the vaginal or rectal delivery system is a tampon or tampon-like device that comprises a subject formulation. Drug delivery tampons are known in the art, and any such tampon can be used in conjunction with a subject drug delivery system. Drug delivery tampons are described in, e.g., U.S. Pat. No. 6,086,909. If a tampon or tampon-like device is used, there are numerous methods by which an active agent can be incorporated into the device. For example, the drug can be incorporated into a gel-like bioadhesive reservoir in the tip of the device. Alternatively, the drug can be in the form of a powdered material positioned at the tip of the tampon. The drug can also be absorbed into fibers at the tip of the tampon, for example, by dissolving the drug in a pharmaceutically acceptable carrier and absorbing the drug solution into the tampon fibers. The drug can also be dissolved in a coating material which is applied to the tip of the tampon. Alternatively, the drug can be incorporated into an insertable suppository which is placed in association with the tip of the tampon.

In other embodiments, the drug delivery device is a vaginal or rectal ring. Vaginal or rectal rings usually consist of an inert elastomer ring coated by another layer of elastomer containing an active agent to be delivered. The rings can be easily inserted, left in place for the desired period of time (e.g., up to 7 days), then removed by the user. The ring can optionally include a third, outer, rate-controlling elastomer layer which contains no drug. Optionally, the third ring can contain a second drug for a dual release ring. The drug can be incorporated into polyethylene glycol throughout the silicone elastomer ring to act as a reservoir for drug to be delivered.

In other embodiments, a subject vaginal or rectal delivery system is a vaginal or rectal sponge. The active agent is incorporated into a silicone matrix which is coated onto a cylindrical drug-free polyurethane sponge, as described in the literature.

Pessaries, tablets, and suppositories are other examples of drug delivery systems which can be used, e.g., in carrying out a method of the present disclosure. These systems have been described extensively in the literature.

Bioadhesive microparticles constitute still another drug delivery system suitable for use in the present invention. This system is a multi-phase liquid or semi-solid preparation which does not seep from the vagina or rectum as do many suppository formulations. The substances cling to the wall of the vagina or rectum and release the drug over a period of time. Many of these systems were designed for nasal use but can be used in the vagina or rectum as well (e.g. U.S. Pat. No. 4,756,907). The system may comprise microspheres with an active agent; and a surfactant for enhancing uptake of the drug. The microparticles have a diameter of 10-100 μm and can be prepared from starch, gelatin, albumin, collagen, or dextran.

Another system is a container comprising a subject formulation (e.g., a tube) that is adapted for use with an applicator. The active agent is incorporated into creams, lotions, foams, paste, ointments, and gels which can be applied to the vagina or rectum using an applicator. Processes for preparing pharmaceuticals in cream, lotion, foam, paste, ointment and gel formats can be found throughout the literature. An example of a suitable system is a standard fragrance-free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal or rectal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

### Combination Therapy

In some embodiments, a FOXO1 inhibitor and/or an ER stress-inducing agent (with or without a Ca²⁺-mobilizing agent) is administered in combination therapy with one or more additional therapeutic agents. Suitable additional therapeutic agents include agents that inhibit one or more functions of an immunodeficiency virus; agents that treat or ameliorate a symptom of an immunodeficiency virus infection; agents that treat an infection that occurs secondary to an immunodeficiency virus infection; and the like. As noted above, suitable additional therapeutic agents include agents (other than a FOXO1 inhibitor and/or an ER stress-inducing agent (with or without a Ca²⁺-mobilizing agent) that reactivate latent immunodeficiency virus as described herein.

Therapeutic agents include, e.g., beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof. Anti-HIV agents are those in the preceding list that specifically target a function of one or more HIV proteins.

In some embodiments, a FOXO1 inhibitor and/or an ER stress-inducing agent (with or without a Ca²⁺-mobilizing agent) is administered in combination therapy with two or more anti-HIV agents. For example, a FOXO1 inhibitor can be administered in combination therapy with one, two, or three nucleoside reverse transcriptase inhibitors (e.g., Combivir, Epivir, Hivid, Retrovir, Videx, Zerit, Ziagen, etc.). A FOXO1 inhibitor and/or an ER stress-inducing agent (with or without a Ca²⁺-mobilizing agent) can be administered in combination therapy with one or two non-nucleoside reverse transcriptase inhibitors (e.g., Rescriptor, Sustiva, Viramune, etc.). A FOXO1 inhibitor and/or an ER stress-inducing agent (with or without a Ca²⁺-mobilizing agent) can be administered in combination therapy with one or two protease inhibitors (e.g., Agenerase, Crixivan, Fortovase, Invirase, Kaletra, Norvir, Viracept, etc.). A FOXO1 inhibitor and/or an ER stress-inducing agent (with or without a Ca²⁺-mobilizing agent) can be administered in combination therapy with a protease inhibitor and a nucleoside reverse transcriptase inhibitor. A FOXO1 inhibitor and/or an ER stress-inducing agent (with or without a Ca²⁺-mobilizing agent) can be administered in combination therapy with a protease inhibitor, a nucleoside reverse transcriptase inhibitor, and a non-nucleoside reverse transcriptase inhibitor. A FOXO1 inhibitor and/or an ER stress-inducing agent (with or without a Ca²⁺-mobilizing agent) can be administered in combination therapy with a protease inhibitor and a non-nucleoside reverse transcriptase inhibitor. Other combinations of a FOXO1 inhibitor and/or an ER stress-inducing agent (with or without a Ca²⁺-mobilizing agent) with one or more of a protease inhibitor, a nucleoside reverse transcriptase inhibitor, and a non-nucleoside reverse transcriptase inhibitor are contemplated.

In some embodiments, a treatment method of the present disclosure involves administering: a) a FOXO1 inhibitor and/or an ER stress-inducing agent (with or without a Ca²⁺-mobilizing agent); and b) an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

In some embodiments, a subject treatment method involves administering: a) a FOXO1 inhibitor and/or an ER stress-inducing agent (with or without a Ca²⁺-mobilizing agent); and b) an HIV inhibitor, where suitable HIV inhibitors include, but are not limited to, one or more nucleoside/nucleotide reverse transcriptase inhibitors (NRTIs), non-nucleoside reverse transcriptase inhibitors (NNRTIs), protease inhibitors (PIs), fusion inhibitors, integrase inhibitors, chemokine receptor (e.g., CXCR4, CCR5) inhibitors, and hydroxyurea.

Nucleoside reverse transcriptase inhibitors include, but are not limited to, abacavir (ABC; ZIAGEN™), didanosine (dideoxyinosine (ddI); VIDEX™), lamivudine (3TC; EPIVIR™), stavudine (d4T; ZERIT™, ZERIT XR™), zalcitabine (dideoxycytidine (ddC); HMD™), zidovudine (ZDV, formerly known as azidothymidine (AZT); RETROVIR™), abacavir, zidovudine, and lamivudine (TRIZIVIR™), zidovudine and lamivudine (COMBIVIR™), and emtricitabine (EMTRIVA™). Nucleotide reverse transcriptase inhibitors include tenofovir disoproxil fumarate (VIREAD™). Non-nucleoside reverse transcriptase inhibitors for HIV include, but are not limited to, nevirapine (VIRAMUNE™), delavirdine mesylate (RESCRIPTOR™), and efavirenz (SUSTIVA™).

Protease inhibitors (PIs) for treating HIV infection include amprenavir (AGENERASE™), saquinavir mesylate (FORTOVASE™, INVIRASE™), ritonavir (NORVIR™), indinavir sulfate (CRIXIVAN™), nelfmavir mesylate (VIRACEPT™), lopinavir and ritonavir (KALETRA™), atazanavir (REYATAZ™), and fosamprenavir (LEXIVA™).

Fusion inhibitors prevent fusion between the virus and the cell from occurring, and therefore, prevent HIV infection and multiplication. Fusion inhibitors include, but are not limited to, enfuvirtide (FUZEON™), Lalezari et al., New England J. Med., 348:2175-2185 (2003); and maraviroc (SELZENTRY™, Pfizer).

An integrase inhibitor blocks the action of integrase, preventing HIV-1 genetic material from integrating into the host DNA, and thereby stopping viral replication. Integrase inhibitors include, but are not limited to, raltegravir (ISENTRESS™, Merck); and elvitegravir (GS 9137, Gilead Sciences).

Maturation inhibitors include, e.g., bevirimat (3β-(3-carboxy-3-methyl-butanoyloxy) lup-20(29)-en-28-oic acid); and Vivecon (MPC9055).

In some embodiments, a subject treatment method involves administering: a) a FOXO1 inhibitor and/or an ER stress-inducing agent (with or without a Ca²⁺-mobilizing agent); and b) one or more of: (1) an HIV protease inhibitor selected from amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, R00334649, KNI-272, DPC-681, DPC-684, GW640385X, DG17, PPL-100, DG35, and AG 1859; (2) an HIV non-nucleoside inhibitor of reverse transcriptase selected from capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirene), efavirenz, BILR 355 BS, VRX 840773, UK-453061, and RDEA806; (3) an HIV nucleoside inhibitor of reverse transcriptase selected from zidovudine, emtricitabine, didanosine, stavudine, zalcitabine, lamivudine, abacavir, amdoxovir, elvucitabine, alovudine, MIV-210, racivir, D-d4FC, emtricitabine, phosphazide, fozivudine tidoxil, apricitibine (AVX754), amdoxovir, KP-1461, and fosalvudine tidoxil (formerly HDP 99.0003); (4) an HIV nucleotide inhibitor of reverse transcriptase selected from tenofovir and adefovir; (5) an HIV integrase inhibitor selected from curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-538158, GSK2656157364735C, BMS-707035, MK-2048, and BA 011; (6) a gp41 inhibitor selected from enfuvirtide, sifuvirtide, FB006M, and TRI-1144; (7) a CXCR4 inhibitor, such as AMD-070; (8) an entry inhibitor, such as SPO1A; (9) a gp120 inhibitor, such as BMS-488043 and/or BlockAide/CR; (10) a G6PD and NADH-oxidase inhibitor, such as immunitin; (11) a CCR5 inhibitors selected from the group consisting of aplaviroc, vicriviroc, maraviroc, PRO-140, INCB15050, PF-232798 (Pfizer), and CCR5 mAb004; (12) another drug for treating HIV selected from BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDXO10 (ipilimumab), PBS119, ALG 889, and PA-1050040 (PA-040); (13) any combinations or mixtures of the above.

As further examples, in some embodiments, a subject treatment method involves administering: a) a FOXO1 inhibitor and/or an ER stress-inducing agent (with or without a Ca²⁺-mobilizing agent); and b) one or more of: i) amprenavir (Agenerase; (3S)-oxolan-3-yl N-[(2S,3R)-3-hydroxy-4-[N-(2-methylpropyl)(4-aminobenzene)sulfonamido]-1-phenylbutan-2-yl]carbamate) in an amount of 600 mg or 1200 mg twice daily; ii) tipranavir (Aptivus; N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide) in an amount of 500 mg twice daily; iii) idinavir (Crixivan; (2S)-1-[(2S,4R)-4-benzyl-2-hydroxy-4-{[(1S,2R)-2-hydroxy-2,3-dihydro-1H-inden-1-yl]carbamoyl}butyl]-N-tert-butyl-4-(pyridin-3-ylmethyl)piperazine-2-carboxamide) in an amount of 800 mg three times daily; iv) saquinavir (Invirase; 2S)—N-[(2S,3R)-4-[(3S)-3-(tert-butylcarbamoyl)-decahydroisoquinolin-2-yl]-3-hydroxy-1-phenylbutan-2-yl]-2-(quinolin-2-ylformamido)butanediamide) in an amount of 1,000 mg twice daily; v) lopinavir and ritonavir (Kaleta; where lopinavir is 2S)—N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide; and ritonavir is 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate) in an amount of 133 mg twice daily; vi) fosamprenavir (Lexiva; {[(2R,3S)-1-[N-(2-methylpropyl)(4-aminobenzene)sulfonamido]-3-({[(3S)-oxolan-3-yloxy]carbonyl}amino)-4-phenylbutan-2-yl]oxy}phosphonic acid) in an amount of 700 mg or 1400 mg twice daily); vii) ritonavir (Norvir) in an amount of 600 mg twice daily; viii) nelfinavir (Viracept; (3S,4aS,8aS)—N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide) in an amount of 750 mg three times daily or in an amount of 1250 mg twice daily; ix) Fuzeon (Acetyl-YTSLIHSLIEESQNQ QEKNEQELLELDKWASLWNWF-amide (SEQ ID NO:21)) in an amount of 90 mg twice daily; x) Combivir in an amount of 150 mg lamivudine (3TC; 2′,3′-dideoxy-3′-thiacytidine) and 300 mg zidovudine (AZT; azidothymidine) twice daily; xi) emtricitabine (Emtriva; 4-amino-5-fluoro-1-[(2R,5S)-2-(hydroxymethyl)-1,3-oxathiolan-5-yl]-1,2-dihydropyrimidin-2-one) in an amount of 200 mg once daily; xii) Epzicom in an amount of 600 mg abacavir (ABV; {(1S,4R)-4-[2-amino-6-(cyclopropylamino)-9H-purin-9-yl]cyclopent-2-en-1-yl}methanol) and 300 mg 3TC once daily; xiii) zidovudine (Retrovir; AZT or azidothymidine) in an amount of 200 mg three times daily; xiv) Trizivir in an amount of 150 mg 3TC and 300 mg ABV and 300 mg AZT twice daily; xv) Truvada in an amount of 200 mg emtricitabine and 300 mg tenofovir ((([(2R)-1-(6-amino-9H-purin-9-yl)propan-2-yl]oxy)methyl)phosphonic acid) once daily; xvi) didanosine (Videx; 2′,3′-dideoxyinosine) in an amount of 400 mg once daily; xvii) tenofovir (Viread) in an amount of 300 mg once daily; xviii) abacavir (Ziagen) in an amount of 300 mg twice daily; xix) atazanavir (Reyataz; methyl N-[(1S)-1-{[(2S,3S)-3-hydroxy-4-[(2S)-2-[(methoxycarbonyl)amino]-3,3-dimethyl-N′-{[4-(pyridin-2-yl)phenyl]methyl}butanehydrazido]-1-phenylbutan-2-yl]carbamoyl}-2,2-dimethylpropyl]carbamate) in an amount of 300 mg once daily or 400 mg once daily; xx) lamivudine (Epivir) in an amount of 150 mg twice daily; xxi) stavudine (Zerit; 2′-3′-didehydro-2′-3′-dideoxythymidine) in an amount of 40 mg twice daily; xxii) delavirdine (Rescriptor; N-[2-({4-[3-(propan-2-ylamino)pyridin-2-yl]piperazin-1-yl}carbonyl)-1H-indol-5-yl]methanesulfonamide) in an amount of 400 mg three times daily; xxiii) efavirenz (Sustiva; (4S)-6-chloro-4-(2-cyclopropylethynyl)-4-(trifluoromethyl)-2,4-dihydro-1H-3,1-benzoxazin-2-one) in an amount of 600 mg once daily); xxiv) nevirapine (Viramune; 11-cyclopropyl-4-methyl-5,11-dihydro-6H-dipyrido[3,2-b:2′,3′-e][1,4]diazepin-6-one) in an amount of 200 mg twice daily); xxv) bevirimat; and xxvi) Vivecon.

In some embodiments, a subject treatment method involves administering: a) a FOXO1 inhibitor and/or an ER stress-inducing agent (with or without a Ca²⁺-mobilizing agent); and b) a PKC activator. An example of a suitable PKC activator is prostratin ((1aR,1bS,4aR,7aS,7bR,8R,9aS)-4a,7b-dihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl). The PKC activator can be administered in a separate formulation from a FOXO1 inhibitor and/or an ER stress-inducing agent (with or without a Ca²⁺-mobilizing agent). A PKC activator can be co-formulated with a FOXO1 inhibitor and/or an ER stress-inducing agent (with or without a Ca²⁺-mobilizing agent), and the co-formulation administered to an individual. The present disclosure provides a kit comprising a PKC activator in a first container; and a FOXO1 inhibitor and/or an ER stress-inducing agent (with or without a Ca²⁺-mobilizing agent) in a second container.

### Subjects Suitable for Treatment

The methods of the present disclosure are suitable for treating individuals who have an immunodeficiency virus infection, e.g., who have been diagnosed as having an immunodeficiency virus infection.

The methods of the present disclosure are suitable for treating individuals who have an HIV infection (e.g., who have been diagnosed as having an HIV infection), and individuals who are at risk of contracting an HIV infection. Such individuals include, but are not limited to, individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals. Individuals suitable for treatment include individuals infected with, or at risk of becoming infected with, HIV-1 and/or HIV-2 and/or HIV-3, or any variant thereof.

Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of reactivating latent human immunodeficiency virus (HIV) integrated into the genome of a cell infected with HIV, the method comprising contacting the cell with a FOXO1 inhibitor that reactivates latent HIV integrated into the genome of the cell, wherein the contacting takes place in the absence of an exogenously supplied immunodeficiency virus immunogen.
2. The method of 1, wherein the FOXO1 is a polypeptide comprising an amino acid sequence having at least 95% amino acid sequence identity to the amino acid sequence set forth in FIG. 11.
3. The method of 1 or 2, comprising contacting the cell with at least one second agent that reactivates latent HIV.
4. The method of 3, comprising contacting the cell with a synergistically effective amount of the at least one second agent.
5. The method of 3, wherein the at least one second agent is a histone deacetylase (HDAC) inhibitor, a protein kinase C (PKC) activator, or a bromodomain inhibitor.
6. The method of 3, wherein the at least one second agent is a Smyd2 inhibitor.
7. The method of 5, wherein the HDAC inhibitor is suberoylanilidehydroxamic (SAHA), romidepsin, or sodium butyrate.
8. The method of 5, wherein the PKC activator is prostratin, bryostatin, a chemical analog of prostratin, or a chemical analog of bryostatin.
9. The method of 5, wherein the bromodomain inhibitor is JQ1.
10. The method of any one of 1-9, comprising contacting the cell with an effective amount of antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof.
11. The method of 10, wherein the antiretroviral drug is a nucleoside reverse transcriptase inhibitor selected from the group consisting of Zidovudine, Didanosine, Stavudine, Lamivudine, Abacavir, Tenofovir, Combivir, Trizivir, Emtricitabine, Truvada, Epzicom, and combinations thereof.
12. The method of 10, wherein the antiretroviral drug is a non-nucleoside reverse transcriptase inhibitor selected from the group consisting of Nevirapine, Delavirdine, Efavirenz, Etravirine, Rilpivirine, and combinations thereof.
13. The method of 10, wherein the antiretroviral drug is a protease inhibitor selected from the group consisting of Saquinavir, Indinavir, Ritonavir, Nelfinavir, Amprenavir, Lopinavir, Atazanavir, Fosamprenavir, Tipranavir, Darunavir, and combinations thereof.
14. The method of 10, wherein the antiretroviral drug is an entry inhibitor selected from the group consisting of Enfuvirtide, Maraviroc, and a combination thereof.
15. The method of 10, wherein the antiretroviral drug is an integrase inhibitor selected from the group consisting of Raltegravir, Elvitegravir, Dolutegravir, and combinations thereof.
16. A method of reducing the number of cells containing a latent human immunodeficiency virus in an individual, the method comprising administering to the individual an effective amount of a FOXO1 inhibitor that reactivates latent HIV integrated into the genome of one or more cells in the individual, wherein the administering does not comprise administering an immunodeficiency virus immunogen to the individual.
17. The method of 16, wherein said administering steps is by a vaginal route of administration, by a rectal route of administration, by an oral route of administration, or by an intravenous route of administration.
18. The method of 16, wherein said administering is effective to reduce the number of cells containing a latent human immunodeficiency virus in the individual by at least 20%.
19. The method of any one of 16-18, comprising administering to the individual at least one second agent that reactivates latent HIV.
20. The method of 19, comprising administering to the individual a synergistically effective amount of the at least one second agent.
21. The method of 19, wherein the at least one second agent is a histone deacetylase (HDAC) inhibitor, a protein kinase C (PKC) activator, or a bromodomain inhibitor.
22. The method of 19, wherein the at least one second agent is a Smyd2 inhibitor.
23. The method of 21, wherein the HDAC inhibitor is suberoylanilidehydroxamic (SAHA), romidepsin, or sodium butyrate.
24. The method of 21, wherein the PKC activator is prostratin, bryostatin, a chemical analog of prostratin, or a chemical analog of bryostatin.
25. The method of 21, wherein the bromodomain inhibitor is JQ1.
26. The method of any one of 16-25, comprising contacting the cell with an effective amount of antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof.
27. The method of 26, wherein the antiretroviral drug is a nucleoside reverse transcriptase inhibitor selected from the group consisting of Zidovudine, Didanosine, Stavudine, Lamivudine, Abacavir, Tenofovir, Combivir, Trizivir, Emtricitabine, Truvada, Epzicom, and combinations thereof.
28. The method of 26, wherein the antiretroviral drug is a non-nucleoside reverse transcriptase inhibitor selected from the group consisting of Nevirapine, Delavirdine, Efavirenz, Etravirine, Rilpivirine, and combinations thereof.
29. The method of 26, wherein the antiretroviral drug is a protease inhibitor selected from the group consisting of Saquinavir, Indinavir, Ritonavir, Nelfinavir, Amprenavir, Lopinavir, Atazanavir, Fosamprenavir, Tipranavir, Darunavir, and combinations thereof.
30. The method of 26, wherein the antiretroviral drug is an entry inhibitor selected from the group consisting of Enfuvirtide, Maraviroc, and a combination thereof.
31. The method of 26, wherein the antiretroviral drug is an integrase inhibitor selected from the group consisting of Raltegravir, Elvitegravir, Dolutegravir, and combinations thereof.
32. A method of treating a human immunodeficiency virus (HIV) infection in an individual, the method comprising:
   administering to the individual an effective amount of a first active agent, wherein the first active agent is a FOXO1 inhibitor that reactivates latent HIV integrated into the genome of a cell in the individual; and
   administering to the individual an effective amount of a second active agent, wherein the second active agent inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity, and wherein the administering to the individual of an effective amount of a first active agent does not comprise administering an immunodeficiency virus immunogen to the individual.
33. The method of 32, wherein one or both of said administering steps is by a vaginal route of administration, by a rectal route of administration, by an oral route of administration, or by an intravenous route of administration.
34. The method of 32 or 33, comprising administering to the individual at least one second agent that reactivates latent HIV.
35. The method of 34, wherein the at least one second agent is a histone deacetylase (HDAC) inhibitor, a protein kinase C (PKC) activator, or a bromodomain inhibitor.
36. The method of 35, wherein the HDAC inhibitor is suberoylanilidehydroxamic (SAHA), romidepsin, or sodium butyrate.
37. The method of 35, wherein the PKC activator is prostratin, bryostatin, a chemical analog of prostratin, or a chemical analog of bryostatin.
38. The method of 35, wherein the bromodomain inhibitor is JQ1.
39. A method of treating a human immunodeficiency virus (HIV) infection in an individual, the method comprising:
   administering to the individual an effective amount of a first active agent, wherein the first active agent is a FOXO1 inhibitor that reactivates latent HIV integrated into the genome of a cell in the individual; and
   administering to the individual an effective amount of an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof, and wherein the administering to the individual of an effective amount of a first active agent does not comprise administering an immunodeficiency virus immunogen to the individual.
40. The method of 39, wherein one or both of said administering steps is by a vaginal route of administration, by a rectal route of administration, by an oral route of administration, or by an intravenous route of administration.
41. The method of 39 or 40, comprising administering to the individual at least one second agent that reactivates latent HIV.
42. The method of 41, wherein the at least one second agent is a histone deacetylase (HDAC) inhibitor, a protein kinase C (PKC) activator, or a bromodomain inhibitor.
43. The method of 42, wherein the HDAC inhibitor is suberoylanilidehydroxamic (SAHA), romidepsin, or sodium butyrate.
44. The method of 42, wherein the PKC activator is prostratin, bryostatin, a chemical analog of prostratin, or a chemical analog of bryostatin.
45. The method of 42, wherein the bromodomain inhibitor is JQ1.
46. The method of any one of 39-45, wherein the antiretroviral drug is a nucleoside reverse transcriptase inhibitor selected from the group consisting of Zidovudine, Didanosine, Stavudine, Lamivudine, Abacavir, Tenofovir, Combivir, Trizivir, Emtricitabine, Truvada, Epzicom, and combinations thereof.
47. The method of any one of 39-45, wherein the antiretroviral drug is a non-nucleoside reverse transcriptase inhibitor selected from the group consisting of Nevirapine, Delavirdine, Efavirenz, Etravirine, Rilpivirine, and combinations thereof.
48. The method of any one of 39-45, wherein the antiretroviral drug is a protease inhibitor selected from the group consisting of Saquinavir, Indinavir, Ritonavir, Nelfinavir, Amprenavir, Lopinavir, Atazanavir, Fosamprenavir, Tipranavir, Darunavir, and combinations thereof.
49. The method of any one of 39-45, wherein the antiretroviral drug is an entry inhibitor selected from the group consisting of Enfuvirtide, Maraviroc, and a combination thereof.
50. The method of any one of 39-45, wherein the antiretroviral drug is an integrase inhibitor selected from the group consisting of Raltegravir, Elvitegravir, Dolutegravir, and combinations thereof.
51. A drug delivery device comprising:
   a) a first container comprising a FOXO1 inhibitor that reactivates latent immunodeficiency virus transcription; and
   b) a second container comprising an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.
52. The device of 51, wherein the first and second containers are syringes, vials, or ampules.
53. A drug delivery device comprising:
   a) a first container comprising a FOXO1 inhibitor that reactivates latent immunodeficiency virus transcription; and
   b) a second container comprising an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof.
54. The device of 53, wherein the first and second containers are syringes, vials, or ampules.
55. The device of 53 or 54, wherein the antiretroviral drug is a nucleoside reverse transcriptase inhibitor selected from the group consisting of Zidovudine, Didanosine, Stavudine, Lamivudine, Abacavir, Tenofovir, Combivir, Trizivir, Emtricitabine, Truvada, Epzicom, and combinations thereof.
56. The device of 53 or 54, wherein the antiretroviral drug is a non-nucleoside reverse transcriptase inhibitor selected from the group consisting of Nevirapine, Delavirdine, Efavirenz, Etravirine, Rilpivirine, and combinations thereof.
57. The device of 53 or 54, wherein the antiretroviral drug is a protease inhibitor selected from the group consisting of Saquinavir, Indinavir, Ritonavir, Nelfinavir, Amprenavir, Lopinavir, Atazanavir, Fosamprenavir, Tipranavir, Darunavir, and combinations thereof.
58. The device of 53 or 54, wherein the antiretroviral drug is an entry inhibitor selected from the group consisting of Enfuvirtide, Maraviroc, and a combination thereof.
59. The device of 53 or 54, wherein the antiretroviral drug is an integrase inhibitor selected from the group consisting of Raltegravir, Elvitegravir, Dolutegravir, and combinations thereof.
60. The method of any one of 1-50, or the device of any one of 51-59, wherein the FOXO1 inhibitor is a small molecule FOXO1 inhibitor.
61. The method of any one of 1-50, or the device of any one of 51-59, wherein the FOXO1 inhibitor is AS1842856 (5-amino-7-(cyclohexylamino)-1-ethyl-6-fluoro-4-oxo-1,4-dihydro-quinoline-3-carboxylic acid) or a pharmaceutically acceptable salt thereof.
62. The method of any one of 1-50, or the device of any one of 51-59, wherein the FOXO1 inhibitor is an siNA, or a nucleic acid encoding an siNA.
63. A method of reactivating latent human immunodeficiency virus (HIV) integrated into the genome of a cell infected with HIV, the method comprising contacting the cell with an endoplasmic reticulum (ER) stress-inducing agent that reactivates latent HIV integrated into the genome of the cell.
64. The method of 63, wherein the ER stress-inducing agent is fenretinide or bortezomib.
65. The method of 63 or 64, wherein the method comprises contacting the cell with a Ca²⁺-mobilizing agent.
66. The method of 65, wherein the Ca²⁺-mobilizing agent is ionomycin.
67. The method of any one of 63-66, comprising contacting the cell with at least one second agent that reactivates latent HIV.
68. The method of 67, wherein the at least one second agent is a histone deacetylase (HDAC) inhibitor, a protein kinase C (PKC) activator, or a bromodomain inhibitor.
69. The method of 67 wherein the HDAC inhibitor is suberoylanilidehydroxamic (SAHA), romidepsin, or sodium butyrate.
70. The method of 67, wherein the PKC activator is prostratin, bryostatin, a chemical analog of prostratin, or a chemical analog of bryostatin.
71. The method of 67, wherein the bromodomain inhibitor is JQ1.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like. 72.

### Materials and Methods

### Cell Lines, Primary Cells, HIV Patients and Drug Treatments

J-Lat cell lines (clones A2, A72, 6.3, 11.1 14.8 and 5A8) were originally generated from male Jurkat cells. J-Lat cells were cultured at 37° C. in RPMI supplemented with 10% FBS, 1% L-glutamine and 1% penicillin-streptomycin. K562 cells (ATCC) were maintained in appropriate volume of RPMI medium supplemented with 10% fetal bovine serum (Serum Plus—II, Sigma MO, USA), 100 units/mL penicillin and 100 units/mL streptomycin (Corning, VA, USA). 293T cells were obtained from ATCC and cultured in DMEM supplemented with 10% FBS, 1% glutamine, and 1% penicillin-streptomycin at 37° C., 5% CO2. Short-term passages (<15) were used for all experiments. Primary CD4⁺ T cells were isolated via negative selection from peripheral blood mononuclear cells (PBMCs). Resting CD4⁺ T lymphocytes were enriched by negative depletion with an EasySep Human CD4⁺ T Cell Isolation Kit (Stemcell Technologies, Canada). Primary CD4⁺ T cells were cultured at 37° C. in RPMI supplemented with 10% FBS, 1% L-glutamine and 1% penicillin-streptomycin. This study sampled HIV-infected participants from the Zuckerberg San Francisco General Hospital clinic-based SCOPE cohorts. The UCSF Committee on Human Research approved this study, and participants gave informed, written consent before enrollment.

Cells were treated with the following compounds: FOXO inhibitor/AS1842856 (344355, Calbiochem); TNFα (300-01A, PeproTech); Raltegravir (CDS023737, Sigma-Aldrich); Prostratin (P4462, LC Laboratories); PHA-M (10576015, Sigma-Aldrich); IL-2 (12644, Sigma-Aldrich); αCD3 (Tonbo); αCD28 (Tonbo); PMA (P8139, Sigma-Aldrich); Ionomycin (I0634, Sigma-Aldrich); PERK inhibitor II/GSK2656157 (504651, Sigma-Aldrich); GCN2 inhibitor/A-92 (2720, Axon Medchem); Imidazolo-oxindole PKR inhibitor C16 (I9785, Sigma-Aldrich); Cyclosporin A (C3662, Sigma-Aldrich); Thapsigargin (T9033, Sigma-Aldrich); Brefeldin A 1000× Solution (00-4506-51, ThermoFisher Scientific); Fenretinide (17688, Cayman Chemicals).

### RNAseq+IPA

RNA was prepared from CD4⁺ T cells using the QIAgen RNeasy Plus Kit. The Gladstone Institutes Genomics Core carried out the downstream processing of the RNA samples. Strand-specific cDNA libraries were prepared using the Nugen Ovation kit (Nugen) and the libraries were deep sequenced on NextSeq 500 using paired-end 200 pb sequencing. RNA-seq analysis was done using the Illumina RNAexpress application v 1.1.0. Pathway analysis was carried out using Ingenuity Pathway Analysis and the Gene Ontology AmiGO Term Enrichment tool. For the Ingenuity analysis, the entire dataset was uploaded, and a two-direction analysis carried using filters to restrict the analysis to differentially expressed genes as above. For Gene Ontology, individual lists of genes were uploaded and analyzed separately.

### RNA Extraction, RT and Quantitative RT-PCR

Total RNA from samples was extracted using the Direct-zol RNA kit (R2060; Zymogen). cDNA was generated using 500 ng for CD4⁺ T cells or 1000 ng of total RNA for J-Lats with Superscript III Reverse transcription (18080-044; ThermoFisher) and oligo(dT) (18418-012; ThermoFisher). Quantitative RT-PCR was carried out using Maxima SYBR Green qPCR Master Mix (Thermo Scientific) on SDS 2.4 software (Applied Biosystems) in a total volume of 12 μL. The SYBR green qPCR reactions contained 5 μl of 2× Maxima SYBR green/Rox qPCR Master Mix (K0221; ThermoFisher), 5 μl of diluted cDNA, and 1 nmol of both forward and reverse primers. The reactions were run using the following conditions: 50° C. for 2 min and 95° C. for 10 min, followed by 40 cycles of 95° C. for 5 s and 62° C. for 30 s. See Table 1 below for a qRT-PCR primer list. Primer efficiencies were around 100%. Dissociation curve analysis was performed after the end of the PCR to confirm the presence of a single and specific product. All qPCRs were independently repeated at least three times, averaged and compared using standard deviation (±SD).

TABLE 1

| | qRT-PCR forward | qRT-PCR reverse |
|---|---|---|
| Gene | primers (5′-3′) | primers (5′-3′) |
| RPL13A | CCTGGAGGAG | TTGAGGACCT |
| | AAGAGGAAAG | CTGTGTATTT |
| | AGA | GTCAA |
| PYCR1 | TTTCTGCTCT | ACCACAATGT |
| | CAGGAAGATG | GTCTGTCCTC |
| YPEL4 | CCAAGACTTT | CGTTGACCAC |
| | CCGCAGCTAT | GGAGTTAAAC |
| | C | AG |
| SDK2 | TCAAGTGGCT | GCACGATGCA |
| | CCACAACAAC | ACGGTAAAAG |
| | A | C |
| MARS | CACAGCTTGA | CGGGATCTTG |
| | GTCGTCAGAA | CAGTAATGGG |
| | C | TAT |
| VARS | AGAAAGGTGA | CTGCTGAGAG |
| | CCGGATTTAC | TTTAGGGTCC |
| | CA | A |
| GARS | ATGGAGGTGT | CTGTTCCTCT |
| | TAGTGGTCTG | TGGATAAAGT |
| | T | GCT |
| PSAT1 | TGCCGCACTC | GCAATTCCCG |
| | AGTGTTGTTA | CACAAGATTC |
| | G | T |
| PHGDH | CACGACAGGC | CTTCCGTAAA |
| | TTGCTGAATG | CACGTCCAGT |
| | A | G |
| PSPH | GAGGACGCGG | GGTTGCTCTG |
| | TGTCAGAAAT | CTATGAGTCT |
| | | CT |
| SHMT2 | TGATTCCCTC | TTTCCGGTAG |
| | GCCTTTCAAG | AAGATGAGCC |
| | C | C |
| MTHFD1L | CTGCCTTCAA | TTTCCTGCAT |
| | GCCGGTTCTT | CAAGTTGTCG |
| | | T |
| SLC7A5 | GGAAGGGTGA | TAATGCCAGC |
| | TGTGTCCAAT | ACAATGTTCC |
| | C | C |
| SLC3A2 | TGAATGAGTT | GTCTTCCGCC |
| | AGAGCCCGAG | ACCTTGATCT |
| | A | T |
| HK2 | TGCCACCAGA | CCCGTGCCCA |
| | CTAAACTAGA | CAATGAGAC |
| | CG | |
| PGAM1 | AGGTCACTGC | ACATCACCAC |
| | CTACTGCCTG | GCAGGTTACA |
| | | T |
| LDHA | ATGGCAACTC | CCAACCCCAA |
| | TAAAGGATCA | CAACTGTAAT |
| | GC | CT |
| FOXO1 | GGATGTGCAT | TTTCGGGATT |
| | TCTATGGTGT | GCTTATCTCA |
| | ACC | GAC |

### Virus Production

Pseudotyped HIV_{GKO} and HIV_{NL4-3/Luciferase} viral stocks were generated by co-transfecting (standard calcium phosphate transfection method) HEK293T cells with a plasmid encoding HIV_{GKO} or HIV_{NL4-3/Luciferase}, and a plasmid encoding HIV-1 dual-tropic envelope (pSVIII-92HT593.1) or vesicular stomatitis virus G protein (VSVg), respectively. Medium was changed 6-8 hr post-transfection, and supernatants were collected after 48 hr, centrifuged (20 min, 2000 rpm, RT), filtered through a 0.45 μM membrane to clear cell debris, and then concentrated by ultracentrifugation (22,000 g, 2 hr, 4° C.). Concentrated virions were resuspended in complete media and stored at −80° C. Virus concentration was estimated by p24 titration using the Lenti-X™ p24 Rapid Titer Kit (Clontech).

### Luciferase Reporter Assay

For reactivation of latent HIV-1 provirus, cells were counted and collected as pellets by centrifugation at 1500 rpm for 10 min. Cells were then plated in 96-well U-bottom plates at 1×10⁶ per 200 μl in the presence of 30 μM Raltegravir (Santa Cruz Biotechnology) and the indicated activator. Cells were harvested 48 h after stimulation, washed one time with PBS, and lysed in 60 μl of Passive Lysis Buffer (Promega). After 15 min of lysis, the luciferase activity in cell extracts was quantified with a Perkin Elmer EnSpire 2300 Multimode plate reader after mixing 20 μl of lysate with 100 μl of substrate (Luciferase Assay System-Promega). Relative light units (RLU) were normalized to protein content determined by Bradford assay (BioRad). Data represent average (±SD) of three technical replicates per donor. Cell viability was measured with CellTiter-Blue Cell Viability Assay (Promega).

### T-Cell Activation

CD69 and CD25 expression was measured by flow cytometry gating on CD3⁺CD4⁺ T cells using FITC-labeled antibodies for CD3 (11-0048-42, eBioscience), APC-conjugated CD25 antibodies (17-0259-42, eBioscience), PerCP-labeled antibodies for CD4 (300528, Biolegend), and CD69-V450 (560740, BD Horizon). Staining was performed for 30 min on ice in FACS buffer (PBS, 2% FBS), and samples were analyzed on a BD Biosciences LSRII flow cytometer. Shown are the percentages of positive cells relative to total CD3⁺ CD4⁺ T cells or median fluorescence intensity (MFI).

### Western Blot

Cells were lysed in radioimmunoprecipitation assay buffer (50 mM Tris-HCl, pH 8, 150 mM NaCl, 1% NP-40, 0.5% sodium deoxycholate, and 0.1% SDS, supplemented with protease inhibitor cocktail; Sigma-Aldrich) for 30 min at 4° C. Samples were resuspended in Laemmli buffer for SDS-PAGE. For chemiluminescent detection, enhanced luminol-based chemiluminescent substrate (ECL) and ECL Hyperfilm (Amersham) were used.

### Antibodies

The following primary antibodies were used: SIRT1 (ab104833; Abcam; 8469S; Cell Signaling Technology), SIRT6 (12486; Cell Signaling Technology), SIRT7 (5360; Cell Signaling Technology), β-actin (A5316; Sigma-Aldrich), FoxO1 (2880S; Cell Signaling Technology), Sp1 (sc-14027; Santa Cruz), CD25-APC (17-0259-42; Affymetrix eBioscience), and CD69-APC (310910; BioLegend).

### Cellular Thermal Shift Assay (CETSA)

K562 cells (ATCC) were maintained in appropriate volume of RPMI medium supplemented with 10% fetal bovine serum (Serum Plus—II, Sigma MO, USA), 100 units/mL penicillin and 100 units/mL streptomycin (Corning, VA, USA). Short-term passages (<15) were used for all experiments. For CETSA, K562 cell were freshly seeded the day before the experiment. The day of experiment, equal numbers of cells were counted by Moxi Mini automated cell counter (Orflo) and 0.6×10\hat{ }6 cells per data point were seeded in T-25 cell culture flasks (VWR, PA, USA) in appropriate volume of culture medium. Cells were exposed to 100 nM AS1842856 or equal volume DMSO for 3 hours in an incubator with 5% CO2 and 37° C. Following the incubation, cells were harvested, washed with PBS and diluted in PBS supplemented with EDTA-free complete protease inhibitor cocktail (ROCHE). Then cells were divided into 100 μl aliquots and heated individually at different temperatures for 3 minutes (Thermal cycler, BIO-RAD) followed by cooling for 2 minutes at room temperature. Cell suspensions were freeze-thawed three times using liquid nitrogen. The soluble fraction was separated from the cell debris by centrifugation at 20000×g for 20 minutes at 4° C. The supernatants were transferred to new microcentrifuge tubes and analyzed by SDS-PAGE followed by immunoblotting analysis. CETSA experiments were performed as triplicates in different days. Immunoblotting results were subjected to densitometric analysis (Image J) and melting temperatures of FOXO1, FOXO3 and FOXO4 were determined by Prism 7 (GraphPad) using nonlinear least-squares regression fit to Y=100/(1+10\hat{ }(Log IC50−X)*H), where H=Hill slope (variable). The viability of the cells was assessed in triplicate by trypan blue exclusion.

### Trypan Blue Dye Exclusion

Trypan blue dye exclusion was used to evaluate the cell viability after short term AS1842856 exposure. K562 cells were treated with AS1842856 or DMSO for 0-3 hours and a 10 μL cell aliquot from each sample was briefly mixed with an equal volume of 0.4% (w/v) trypan blue dye solution (Gibco, NY, USA) and counted. Cells with the ability to exclude trypan blue were considered viable.

### Statistical Analysis

Comparisons for two groups were calculated using a paired (comparing populations derived from the same donor) two-tailed Student's t test. Comparisons for more than two groups were calculated using one-way ANOVA, followed by Tukey's multiple comparison tests. Data are presented as mean±SD for technical replicates or mean±SEM for biological replicates. Statistical significance is indicated in all figures by the following annotations: *, P<0.05; **, P<0.01; ***, P<0.001; ****, P<0.0001.

### Chromatin Immunoprecipitation

J-Lat A2 and A72 cells were treated with TNFα (10 ng/ml) for 18 h. Cells were fixed with 1% formaldehyde (v/v) in fixation buffer (1 mM EDTA, 0.5 mM EGTA, 50 mM Hepes, pH 8.0, 100 mM NaCl), and fixation was stopped after 10 min by addition of glycine to 125 mM. The cell membrane was lysed for 15 min on ice (5 mM Pipes, pH 8.0, 85 mM KCl, 0.5% NP40, protease inhibitors). After washing with nuclear swell buffer (25 mM HEPES, pH 7.5, 4 mM KCl, 1 Mm DTT, 0.5% NP-40, 0.5 mM PMSF) and micrococcal nuclease (MNase) digestion buffer (20 mM Tris pH 7.5, 2.5 mM CaCl2), 5 mM NaCl, 1 mM DTT, 0.5% NP-40), the pellet was resuspended in MNase buffer (15 mM Tris-HCl, pH 7.5, 5 mM MgCl2, 1 mM CaCl2), and 25 mM NaCl). Subsequently, samples were incubated with MNase (New England Biolabs) for 10 min at RT. The reaction was quenched with 0.5 M EDTA and incubated on ice for 5 min. Cells were lysed (1% SDS, 10 mM EDTA, 50 mM Tris-HCl, pH 8.1, protease inhibitors), and chromatin DNA was sheared to 200-1000-bp average size through sonication (Ultrasonic Processor CP-130, Cole Parmer). Cellular debris was pelleted, and the supernatant was recovered. Protein A/G Sepharose beads were blocked with single-stranded salmon sperm DNA and BSA, washed and resuspended in immunoprecipitation buffer. Blocked protein A/G Sepharose beads were added to the digested chromatin fractions and rotated at 4° C. for 2 h to preclear chromatin. Lysates were incubated overnight at 4° C. with 5 μg of FOXO1, RelA, histone H4, H4K20me1, H4K20me2, H4K20me3 antibodies, or IgG control. After incubation with protein A/G agarose beads for 2 h and washing three times with low salt buffer (0.1% SDS, 1% Triton X-100, 2 mM EDTA, 20 mM Tris-HCl, pH 8.1, 150 mM NaCl), one time with high salt buffer (0.1% SDS, 1% Triton X-100, 2 mM EDTA, 20 mM Tris-HCl, pH 8.1, 500 mM NaCl) and twice with TE-buffer (1 mM EDTA, 10 mM Tris-HCl, pH 8.1), chromatin was eluted and recovered with Agencourt AMPure XP beads (Beckman Coulter). Bound chromatin and input DNA were treated with RNase H (New England Biolabs) and Proteinase K (Sigma-Aldrich) at 37° C. for 30 min. Immunoprecipitated chromatin was quantified by real-time PCR using the Maxima SYBR Green qPCR Master Mix (Thermo Scientific) and the ABI 7700 Sequence Detection System (Applied Biosystems). The SDS 2.4 software (Applied Biosystems) was used for analysis. The specificity of each PCR reaction was confirmed by melting curve analysis using the Dissociation Curve software (Applied Biosystems). All chromatin immunoprecipitations and qPCRs were repeated at least three times, and representative results were shown.

Primer sequences were:

HIV LTR Nuc1 forward:
5' AGTGTGTGCCCGTCTGTTGT 3′,
HIV LTR Nuc1 reverse:
5′ TTCGCTTTCAGGTCCCTGTT 3′

### Synergy Bliss Model

To analyze combination effects, the Bliss independence model was used. The Bliss score (Δfaxy) was the difference between the calculated reactivation value if the two drugs act independently and the observed combined reactivation values. Synergy was defined as Δfaxy>0, while Δfaxy<0 indicated antagonism. Positive Bliss scores represented dose combinations where the effect was greater than additive.

### Results

### Example 1: FOXO1 is a Specific Regulator of HIV Latency Establishment

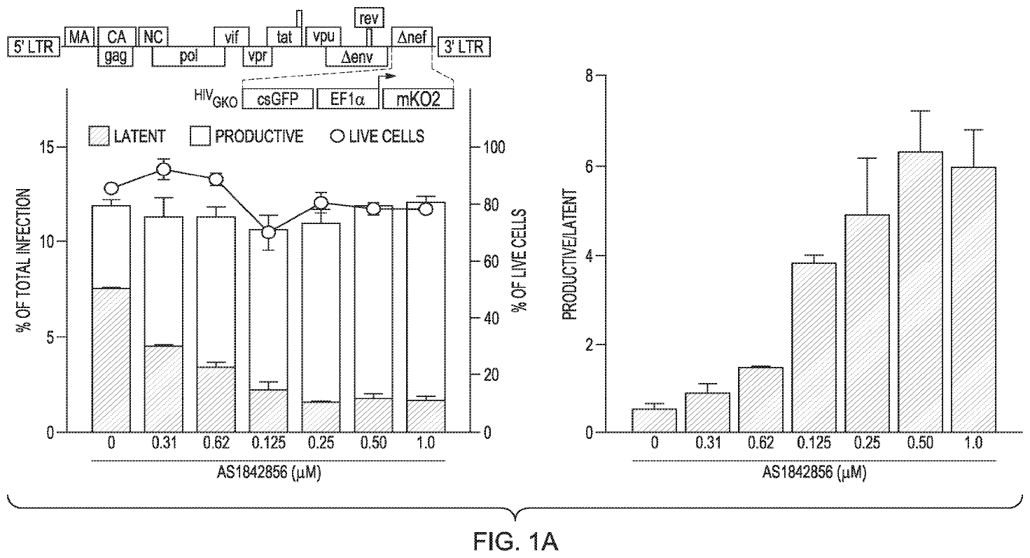

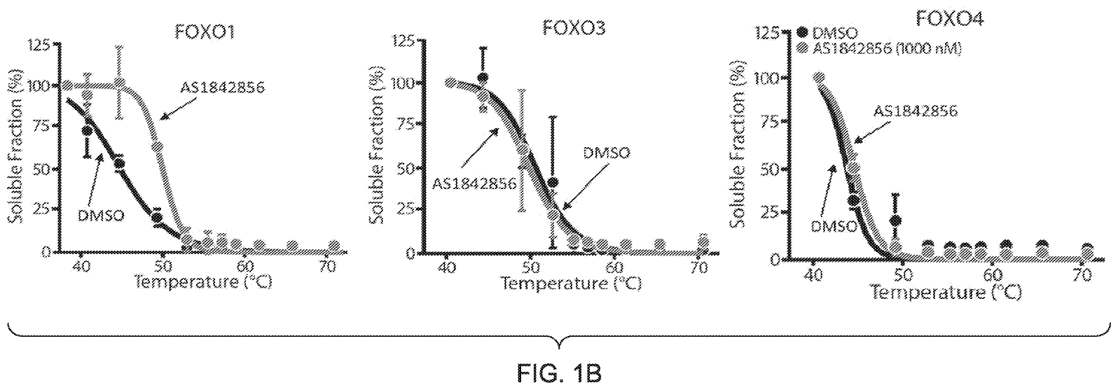

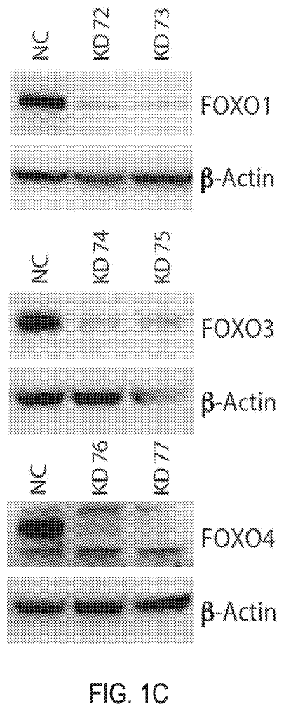

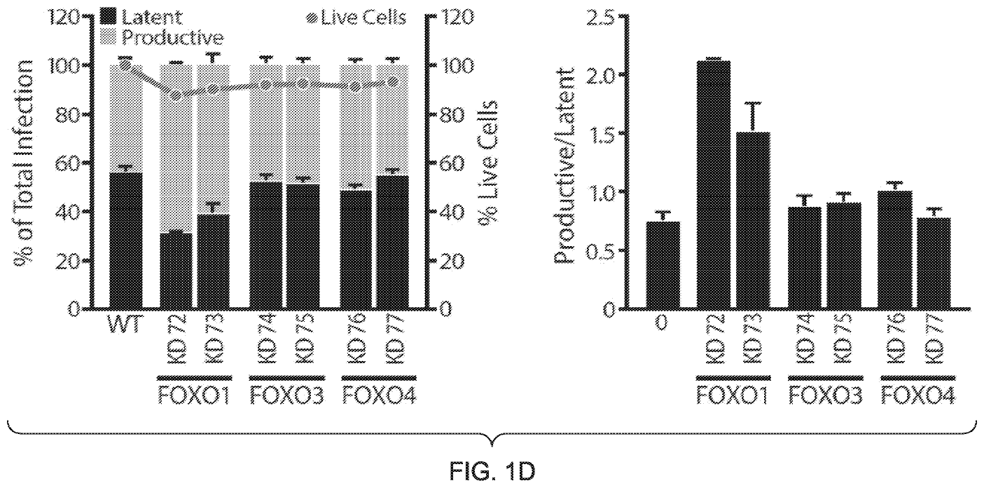

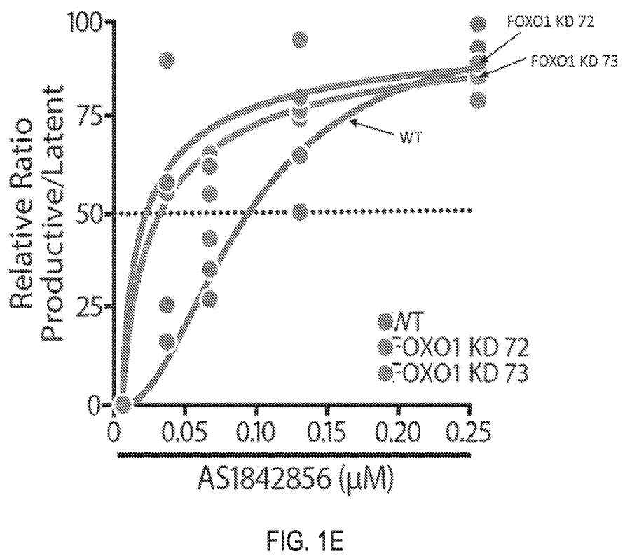

While the effect of FOXO1 inhibitor treatment on productive infection has been tested (Trinite, B. et al. PLoS One 9, (2014)), its effect on latent HIV previously remained unknown. To test the potential of FOXO1 inhibition to affect HIV-1 latency, the new second-generation dual-color reporter virus HIV_{GKO} (LTR-HIV-Δ-env-nefATG-csGFP-EF1α-mKO2) and the FOXO1 inhibitor AS1842856 were employed (FIG. 1, Panel A). In this system, latently-infected K562 cells express the mKO2 fluorophore, while cells containing a productive and active virus express both mKO2 and GFP fluorophores, the latter controlled by the HIV-1 LTR. Upon treatment with increasing concentrations of AS1842856, the proportion of latently infected cells decreased, despite no significant changes in the total infection rate or viability (FIG. 1, Panel A). Concomitantly, the proportion of actively infected cells increased. The IC50 of the inhibitor observed in the described cell culture system was at 60 nM, which is near its in vitro IC50 of 33 nM (Nagashima T. et al. Mol. Pharmacol. 78:961-970. 10.1124/mol.110.065714). Thus, FOXO1 inhibition prevented HIV-1 latency establishment and increased the ratio between actively and latently infected cells.

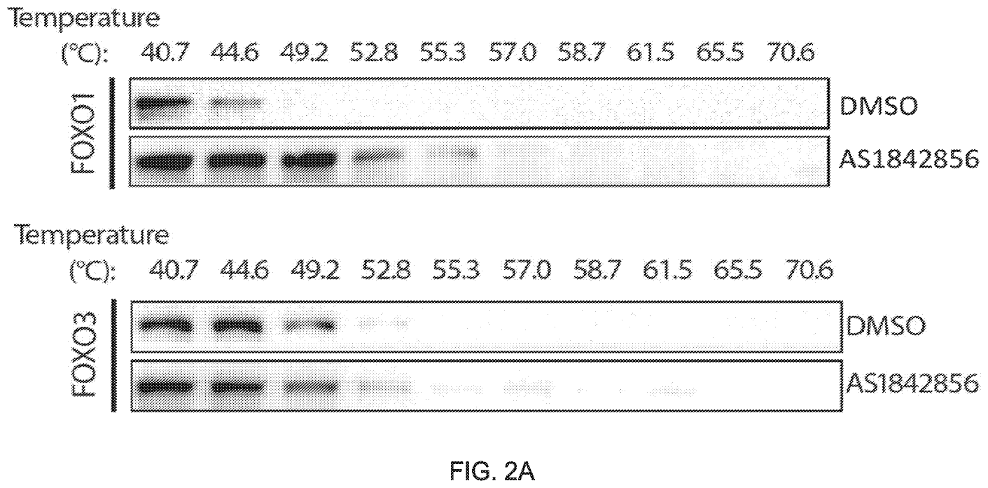

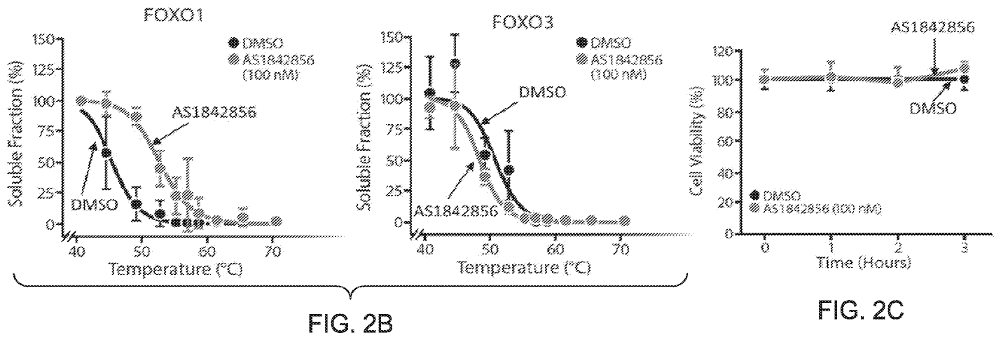

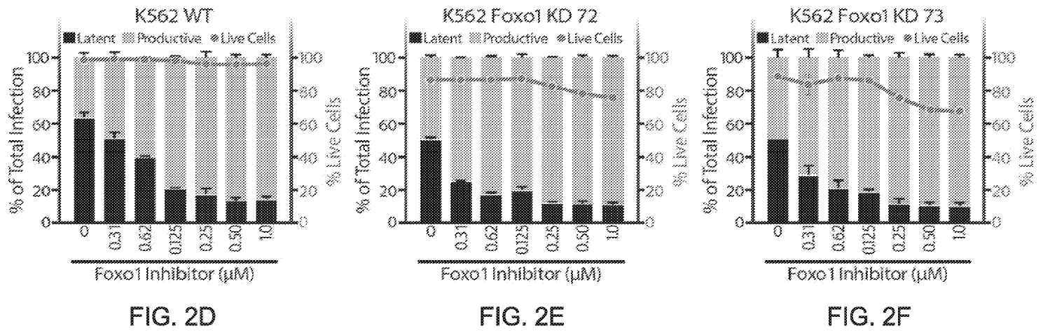

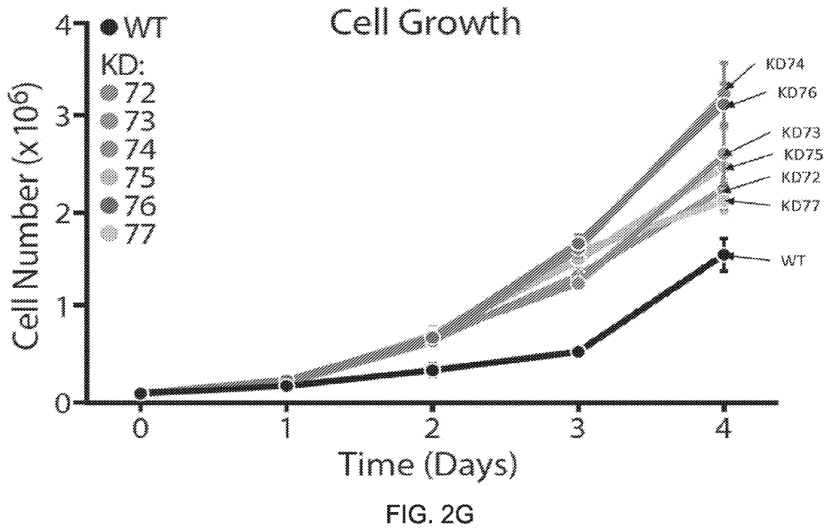

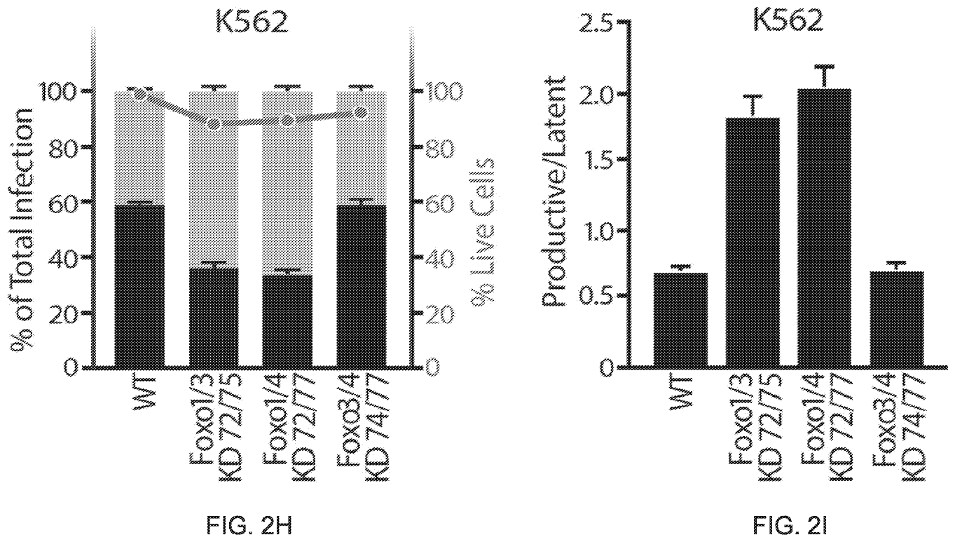

Whether the effect of AS1842856 was mediated by its interaction with FOXO1 was confirmed. To that end, Cellular Thermal Shift Assays (CETSA) were performed against the FOXO isoforms expressed in K562 cells. CETSA is unique in its capability to evaluate biophysical binding under physiological conditions and in living cells. AS1842856-FOXO binding was considered ‘positive’ if there was a significant change in the thermal profile of the protein between the presence and the absence of the drug assessed by western blot (FIG. 2, Panel A). A 7.23° C. shift was observed in FOXO1 melting curve upon AS1842856 treatment (1 μM), whereas no changes were observed in FOXO3 or FOXO4 (FIG. 1, Panel B). Similar results were observed at 100 nM of AS1842856 (FIG. 2, Panels A-C), confirming the specificity of the inhibitor.

To further interrogate the role of other FOXO family members in HIV-1 latency establishment, single knockdowns of FOXO1 (KD 72, 73), FOXO3 (KD 74, 75) and FOXO4 (KD 76, 77) were generated by CRISPR interference (CRISPRi) in K562 cells (FIG. 1, Panel C). Because FOXO proteins control the cell cycle and promote a quiescent state, each knockdown led to an increase in the proliferation rate (FIG. 2, Panel G), in agreement with previous findings on human bladder cancer cells (Shiota, M. et al. Clin. Cancer Res. 16, 5654-5663 (2010)) and MCF-7 breast adenocarcinoma cells (T. Tezil et al, 2012). However, only FOXO1 downregulation increased productive infection and decreased latent infection (FIG. 1, Panel D). To rule out the potential of compensatory effects between isoforms, combinations of double knockdowns were generated. Once again, only in the setting of FOXO1 knockdown was the ratio of productive versus latent infection increased (FIG. 2, Panels H-I). Furthermore, FOXO1 knockdown rendered infected cells more sensitive to AS1842856 treatment, shifting the IC50 (FIG. 1, Panel E and FIG. 2, Panels D-F). Taken together, these data indicate that pharmacological or genetic inhibition of FOXO1 decreases establishment of HIV-1 latency.

FIG. 1, Panels A-E demonstrate that FOXO1 is a specific regulator of HIV latency establishment. FIG. 1, Panel A depicts a schematic representation of HIV_{GKO} dual-labeled HIV-1 reporter. Briefly, K562 cells were treated with increasing concentrations of the FOXO1 inhibitor AS1842856 just after HIV_{GKO} infection. After 3-4 days, the percentage of latent cells and productive cells were quantified by FACS. In left panel, amount of productive or latent cells relative to the total infection rate (bars) and cell viability (dots). In the right panel, ratios of productive versus latent populations upon increasing concentrations of AS1842856 treatments. Data are represented as mean f SD of triplicate values, representative of three independent experiments. FIG. 1, Panel B depicts FOXO1, FOXO3 and FOXO4 CETSA-melting curve shifts upon the presence or absence of AS1842856 1000 nM in K562 cells. Band intensities obtained from western blot analysis were related to the highest western blot signal which has been set to 100%. Relative FOXO-band intensities were plotted against corresponding incubation temperatures and a nonlinear least-squares regression fit was applied. Data represent the mean±SD of three individual experiments. FIG. 1, Panel C depicts the efficiency of FOXO1, FOXO3 and FOXO4 knockdowns with two different sgRNAs checked by western blot. Cells transduced with NC (negative control) sgRNA lentiviruses were used as a control. FIG. 1, Panel D (left) depicts percentage of productive or latent cells relative to the total infection rate and cell viability in the different single knockdown K562 cell lines. FIG. 1, Panel D (right) depicts ratios of productive versus latent populations in the different K562 knockdown cell lines for the different FOXO proteins. Data are represented as mean±SD of triplicate values, representative of two independent experiments. FIG. 1, Panel E depicts ratios of productive versus latent populations upon increasing concentrations of AS1842856 treatments in the WT K562 or in the FoxO1-knockdown cell lines. Data are represented as mean±SD of triplicate values, representative of three independent experiments and fitted into a model.

FIG. 2, Panel A depicts representative western blots of CETSA assays in FOXO1 and FOXO3 in the presence or absence of 100 nM AS1842856. FIG. 2, Panel B depicts FOXO1 and FOXO3 CETSA-melting curves upon the presence or absence of AS1842856 100 nM in K562 cells. Band intensities obtained from western blot analysis were related to the highest western blot signal which has been set to 100%. Relative FOXO-band intensities were plotted against corresponding incubation temperatures and a nonlinear least-squares regression fit was applied. Data represent the mean±SD of three individual experiments. FIG. 2, Panel C depicts cell viability of CETSA assay in FOXO1 experiments assessed by Trypan Blue exclusion. FIG. 2, Panels D-F depict percentage of productive or latent cells relative to the total infection rate and cell viability upon increasing concentrations of AS1842856 treatment in the WT K562 (FIG. 2, Panel D) or in the FOXO1-knockdown cell lines KD-72 (FIG. 2, Panel E) and KD-73 (FIG. 2, Panel F). Data are represented as mean±SD of triplicate values. FIG. 2, Panel G depicts cell growth analysis of WT, FOXO1, FOXO3 and FOXO4 single knockdowns K562 cell lines. FIG. 2, Panel H depicts percentage of productive or latent cells relative to the total infection rate and cell viability in double knockdowns K562 cell lines. FIG. 2, Panel I, depicts ratios of productive versus latent populations upon increasing concentrations of AS1842856 treatments in double knockdowns K562 cell lines. Data are represented as mean±SD of two independent experiments.

### Example 2: FOXO1 Inhibition Reactivates HIV-1 from Latency

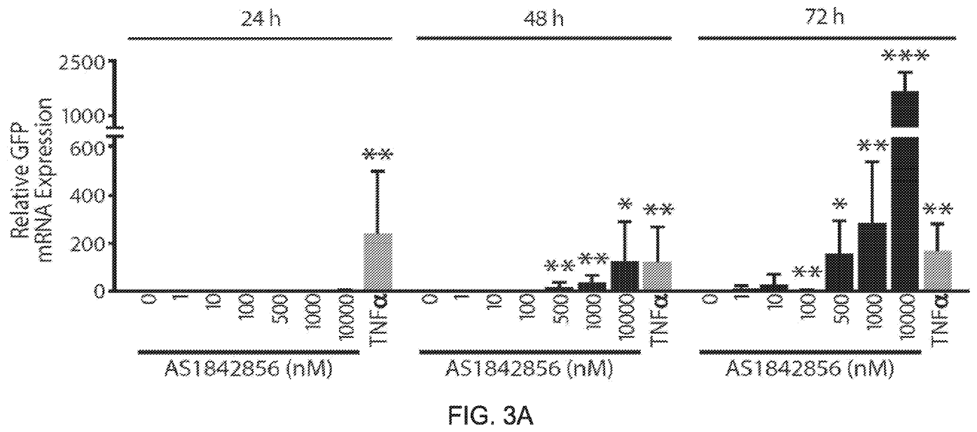

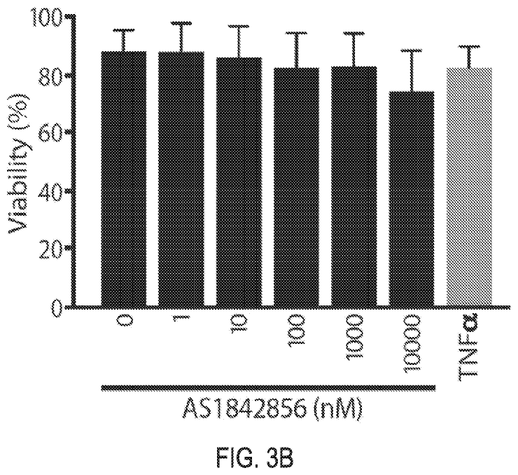

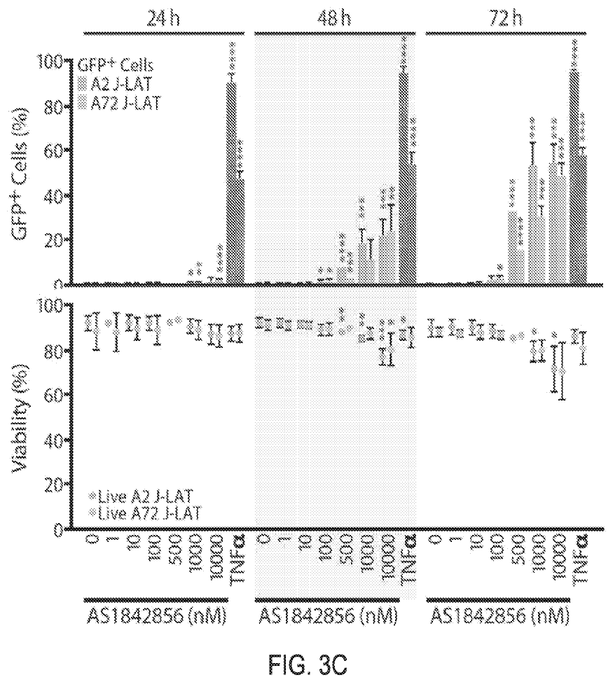

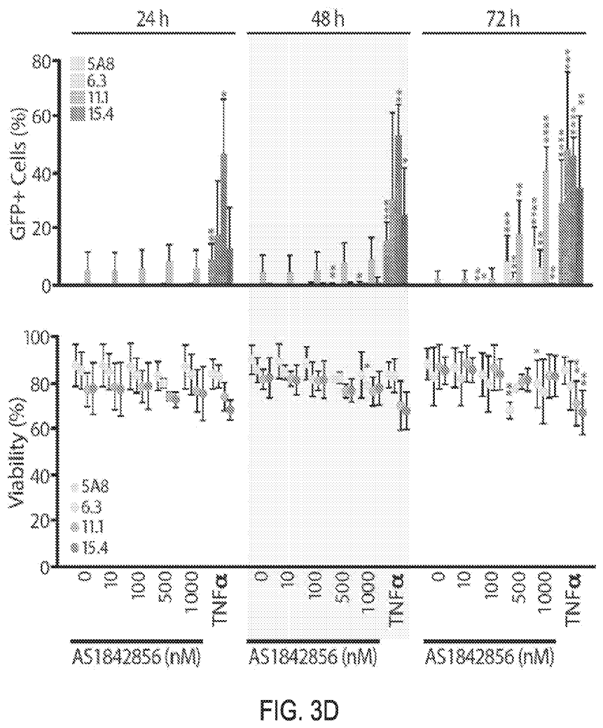

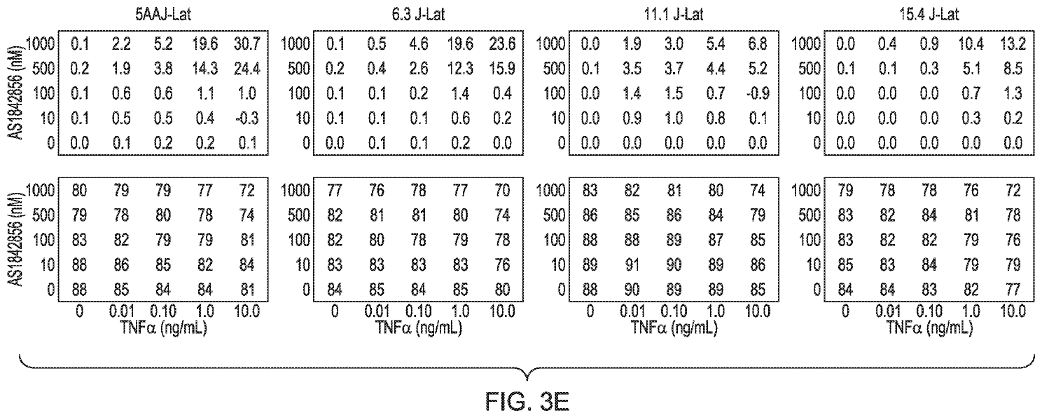

The J-Lat clonal cell lines are Jurkat cells that contain a stably integrated, latent HIV-1 (HXB) Δenv provirus with a GFP reporter gene in place of the nef gene (Jordan, A. et al. EMBO J. 22, 1868-1877 (2003)). In this system, reactivation from established latency can be monitored through GFP expression. After 48 and 72 hours of treatment, AS1842856 stimulated GFP mRNA expression in J-Lat 5A8 cells to a similar degree as TNFα, a known latency reversing agent (LRA) in J-Lat cells, indicating that FOXO1 inhibition can also reverse already established latency (FIG. 3, Panel A). No major effects on cell viability were observed (FIG. 3, Panel B). Notably, maximal activation by TNFα was seen after 24 hours while AS1842856-induced activation progressively increased only after this time point.

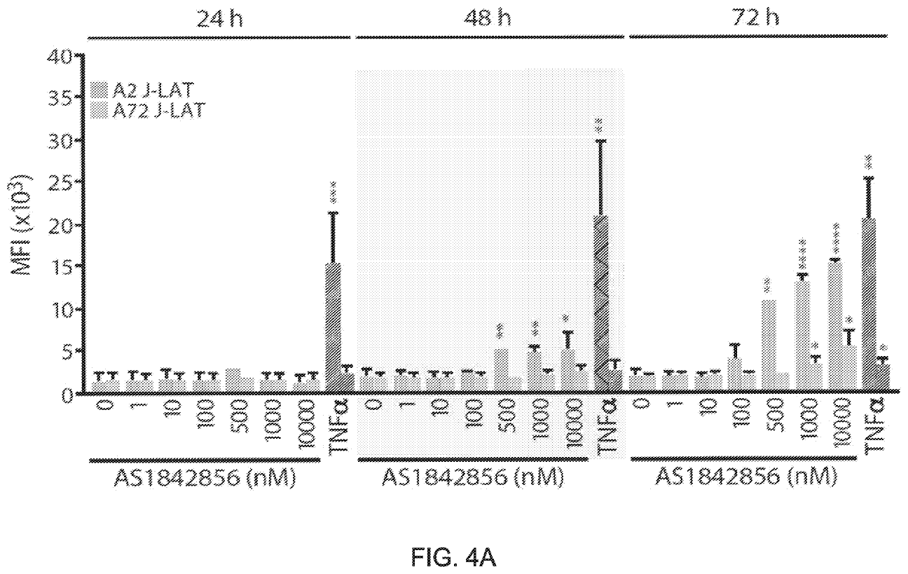

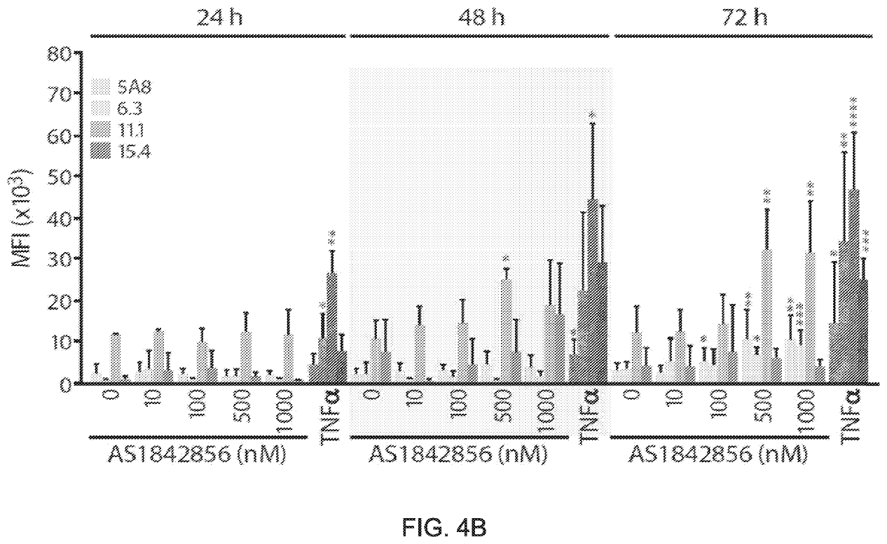

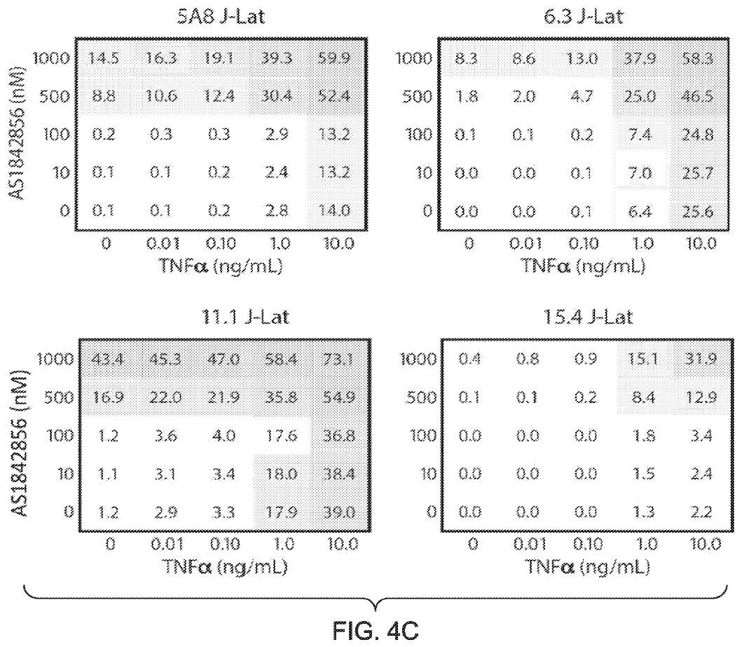

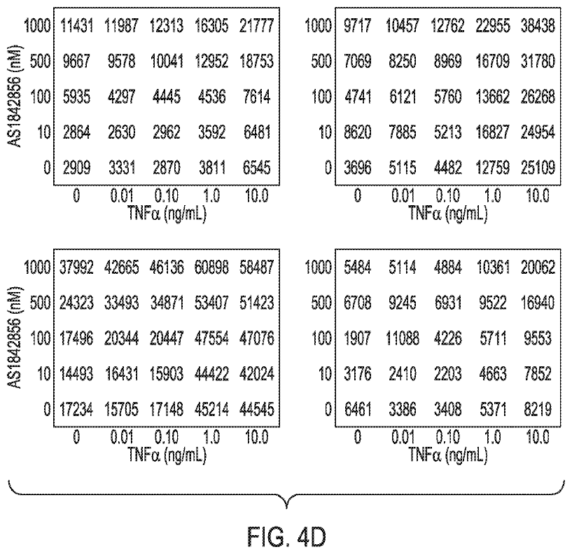

Treatment with AS1842856 also increased HIV-driven GFP expression in a dose-dependent manner at the protein level, as determined by flow cytometry (FIG. 3, Panels C, D). Because previous work found that FOXO1 and FOXO4 antagonize Tat-mediated transactivation of the HIV-1 promoter through the repression of the viral transactivator Tat (Oteiza et. al. J. Gen. Virol. 98, 1864-78 (2017)), the ability of AS1842856 to reverse HIV latency in J-Lat cells with and without Tat was tested. AS1842856 increased GFP expression in both J-Lat A2 cells, which do contain Tat, and in A72 cells, which do not (Jordan, A. et al. EMBO J. 22, 1868-1877 (2003)), indicating that the effect of FOXO1 inhibition on HIV reactivation is Tat-independent (FIG. 3, Panel C and FIG. 4, Panel A). Again, no effect on cell viability was observed.

To rule out effects based on clonal variation or proviral integration sites, AS1842856-induced HIV reactivation in four J-Lat clonal cell lines was compared with the latent HIV-1 inserted at distinct genomic sites. After 72 hours of treatment, AS1842856 reactivated HIV in each cell line, as determined by percentage of GFP+ cells (FIG. 3, Panel D) and the median fluorescence intensity (FIG. 4, Panel B). In all cell lines, viability was unaffected, and treatment with TNFα induced GFP expression as early as 24 hours after treatment as expected (FIG. 3, Panel D). Thus, the effect of AS1842856 is independent from the proviral integration site.

Given the different time courses of TNFα- and AS1842856-induced HIV reactivation, both LRAs may target different mechanisms of reactivation and might synergize. To test this hypothesis, multiple J-Lat lines were co-treated with increasing concentrations of AS1842856 and TNFα (FIG. 4, Panels C, D). In the Bliss independence model, positive Bliss scores indicate dose combinations that result in greater than additive effects. Greater than additive effects between both compounds across different doses in all clones was observed (FIG. 3, Panel E). Not surprisingly, the BLISS score was lowest in J-Lat 11.1 cells, which responded best to AS1842856 mono-treatment. Again, major changes in viability in co-treated cell lines were not observed (FIG. 3, Panel E). Collectively, this data demonstrated the reversal of established latency by FOXO1 inhibition and indicated a molecular mechanism of activation different from TNFα.

FIG. 3, Panels A-E, demonstrate that FOXO1 inhibition reactivates HIV1 from latency. FIG. 3, Panel A, depicts results of treatment of a J-Lat cell line 5A8 with increasing concentrations (1-10000 nM) of AS1842856 for 24, 48 and 72 h. HIV-GFP mRNA reactivation was assessed by RT-qPCR and normalized to RPL13A mRNA. Data represent average SD of three independent experiments. p-value relative to the control at each time point. FIG. 3, Panel B, depicts cell viability assessed by flow cytometry at 72 h of the same experiment as in A. Cell viability was measured by gating on both the live population at the forward scatter (FSC) and side scatter (SSC) plot and then the viable cells after staining with the viability dye. FIG. 3, Panel C, depicts results of treatment of J-Lat cell lines A2 and A72 with increasing concentrations of AS1842856 for 24, 48 and 72 h. HIV-GFP reactivation (bars) and cell viability (dots) were analyzed by FACS. HIV-GFP reactivation is reported as a percentage of GFP-expressing cells (% GFP+ cells). Data represent average f SD of at least three independent experiments. FIG. 3, Panel D, depicts the same experiments as in FIG. 3, Panel C, but performed in different J-Lat cell lines 5A8, 6.3, 11.1 and 15.4. Data represent average f SD of at least three independent experiments. 10 ng/mL TNFα was used as control. FIG. 3 Panel E depicts results of treatment of J-Lat cell lines 5A8, 6.3, 11.1 and 15.4 for 72 h with increasing concentrations of both AS1842856 (Y-axis) and TNFα (X-axis) alone or in combination and analyzed by FACS. Calculation of synergy for drug combinations using the Bliss independence model applied to the HIV-GFP reactivation measured as a percentage of GFP-expressing cells (% GFP+ cells). Synergy (top tables) and cell viability (bottom tables) data points represent the mean effect of three independent replicates. p-value: *p<0.05, **p<0.01, ***p<0.001, ****p<0.0001.

FIG. 4, Panels A-D depict results of J-Lat cell lines treated with different concentrations of AS1842856. FIG. 4, Panel A, depicts results of treatment of J-Lat cell lines A2 and A72 with increasing concentrations of AS1842856 for 24, 48 and 72 h. HIV-GFP reactivation was analyzed by flow cytometry. HIV-GFP reactivation is reported as a Mean Intensity Fluorescence (MFI) of GFP-expressing cells. Data represent mean±SD of at least three independent experiments. FIG. 4, Panel B, depicts the same experiments as in FIG. 4, Panel A, but performed in different J-Lat cell lines 5A8, 6.3, 11.1 and 15.4. Data represent average SD of at least three independent experiments. 10 ng/mL TNFα was used as control. FIG. 4, Panel C, depicts results of treatment of J-Lat cell lines 5A8, 6.3, 11.1 and 15.4 for 72 h with increasing concentrations of both AS1842856 (Y-axis) and TNFα (X-axis) alone or in combination and analyzed by FACS. HIV-GFP reactivation is reported as a percentage of GFP-expressing cells (% GFP+ cells) or FIG. 4, Panel D, depicts Mean Intensity Fluorescence (MFI). Data represent average SD of three independent experiments. p-value: *p<0.05, **p<0.01, ***p<0.001, ****p<0.0001.

### Example 3: FOXO1 Inhibition Prevents Latency Establishment and Reactivates HIV-1 Transcription in Primary CD4⁺ T Cells

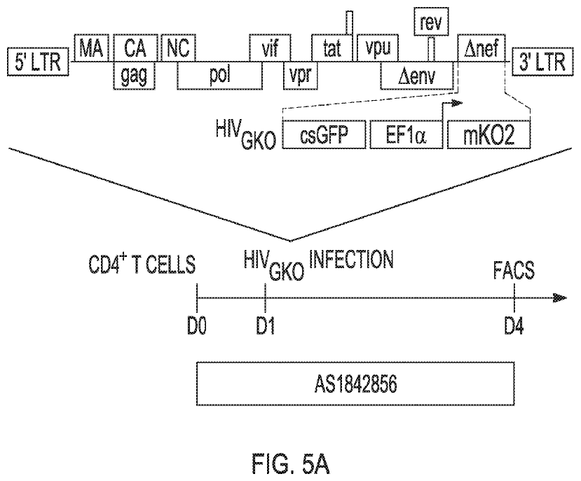

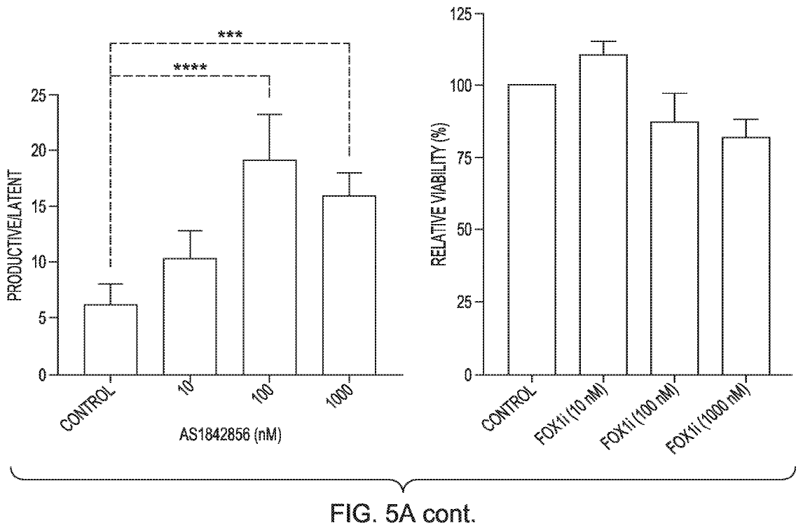

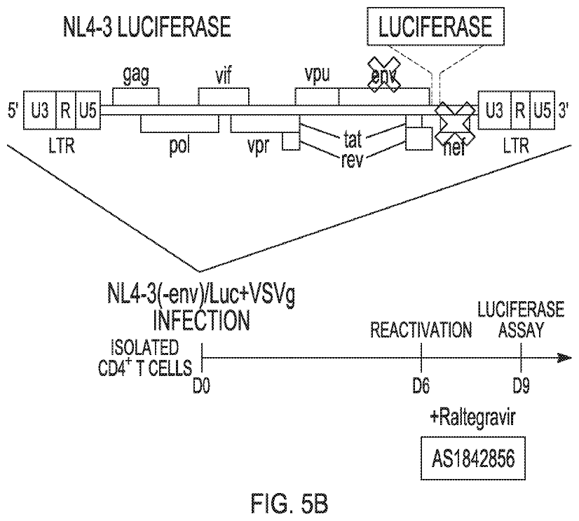

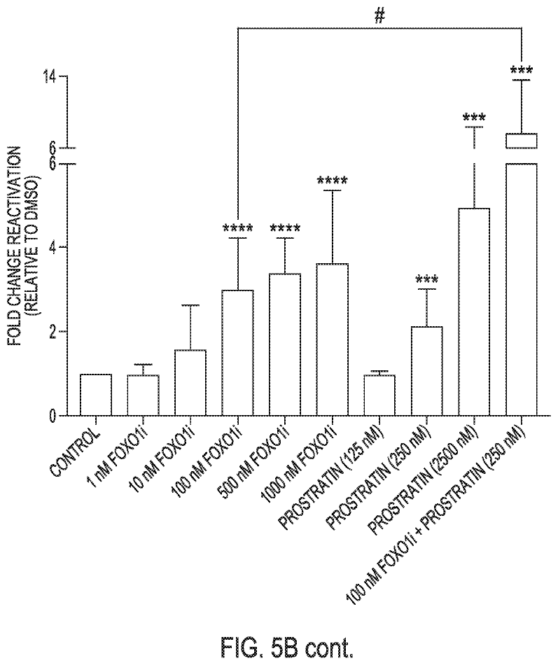

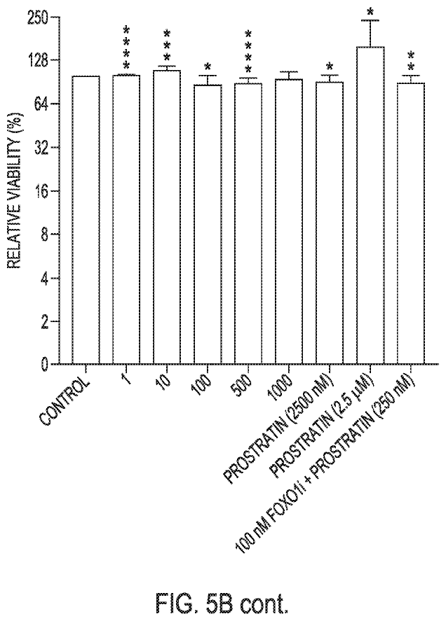

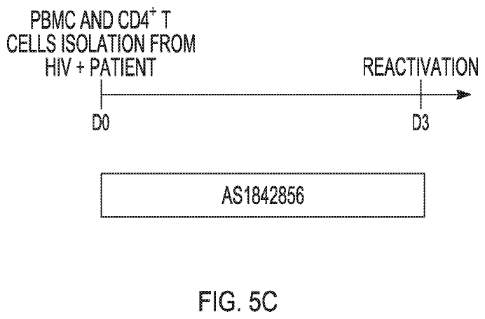

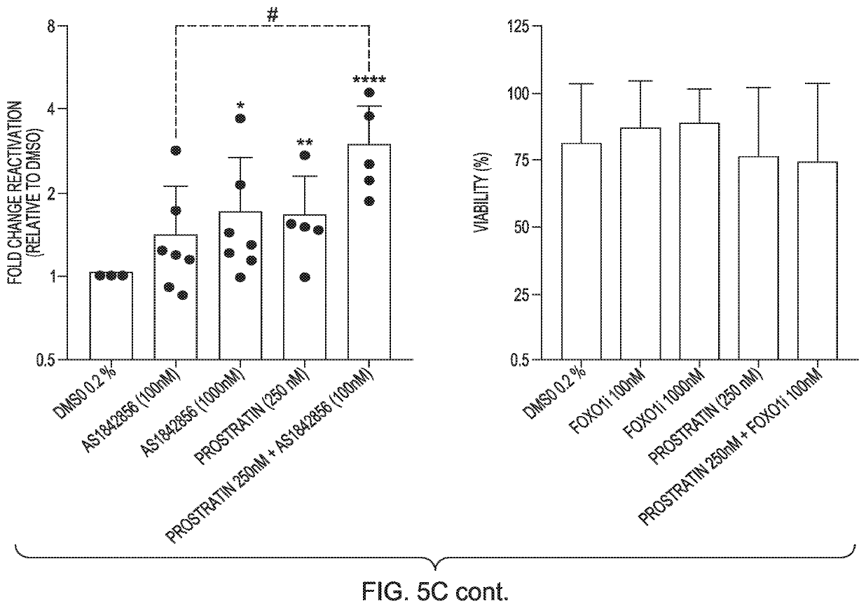

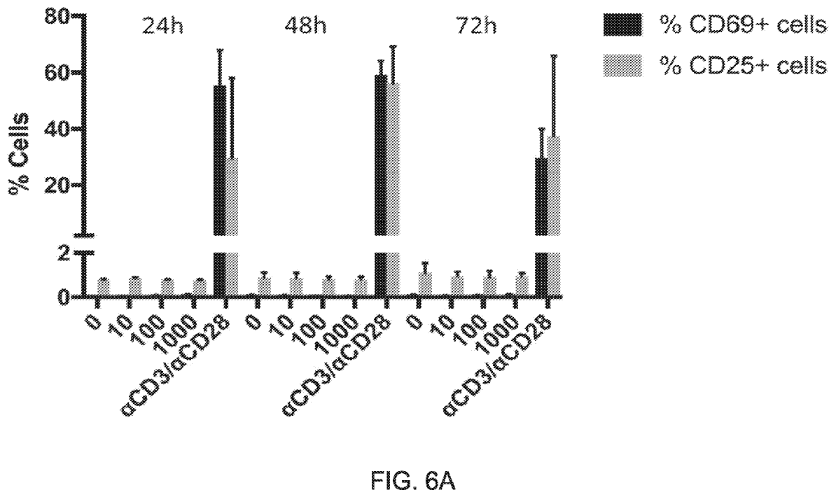

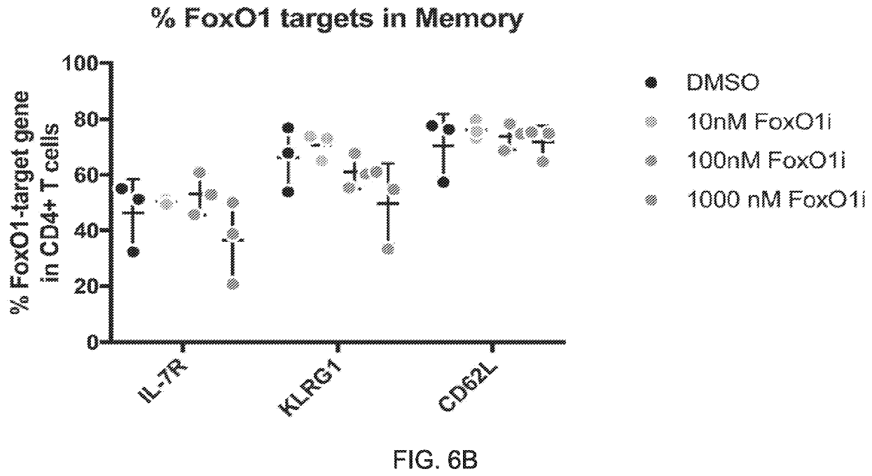

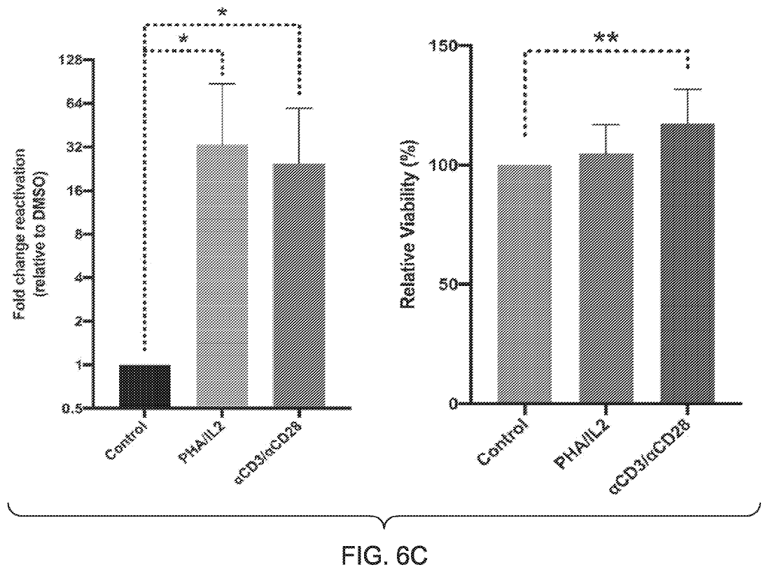

In order to verify these results in primary T-cell models of HIV latency, the capacity of FOXO1 inhibition to impair HIV latency establishment was measured by infecting resting CD4⁺ T cells isolated from anonymous blood donors with HIV_{GKO} while treating the cells with increasing doses of AS1842856 for 72 hours. As described above for K562 cells, the inhibition of FOXO1 promoted productive infection and decreased the establishment of latency (FIG. 5, Panel A). The ability of AS1842856 to reverse established HIV latency in primary cells was also tested by spin-infecting resting CD4⁺ T cells with a pseudotyped HIV reporter virus containing the firefly luciferase gene (Lassen, K. et al. PLoS One 7, (2012)). After 6 days in culture, cells reactivated luciferase expression in response to increasing concentrations of FOXO1 inhibitor (AS1842856) (FIG. 5, Panel B) as well as in response to generalized T cell activation induced with either a combination of phytohemagglutinin and interleukin-2 (PHA/IL-2) or with antibodies directed against the CD3 and CD28 receptors (FIG. 6, Panel A). Although T cell activation reactivated HIV more efficiently, it also caused signs of generalized T cell activation as measured by the expression of the CD69 and CD25 activation markers on the cell surface, which was not observed after FOXO1 inhibition (FIG. 6, Panel B). A combination of AS1842856 (100 nM) with a low dose of prostratin (250 nM), a natural protein kinase C activator inducing partial T-cell activation, significantly increased the AS1842856 effect in primary T cells, mirroring the synergistic effect of TNFα in J-Lat cells. No changes in primary T-cell viability was observed in response to FOXO1 inhibition (FIG. 5, Panels A-C).

The same was observed in CD4⁺ T cells, isolated from five HIV-infected patients on antiretroviral therapy for at least 6 months and with undetectable viral loads (<50 copies/ml). Cells were treated ex vivo with two concentrations of AS1842856 (100 and 1000 nM), prostratin (250 nM) or combinations thereof for 3 days, and HIV RNA induction was measured by digital droplet PCR (ddPCR). A two-fold induction was observed with AS1842856 treatment alone, which was significantly enhanced with prostratin cotreatment to similar levels as PHA/IL-2 treatment (FIG. 5, Panel C and FIG. 6, Panel C). These results confirm a role of FOXO1 in the establishment and maintenance of HIV latency in primary resting T cells.

FIG. 5, Panels A-C demonstrate that FOXO1 inhibition prevents latency establishment and reactivates HIV in primary CD4⁺ T cells and HIV-infected CD4⁺ T cells. FIG. 5, Panel A, depicts a schematic representation of HIV_{GKO} dual-labeled HIV-1 reporter and strategy used to treat primary CD4⁺ T cells purified from blood of healthy donors and pre-treated for 24 h with increasing concentrations of AS1842856. After infection with HIV_{GKO}, resting cells were treated for 72 h with the same amounts of increasing concentrations of AS1842856 as in the pre-treatment. Ratios of productive versus latent populations of infected CD4⁺ T cells from four different healthy donors after treatment with increasing concentrations of AS1842856 (right side—left panel). Histogram plot of percent live cells for each drug treatment relative to the control are shown (right side —right panel). Data are represented by mean±SD of four different donors. FIG. 5, Panel B, depicts a schematic representation of HIV_{NL4-3 Luciferase} reporter virus and an experimental procedure and results with primary CD4*T cells. Briefly, CD4⁺ T cells purified from blood of healthy donors were infected with HIV_{NL4-3 Luciferase VSVg-pseudotyped} and, after 6 days, the virus was reactivated for 72 h with increasing concentrations of AS1842856 and prostratin. HIV reactivation was measured by luciferase activity and cell viability by flow cytometry. Data are mean±SEM of 3/5-13 individual donors. FIG. 5, Panel C, demonstrates that AS1842856/prostratin-treatment of CD4⁺ T cells from HIV-infected patients on antiretroviral-therapy with undetectable viral load led to an increase in fold change of cell-associated HIV mRNA measured by ddPCR. p-value: *p<0.05, **p<0.01, ***p<0.001, ****p<0.0001, respect to Control/DMSO; #p<0.05, respect to AS1842856 (100 nM).

FIG. 6, Panels A-D demonstrate that FoxO1 inhibition prevents latency establishment and reactivates HIV in primary CD4+ T cells. FIG. 6, Panel A, the cell surface CD69 and CD25 T cell activation markers were measured by FACS in CD4⁺ T cells upon AS1842856 treatment for 24, 48 and 72 h. 10 μg/mLαCD3 and 1 μg/mL αCD28 was used as control. Data is shown as percentage of positive cells and as mean±SD of two biological replicates. FIG. 6, Panel B, depicts the same as in FIG. 6, Panel A, but measuring FOXO1 target genes (IL7R, KLRG1, CD62L) after 48 h of AS1842856 treatment (n=3). FIG. 6, Panel C, depicts HIV reactivation measured by luciferase activity and cell viability measured by flow cytometry assessed in CD4⁺ T cells purified from blood of healthy donors and infected with HIV_{NL4-3 Luciferase VSVg-pseudotyped}. Cells were allowed to rest for 6 days before 72 h reactivation was induced with PHA/IL-2 and 10 μg/mL αCD3 and 1 μg/mLαCD28. Data are mean±SEM of at least 3 individual donors. Data represent average SD of 9-13 independent experiments. FIG. 6, Panel D, depicts fold change of cell-associated HIV mRNA measured by ddPCR of CD4⁺ T cells of nine HIV-infected patients on antiretroviral-therapy with undetectable viral load treated with PMA and Ionomycin. Cell viability was assessed by Trypan Blue exclusion. Data are represented as mean±SD of nine independent experiments. p-value: *p<0.05, **p<0.01, ***p<0.001, ****p<0.0001.

### Example 4: Transcriptional Reprogramming in Response to FOXO1 Inhibition in Primary CD4⁺ T Cells

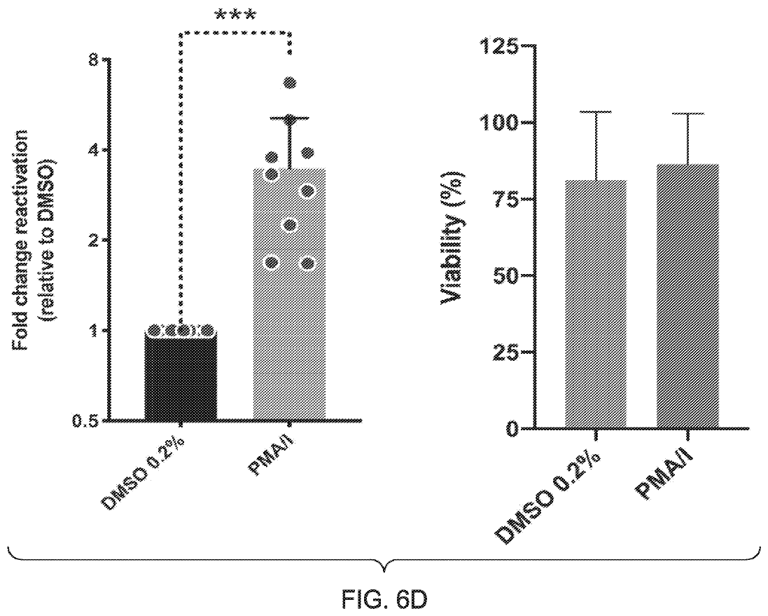

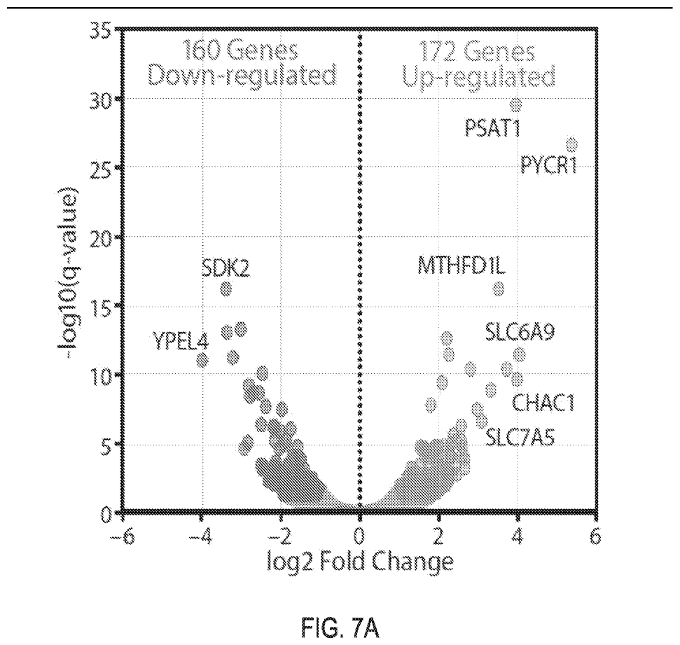

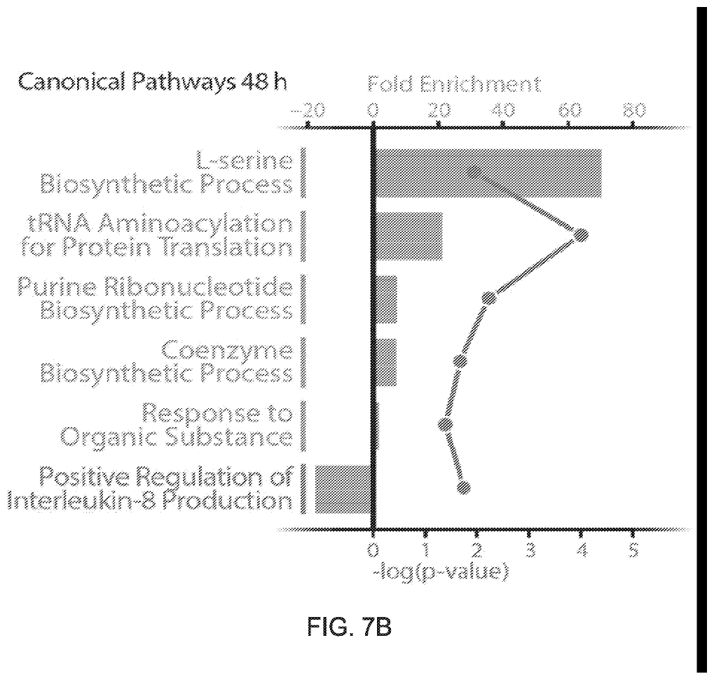

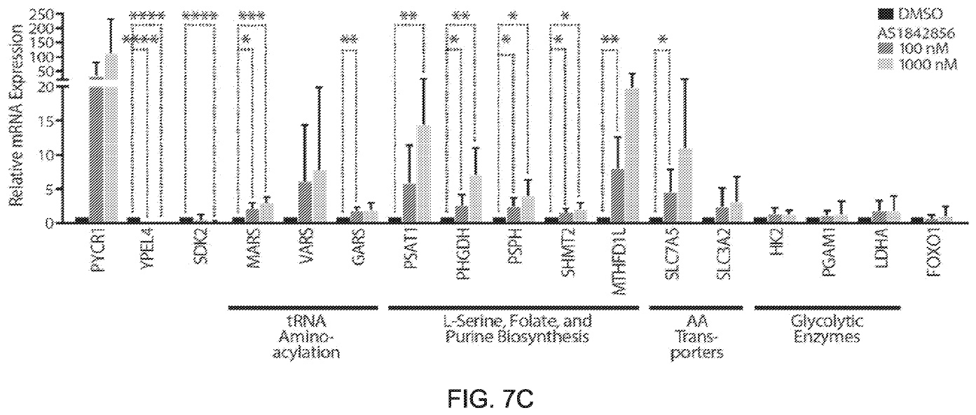

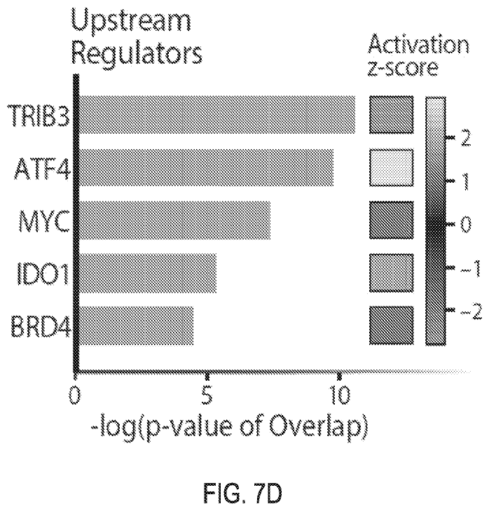

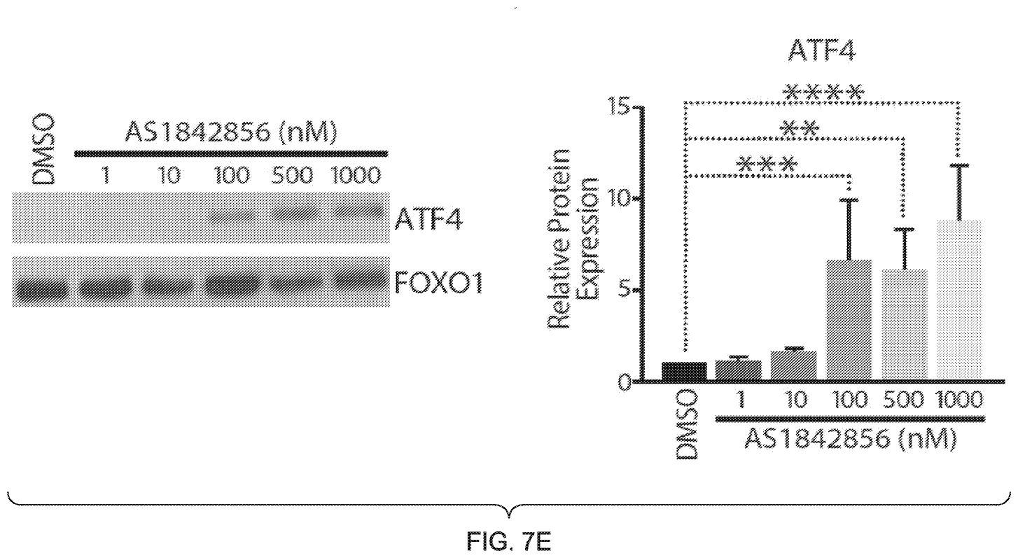

To determine how FOXO1 inhibition changes transcription of resting CD4⁺ T cells, RNA-Sequencing (RNA-Seq) of primary CD4⁺ T cells treated with AS1842856 or DMSO for 48 hours was performed, the first time point at which HIV-1 reactivation was observed. 172 genes were significantly up-regulated and 160 genes were significantly down-regulated by AS1842856 (q<0.05) (FIG. 7, Panel A). When analyzed by Gene Ontology Enrichment analysis to integrate the differentially expressed transcripts into biological pathways, significant enrichment was observed for genes involved in the biosynthesis of L-Serine (e.g. PSAT1, PHGDH), tRNA charging (e.g. MARS, VARS, GARS), biosynthesis of purine (e.g. PFAS, PAICS) and coenzyme and folate metabolism (e.g. MTHFD1L, SHMT1) in genes upregulated after FOXO1 inhibition. Down-regulated genes included those involved in IL-8 production (e.g. endosomal Toll-like receptors 3, 7 and 8) (FIG. 7, Panel B).

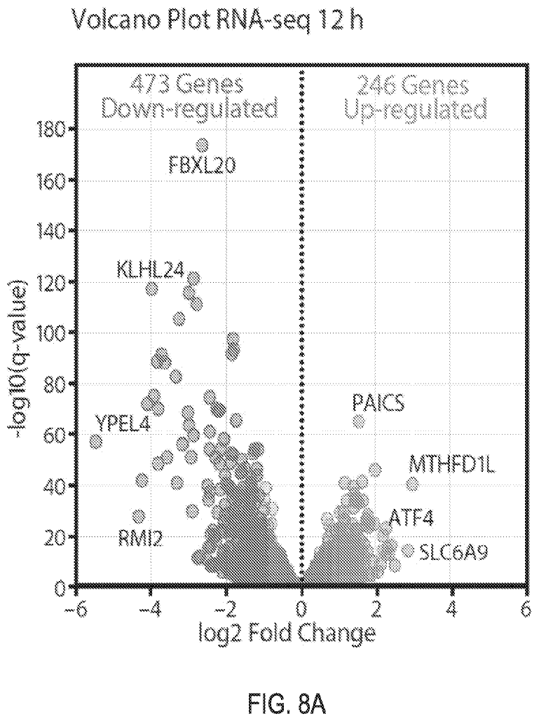

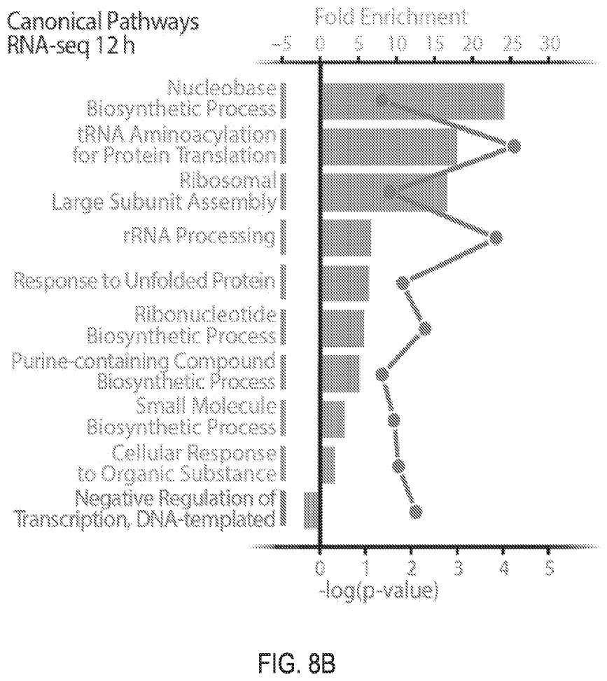

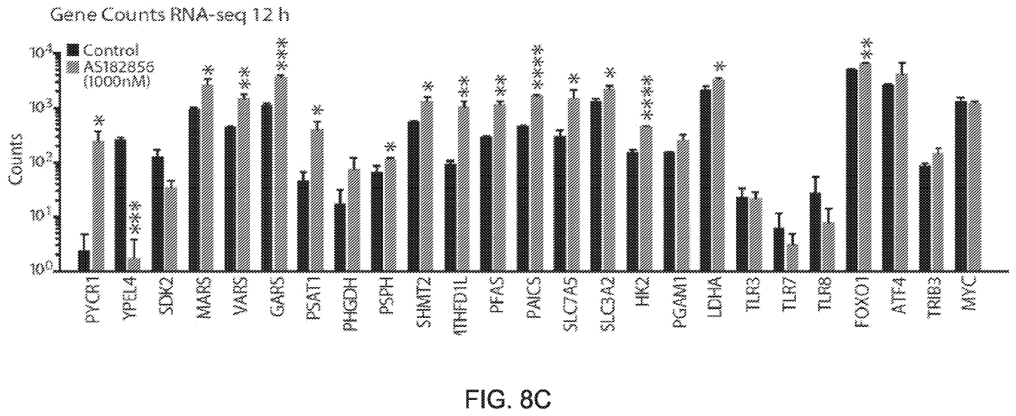

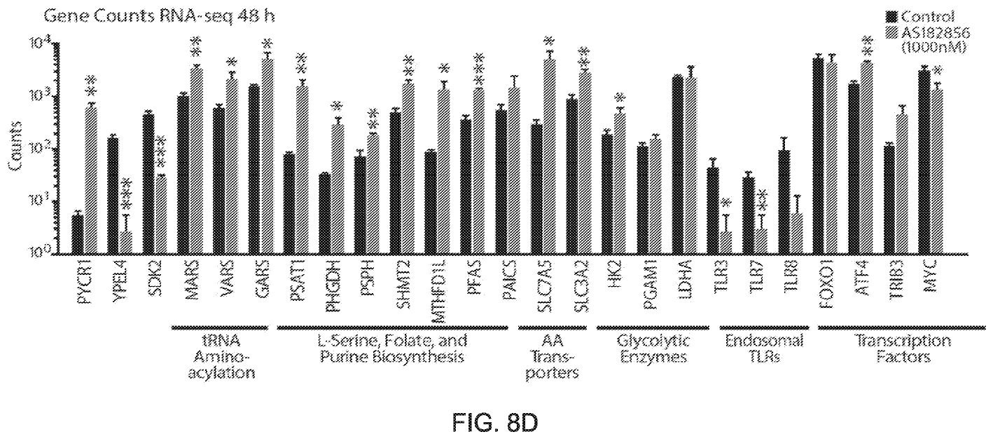

The RNA-Seq results were validated by measuring read counts of the selected genes representative of different pathways (FIG. 8, Panel D) as well as by Real-Time Quantitative PCR (RT-qPCR) (FIG. 7, Panel C). Although FOXO1 has been previously connected with the prevention of glycolysis (Nakae J. et al. (2002) Nat Genet 32:245-253.), treatment with AS1842856 did not affect expression of glycolytic enzymes or FOXO1 itself. Upstream regulator analysis identified TRIB3 and ATF4 as the top regulators of AS1842856-induced gene changes (FIG. 7, Panel D). Specifically, the algorithm predicted that TRIB3 was highly inhibited, while ATF4 was activated (FIG. 7, Panel D). TRIB3 is known to inhibit ATF4 (Ohoka, N. et al. EMBO J. 24, 1243-1255 (2005), Deval, C. et al. J. Biol. Chem. (2007). doi:10.1074/jbc.M611723200), a transcription factor specifically upregulated at the protein level as part of the integrated stress response (ISR) inducing phosphorylation of the translation initiation factor eIF2α by several upstream ER-resident kinases when global protein translation is shut down. RNA-Seq analysis of AS1842856-treated CD4⁺ T cells at 12 hours after treatment showed upregulation of pathways including ribosomal large subunit assembly, rRNA processing, and the unfolded protein response, supporting a role of ATF4 and ISR in gene changes after FOXO1 inhibition (FIG. 8, Panel A). In line with this hypothesis, FOXO1 inhibition increased expression of ATF4 protein levels in CD4⁺ T cells across multiple donors (FIG. 7, Panel E).

FIG. 7, Panels A-E, depict marked upregulation of the regulator ATF4 in response to FOXO1 inhibition in primary CD4⁺ T cells. FIG. 7, Panel A depicts a volcano plot from the RNA-Seq data comparing CD4⁺ T cells treated with 1000 nM AS1842856 versus DMSO control for 48 h. Up-regulated (blue—right) or down-regulated (red —left) genes are q-value<0.05 and log 2 fold change≥1 or ≤−1, respectively. FIG. 7, Panel B, depicts analysis of the most dysregulated canonical pathways for the up- and down-regulated genes by Ingenuity Pathway Analysis. FIG. 7, Panel C, depicts confirmation of up- or down-regulated expression of specific genes from different altered pathways by mean read counts from sequencing. FIG. 7, Panel D, depicts analysis of the five top upstream regulators and table with their activation z-scores according to the RNA-Seq data. FIG. 7, Panel E depicts a representative blot of protein expression of dysregulated genes of interest (left) and densitometry analysis (right) was performed from three individual donors. Data are mean±SD. p-value: *p<0.05, **p<0.01, ***p<0.001, ****p<0.0001.

FIG. 8, Panels A-D depict read counts of the selected genes representative of different pathways as well as Real-Time Quantitative PCR (RT-qPCR) results. FIG. 8, Panel A, depicts a volcano plot from the RNA-Seq data comparing CD4⁺ T cells treated with 1000 nM AS1842856 versus DMSO control for 12 h. Up-regulated (blue—right) or down-regulated (red—left) genes are q-value<0.05 and log 2 fold change≥1 or ≤−1, respectively. FIG. 8, Panel B depicts analysis of the most dysregulated canonical pathways for the up- and down-regulated genes at 12 h by Ingenuity Pathway Analysis. FIG. 8, Panels C-D, depict confirmation of up- or down-regulated expression of specific genes from different altered pathways by mean counts from sequencing at 12 h (FIG. 8, Panel C) and 48 h (FIG. 8, Panel D). Data are represented as mean±SD of three independent experiments. p-value: *p<0.05, **p<0.01, ***p<0.001, ****p<0.0001.

### Example 5: FOXO1 Inhibition Induces HIV-1 Reactivation Via ATF4 and NFAT

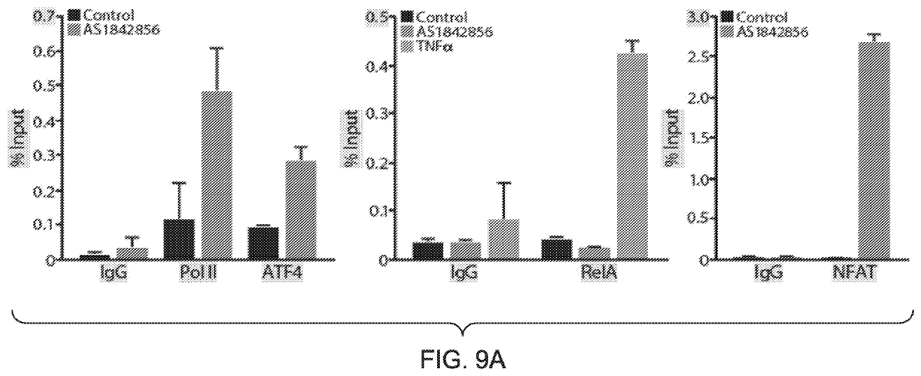

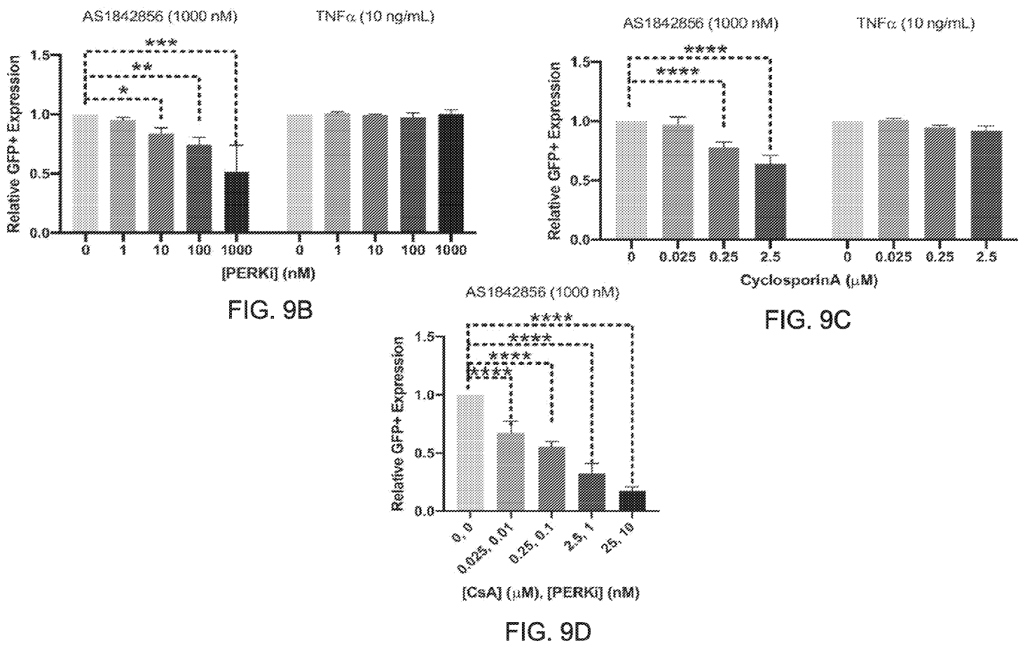

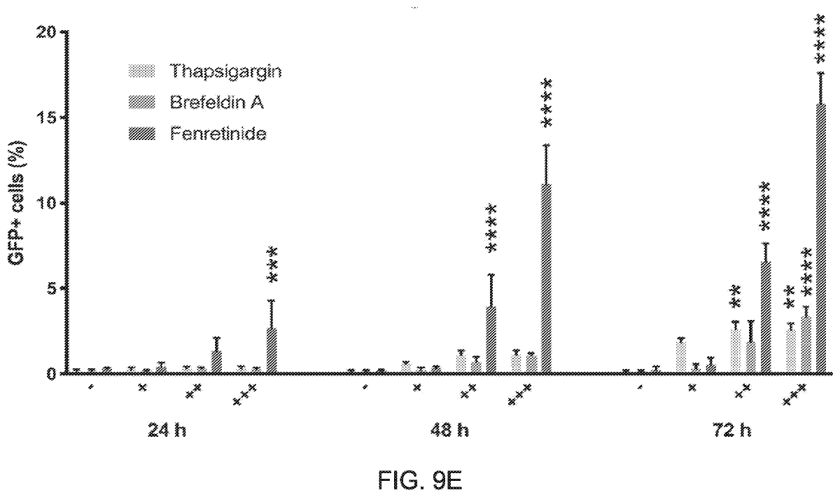

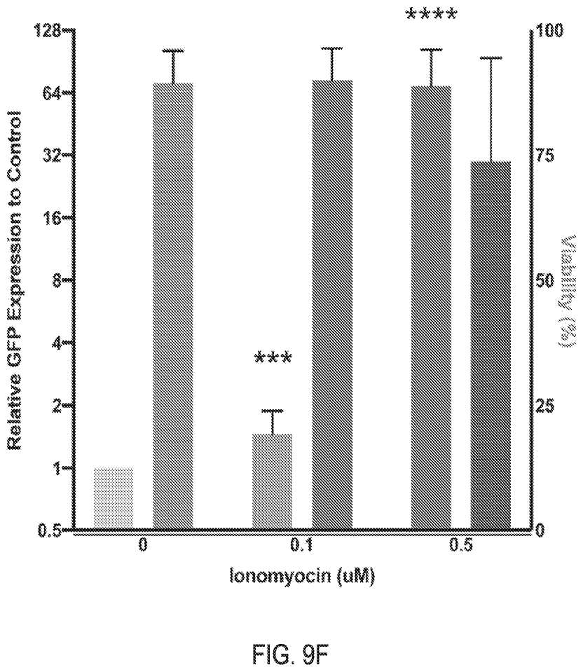

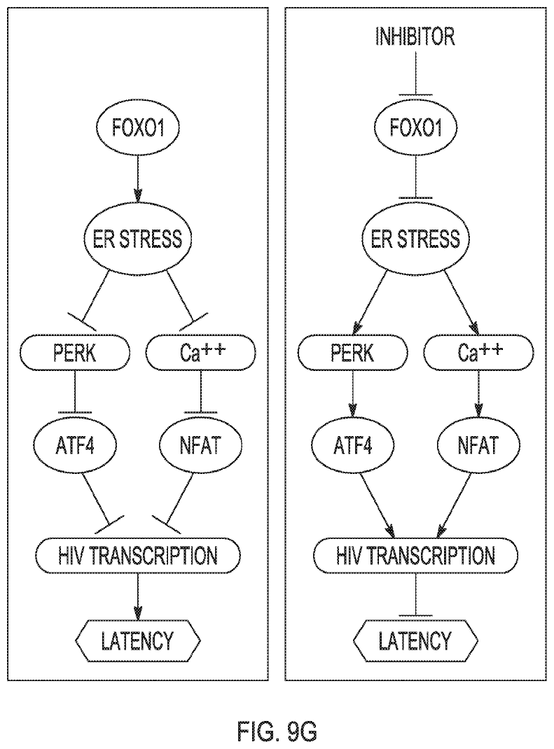

Because ATF4 can be recruited to the HIV LTR (Jiang, G. et al. Downloaded from. MBio 8, 1518-1534 (2018)) and the related human T-cell leukemia virus type 1 (HTLV-1) Reddy, T. et al. Oncogene 14, 2785-2792 (1997)), chromatin immunoprecipitation (ChIP) experiments were performed to test whether ATF4 binds the HIV LTR in response to FOXO1 inhibition. While low levels of ATF4 bound HIV LTR chromatin in untreated J-Lat cells, its recruitment was 3-fold enriched after AS1842856 treatment (FIG. 9, Panel A). A marked increase in RNA polymerase II recruitment to the HIV promoter was observed after FOXO1 inhibition, pointing to possible effects on transcription initiation. In contrast, the RelA subunit of the NF-κB transcription factor, a master regulator of HIV transcription in response to T cell activation or TNFα exposure, was not recruited to the HIV LTR in response to AS1842856 treatment but was significantly enriched after TNFα treatment as expected (FIG. 9, Panel A). The nuclear factor of activated T cells (NFAT) can occupy the NF-κB binding sites in the HIV LTR, and is activated through calcium mobilization after T cell activation rather than protein kinase C activation as for NF-κB. NFAT was markedly enriched at the HIV LTR after FOXO1 inhibition (FIG. 9, Panel A), indicating that FOXO1 in resting T cells suppresses a (non-canonical) transcriptional program that controls HIV transcription in the absence of the NF-κB. Without intending to be bound by any particular theory, this may explain the synergy observed between AS1842856 and TNFα or prostratin as these compounds activate HIV through NF-κB.

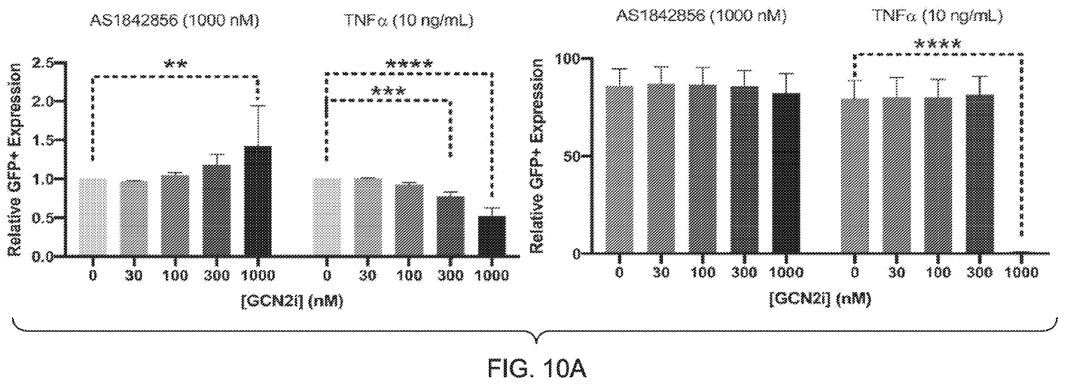

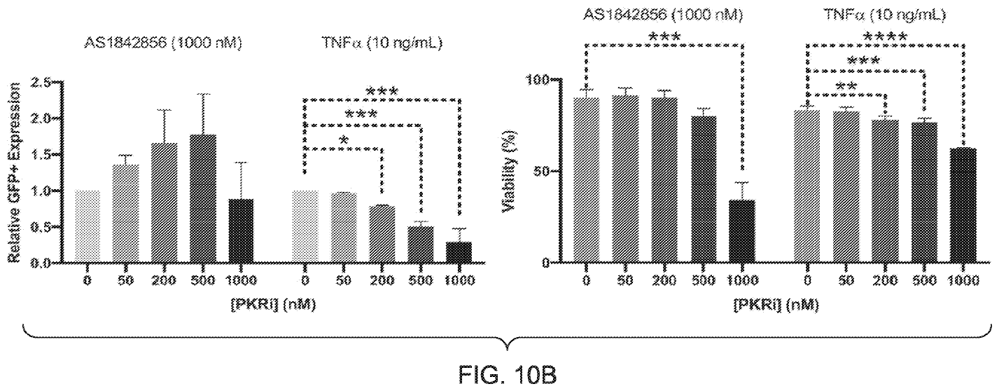

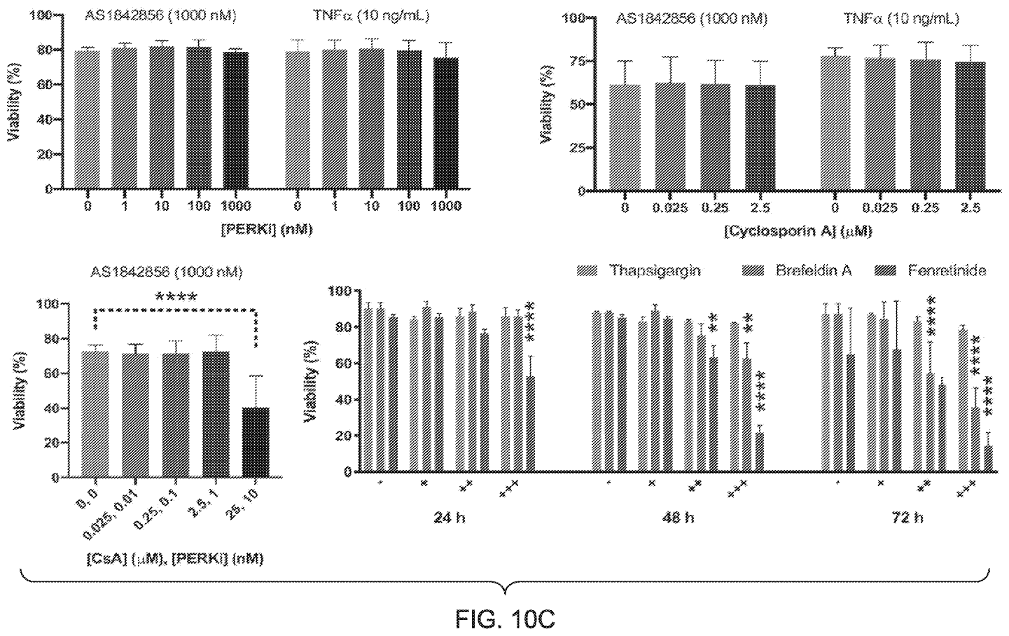

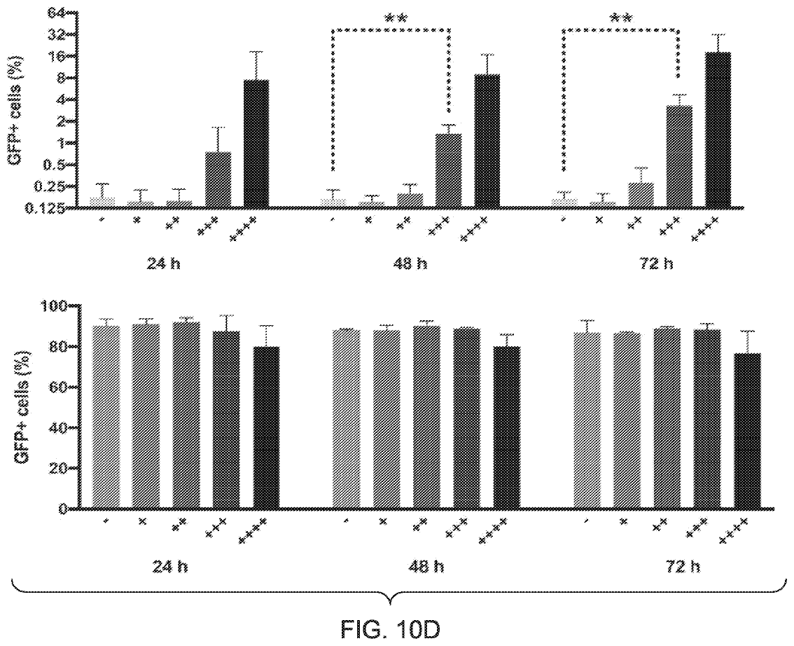

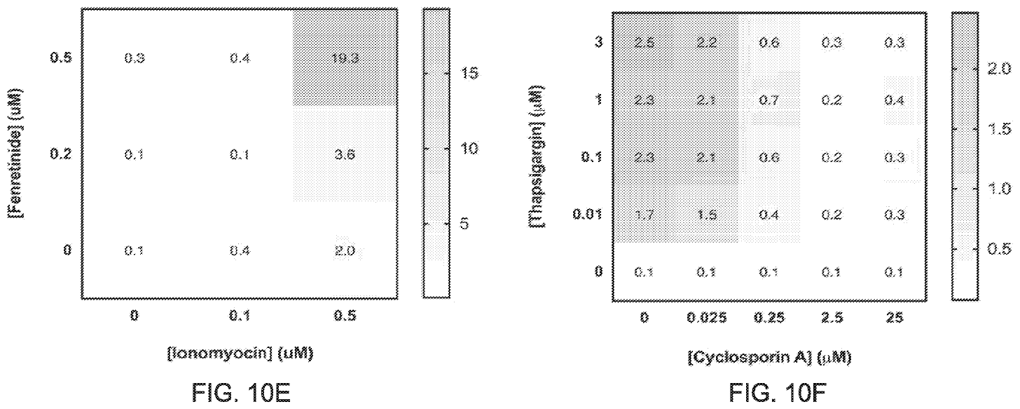

To investigate ISR's potential role in HIV reactivation, the effect of AS1842856 was evaluated in combination with inhibitors of eIF2α kinases: the double-stranded RNA-dependent protein kinase R (PKR) activated by viral infection, the PKR-like endoplasmic reticulum kinase (PERK) activated in response to ER stress, and the general control nondeprepressible 2 (GCN2) activated by amino acid deficiencies. Only treatment with a pharmacological inhibitor of PERK, but not GCN2 or PKR, reduced AS1842856-induced HIV reactivation, consistent with the model that FOXO1 inhibition activates ATF4 through ER stress (FIG. 9, Panel B and FIG. 10, Panel C). In contrast, TNFα-mediated HIV reactivation was not affected by PERK inhibition (FIG. 9, Panel B) but was diminished upon GCN2 or PKR inhibitor treatment (FIG. 10, Panels A & B). This underscores the fundamentally different molecular mechanism underlying latency reversal by the two activators.

Because NFAT was also recruited to the HIV LTR after FOXO1 inhibition, and previous work showed that treatment with cyclosporin A (CsA), a potent inhibitor of calcium release and NFAT mobilization, suppressed HIV reactivation by 50% in primary CD4⁺ T cells (Cron, R. Q. et al. Clin. Immunol. 94, 179-191 (2000)), the effect of CsA was tested on AS1842856-mediated HIV reactivation. Increasing concentrations of CsA suppressed AS1842856-, but not TNFα-induced HIV reactivation, supporting a specific role of NFAT in FOXO1 inhibitor-mediated latency reversal (FIG. 9, Panel C). Treatment with CsA or the PERK inhibitor alone suppressed HIV reactivation by ˜50%, while the combination of both almost completely blocked AS1842856-mediated viral reactivation (˜85%). This supports the model that both ATF4 and NFAT were important for HIV reactivation in response to FOXO1 inhibition.

Both transcription factors have been linked to ER stress, either through activation of PERK or mobilization of ER-resident calcium stores. To test the concept that ER stress reactivates HIV from latency, J-Lat cells were treated with known inducers of ER stress (Corazzari, M. et al. Br. J. Cancer. 96, 1062-1071 (2007)) including thapsigargin, a sarco/ER Ca²⁺ ATPase [SERCA] inhibitor, Brefeldin A, a retrograde vesicular transport inhibitor, and fenretinide, a clinically tested synthetic retinoid derivative. All resulted in HIV-1 reactivation with the best reactivation observed with fenretinide treatment (FIG. 9, Panel E). Activation by these agents were associated with dose-dependent levels of cellular toxicity explained with their known function as potent apoptosis inducers (FIG. 10, Panel C). To minimize toxicity caused by high concentrations of fenretinide, a nontoxic dose of fenretinide (0.5 μM) was combined with increasing amounts of ionomycin to mobilize additional Ca²⁺ ions. The combination significantly increased HIV reactivation (˜64-fold over fenretinide alone) with minimal cellular toxicity (FIG. 9, Panel F and FIG. 10, Panel D). These data were in line with a model where FOXO1 suppressed ER stress in latently infected resting T cells, inhibiting the activation of PERK and release of Ca²⁺, which in turn prevents expression and activation of ATF4 and NFAT, respectively. Inhibition of FOXO1 activity induces ER stress and Ca²⁺ release, mobilizing both transcription factors, which are recruited to the HIV-1 LTR to promote HIV transcription and reverse viral latency (FIG. 9, Panel G).

FIG. 9, Panels A-G, demonstrate that FOXO1 inhibition induces HIV reactivation in the absence of NF-κB recruitment via ATF4 and NFAT. FIG. 9, Panel A, depicts the results of chromatin immunoprecipitation (ChIP) assays with antibodies against Pol III, ATF4, RelA, NFAT and IgG control at the HIV LTR, followed by qPCR using primers specific for HIV-1 LTR Nuc0 or Nuc1. Chromatin was prepared from J-Lat A2 and 5A8 cells, in which the LTR was stimulated by 1000 nM AS1842856 treatment, TNFα or which were left untreated/DMSO. FIG. 9, Panel B, depicts results for a J-Lat cell line A58 that was treated (and pre-treated for 1 hr) with increasing concentrations of GSK2656157 (PERK inhibitor II) in combination with 1000 nM AS1842856 (72 h) or 10 ng/mL TNFα (24 h). HIV-GFP reactivation was analyzed by FACS and relativized to the control condition. FIG. 9, Panel C depicts the same experiment as in FIG. 9, Panel B, but cells were treated with increasing concentrations of Cyclosporin A (CsA) and without 1 hr pre-treatment. FIG. 9, Panel D depicts a similar experiment to that in FIG. 9, Panel B and Panel C, but combining increasing concentrations of GSK2656157 (PERK inhibitor II) and Cyclosporin A (CsA). Data represent average SD of 3-6 independent experiments. FIG. 9, Panel E, shows results for a J-Lat cell line A58 that was treated with increasing concentrations of Thapsigargin (0.01, 0.1, 1 μM), Brefeldin A (0.01, 0.1, 1 μg/mL) and Fenretinide (0.5, 2, 5 μM) for 24, 48 and 72 h. HIV-GFP reactivation was analyzed by FACS. FIG. 9, Panel F depicts results for a J-Lat cell line A58 that was treated with 0.5 μM Fenretinide and increasing concentrations of Ionomycin. HIV-GFP reactivation and cell viability were analyzed by FACS. Data were mean±SD of three independent experiments. FIG. 9, Panel G is a schematic representation of FOXO1 inhibition that leads to ER Stress. Thus, ATF4 activation through PERK and NFAT via cytosolic Ca²⁺ release will promote HIV transcription and will prevent HIV latency. p-value: *p<0.05, **p<0.01, ***p<0.001, ****p<0.0001.

FIG. 10, Panels A-F depict results of a J-Lat cell line 5A8 treated with different concentrations of inhibitors. FIG. 10, Panel A, depicts results for a J-Lat cell line A58 that was treated with increasing concentrations of A-92 (GCN2 inhibitor) in combination with 1000 nM AS1842856 (72 h) or 10 ng/mL TNFα (24 h). HIV-GFP reactivation was analyzed by FACS and relativized to the control condition. Histogram plots of percent live cells for each drug treatment are shown. FIG. 10, Panel B, depicts the same experiment as in FIG. 10, Panel A, but cells were treated with increasing concentrations of PKR inhibitor (PKRi). Histogram plots representing viability are also shown. Data are represented by mean±SD of three different donors. FIG. 10, Panel C, shows histogram plots of percent live cells for each drug treatment. Increasing concentrations of GSK2656157 (PERK inhibitor II) (top left), Cyclosporin A (CsA) (top right), combined concentrations of GSK2656157 and Cyclosporin A (bottom left) and increasing concentrations of Thapsigargin (0.01, 0.1, 1 μM), Brefeldin A (0.01, 0.1, 1 μg/mL) and Fenretinide (0.5, 2, 5 μM) for 24, 48 and 72 h (bottom right). FIG. 10, Panel D, depicts a J-Lat cell line A58 that was treated with increasing concentrations of Ionomycin (0.01, 0.1, 0.5, 1 μM) for 24, 48 and 72 hr. HIV-GFP reactivation and cell viability were analyzed by FACS. FIG. 10, Panel E, depicts a J-Lat cell line 5A8 that was treated for 72 h with increasing concentrations of both Fenretinide (Y-axis) and Ionomycin (X-axis) alone or in combination and analyzed by FACS. HIV-GFP reactivation is reported as a percentage of GFP-expressing cells (% GFP+ cells). FIG. 10, Panel F, depicts a J-Lat cell line 5A8 that was also treated for 72 h with increasing concentrations of both Thapsigargin (Y-axis) and Cyclosporin A (X-axis) alone or in combination and analyzed by FACS. HIV-GFP reactivation is again reported as a percentage of GFP-expressing cells (% GFP+ cells). Data represent average f SD of three independent experiments. p-value: *p<0.05, **p<0.01, ***p<0.001, ****p<0.0001.

### REFERENCES

1. Barré-Sinoussi, F., Ross, A. L. & Delfraissy, J.-F. Past, present and future: 30 years of HIV research. Nat. Rev. Microbiol. 11, 877-883 (2013).
2. Archin, N. M., Sung, J. M., Garrido, C., Soriano-Sarabia, N. & Margolis, D. M. Eradicating HIV-1 infection: seeking to clear a persistent pathogen. Nat. Rev. Microbiol. 12, 750-764 (2014).
3. Ruelas, D. S. & Greene, W. C. An Integrated Overview of HIV-1 Latency. Cell 155, 519-529 (2013).
4. Besnard, E. et al. The mTOR Complex Controls HIV Latency. Cell Host Microbe 20, 785-797 (2016).
5. Dahabieh, M. S., Battivelli, E. & Verdin, E. Understanding HIV Latency: The Road to an HIV Cure. Annu. Rev. Med. 66, 407-421 (2015).
6. Spivak, A. M. & Planelles, V. Novel Latency Reversal Agents for HIV-1 Cure. Annu. Rev. Med. 69, 421-436 (2018).
7. Rasmussen, T. A. & Lewin, S. R. Shocking HIV out of hiding: where are we with clinical trials of latency reversing agents? Curr. Opin. HIV AIDS 11, 394-401 (2016).
8. Manning, B. D. & Toker, A. AKT/PKB Signaling: Navigating the Network. Cell 169, 381-405 (2017).
9. Hedrick, S. M., Michelini, R. H., Doedens, A. L., Goldrath, A. W. & Stone, E. L. FOXO transcription factors throughout T cell biology. Nat. Rev. Immunol. 12, 649-661 (2012).
10. Webb, A. E., Kundaje, A. & Brunet, A. Characterization of the direct targets of FOXO transcription factors throughout evolution. Aging Cell 15, 673-685 (2016).
11. Newton, R. H. et al. Maintenance of CD4 T cell fitness through regulation of Foxo1. Nat. Immunol. 19, 838-848 (2018).
12. Gray, S. M., Amezquita, R. A., Guan, T., Kleinstein, S. H. & Kaech, S. M. Polycomb Repressive Complex 2-Mediated Chromatin Repression Guides Effector CD8+ T Cell Terminal Differentiation and Loss of Multipotency. Immunity 1-13 (2017). doi:10.1016/j.immuni.2017.03.012
13. Delpoux, A. et al. Continuous activity of Foxo1 is required to prevent anergy and maintain the memory state of CD8+ T cells. J. Exp. Med. 215, 575-594 (2018).
14. Jeng, M. Y. et al. Metabolic reprogramming of human CD8+ memory T cells through loss of SIRT1. J. Exp. Med. 215, 51-62 (2018).
15. van Grevenynghe, J. et al. Transcription factor FOXO3a controls the persistence of memory CD4+ T cells during HIV infection. Nat. Med. 14, 266-274 (2008).
16. Oteiza, A. & Mechti, N. FoxO4 negatively controls Tat-mediated HIV-1 transcription through the post-transcriptional suppression of Tat encoding mRNA. J. Gen. Virol. 98, 1864-78 (2017).
17. Trinité, B. et al. Suppression of Foxo1 activity and down-modulation of CD62L (L-selectin) in HIV-1 infected resting CD4 T cells. PLoS One 9, (2014).
18. Battivelli, E. et al. Distinct chromatin functional states correlate with HIV latency reactivation in infected primary CD4+T cells. Elife 7, (2018).
19. Shiota, M. et al. Foxo3a Suppression of Urothelial Cancer Invasiveness through Twist1, Y-Box-Binding Protein 1, and E-Cadherin Regulation. Clin. Cancer Res. 16, 5654-5663 (2010).
20. Jordan, A., Bisgrove, D. & Verdin, E. HIV reproducibly establishes a latent infection after acute infection of T cells in vitro. EMBO J. 22, 1868-1877 (2003).
21. Chavez, L., Calvanese, V. & Verdin, E. HIV Latency Is Established Directly and Early in Both Resting and Activated Primary CD4 T Cells. PLoS Pathog. 11, e1004955 (2015).
22. Shan, L. et al. Transcriptional Reprogramming during Effector-to-Memory Transition Renders CD4+ T Cells Permissive for Latent HIV-1 Infection. Immunity 47, 766-775.e3 (2017).
23. Lassen, K. G., Hebbeler, A. M., Bhattacharyya, D., Lobritz, M. A. & Greene, W. C. A flexible model of HIV-1 latency permitting evaluation of many primary CD4 T-cell reservoirs. PLoS One 7, (2012).
24. Ohoka, N., Yoshii, S., Hattori, T., Onozaki, K. & Hayashi, H. TRB3, a novel ER stress-inducible gene, is induced via ATF4-CHOP pathway and is involved in cell death. EMBO J. 24, 1243-1255 (2005).
25. Deval, C. et al. TRB3 Inhibits the Transcriptional Activation of Stress-regulated Genes by a Negative Feedback on the ATF4 Pathway. J. Biol. Chem. (2007). doi:10.1074/jbc.M611723200
26. Jiang, G. et al. HIV Exploits Antiviral Host Innate GCN2-ATF4 Signaling for Establishing Viral Replication Early in Infection Downloaded from. MBio 8, 1518-1534 (2018).
27. Reddy, T. R., Tang, H., Li, X. & Wong-Staal, F. Functional interaction of the HTLV-1 transactivator Tax with activating transcription factor-4 (ATF4). Oncogene 14, 2785-2792 (1997).
28. Atkins, C. et al. Characterization of a novel PERK kinase inhibitor with antitumor and antiangiogenic activity. Cancer Res. 73, 1993-2002 (2013).
29. Cron, R. Q. et al. NFAT1 Enhances HIV-1 Gene Expression in Primary Human CD4 T Cells. Clin. Immunol. 94, 179-191 (2000).
30. Corazzari, M. et al. Targeting homeostatic mechanisms of endoplasmic reticulum stress to increase susceptibility of cancer cells to fenretinide-induced apoptosis: the role of stress proteins ERdj5 and ERp57. Br. J. Cancer 96, 1062-1071 (2007).
31. Spina, C. A. et al. An In-Depth Comparison of Latent HIV-1 Reactivation in Multiple Cell Model Systems and Resting CD4+ T Cells from Aviremic Patients. PLoS Pathog. 9, 1-15 (2013).
32. Chen, R. et al. The general amino acid control pathway regulates mTOR and autophagy during serum/glutamine starvation. J. Cell Biol. 206, 173-82 (2014).
33. Quirós, P. M. et al. Multi-omics analysis identifies ATF4 as a key regulator of the mitochondrial stress response in mammals. J Cell Biol 216, 2027-2045 (2017).
34. Caselli, E., Benedetti, S., Gentili, V., Grigolato, J. & Di Luca, D. Short Communication: Activating Transcription Factor 4 (ATF4) Promotes HIV Type 1 Activation. AIDS Res. Hum. Retroviruses 28, 907-912 (2012).
35. Kode, A. et al. FoxO1 Protein Cooperates with ATF4 Protein in Osteoblasts to Control Glucose Homeostasis. J. Biol. Chem. 287, 8757-8768 (2012).
36. González-Quiroz, M., Urra, H., Limia, C. M. & Hetz, C. Homeostatic interplay between FoxO proteins and ER proteostasis in cancer and other diseases. Semin. Cancer Biol. 50, 42-52 (2018).
37. Thaxton, J. E. et al. Modulation of Endoplasmic Reticulum Stress Controls CD4+ T-cell Activation and Antitumor Function. Cancer Immunol. Res. 5, 666-675 (2017).
38. So, J.-S. Roles of Endoplasmic Reticulum Stress in Immune Responses. Mol. Cells 41, 705-716 (2018).
39. Kops, G. J. P. L. et al. Forkhead transcription factor FOXO3a protects quiescent cells from oxidative stress. Nature 419, 316-321 (2002).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of reactivating latent human immunodeficiency virus (IV) integrated into the genome of a cell infected with HIV, the method comprising contacting the cell with a FOXO1 inhibitor that reactivates latent HIV integrated into the genome of the cell, wherein the contacting takes place in the absence of an exogenously supplied immunodeficiency virus immunogen.

2. The method of claim 1, wherein the FOXO1 is a polypeptide comprising an amino acid sequence having at least 95% amino acid sequence identity to the amino acid sequence set forth in FIG. 11.

3. The method of claim 1, comprising contacting the cell with at least one second agent that reactivates latent HIV.

4. The method of claim 3, comprising contacting the cell with a synergistically effective amount of the at least one second agent.

5. The method of claim 3, wherein the at least one second agent is a histone deacetylase (HDAC) inhibitor, a protein kinase C (PKC) activator, or a bromodomain inhibitor.

6. The method of claim 3, wherein the at least one second agent is a Smyd2 inhibitor.

7-9. (canceled)

10. The method of claim 1, comprising contacting the cell with an effective amount of antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof.

11-38. (canceled)

39. A method of treating a human immunodeficiency virus (HIV) infection in an individual, the method comprising:
administering to the individual an effective amount of a first active agent, wherein the first active agent is a FOXO1 inhibitor that reactivates latent HIV integrated into the genome of a cell in the individual; and
administering to the individual an effective amount of a second active agent, wherein the second agent comprises an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof, and wherein the administering to the individual of an effective amount of a first active agent does not comprise administering an immunodeficiency virus immunogen to the individual.

40. The method of claim 39, wherein one or both of said administering steps is by a vaginal route of administration, by a rectal route of administration, by an oral route of administration, or by an intravenous route of administration.

41. The method of claim 39, comprising administering to the individual at least one second agent that reactivates latent HIV.

42. The method of claim 41, wherein the at least one second agent is a histone deacetylase (HDAC) inhibitor, a protein kinase C (PKC) activator, or a bromodomain inhibitor.

43-45. (canceled)

46. The method of claim 39, wherein the antiretroviral drug is a nucleoside reverse transcriptase inhibitor selected from the group consisting of Zidovudine, Didanosine, Stavudine, Lamivudine, Abacavir, Tenofovir, Combivir, Trizivir, Emtricitabine, Truvada, Epzicom, and combinations thereof.

47. The method of claim 39, wherein the antiretroviral drug is a non-nucleoside reverse transcriptase inhibitor selected from the group consisting of Nevirapine, Delavirdine, Efavirenz, Etravirine, Rilpivirine, and combinations thereof.

48. The method of claim 39, wherein the antiretroviral drug is a protease inhibitor selected from the group consisting of Saquinavir, Indinavir, Ritonavir, Nelfinavir, Amprenavir, Lopinavir, Atazanavir, Fosamprenavir, Tipranavir, Darunavir, and combinations thereof.

49. The method of claim 39, wherein the antiretroviral drug is an entry inhibitor selected from the group consisting of Enfuvirtide, Maraviroc, and a combination thereof.

50. The method of claim 39, wherein the antiretroviral drug is an integrase inhibitor selected from the group consisting of Raltegravir, Elvitegravir, Dolutegravir, and combinations thereof.

51-52. (canceled)

53. A drug delivery device comprising:
a) a first container comprising a FOXO1 inhibitor that reactivates latent immunodeficiency virus transcription; and
b) a second container comprising an antiretroviral drug, wherein the antiretroviral drug is selected from the group consisting of a nucleoside reverse transcriptase inhibitor, a non-nucleoside reverse transcriptase inhibitor, a protease inhibitor, an entry inhibitor, an HIV integrase inhibitor, and combinations thereof.

54. The device of claim 53, wherein the first and second containers are syringes, vials, or ampules.

55-59. (canceled)

60. The method of claim 1, wherein the FOXO1 inhibitor is a small molecule FOXO1 inhibitor.

61. The method of claim 1, wherein the FOXO1 inhibitor is AS1842856 (5-amino-7-(cyclohexylamino)-1-ethyl-6-fluoro-4-oxo-1,4-dihydro-quinoline-3-carboxylic acid) or a pharmaceutically acceptable salt thereof.

62. The method of claim 1, wherein the FOXO1 inhibitor is an siNA, or a nucleic acid encoding an siNA.

63-71. (canceled)


SEQUENCE LISTING (58 sequences)

SEQ ID NO: 1 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 2-10 - skipped (9 entries)

SEQ ID NO: 11 - protein, 11 aa, HIV-1
YGRKKRRQRRR

SEQ ID NO: 12 - protein, 12 aa, Artificial Sequence
  A synthetic polypeptide
RRQRRTSKLMKR

SEQ ID NO: 13 - protein, 27 aa, Artificial Sequence
  A synthetic polypeptide
GWTLNSAGYLLGKINLKALAALAKKIL

SEQ ID NO: 14 - protein, 33 aa, Artificial Sequence
  A synthetic polypeptide
KALAWEAKLAKALAKALAKHLAKALAKALKCEA

SEQ ID NO: 15 - protein, 16 aa, Artificial Sequence
  A synthetic polypeptide
RQIKIWFQNRRMKWKK

SEQ ID NO: 16 - protein, 9 aa, Artificial Sequence
  A synthetic polypeptide
RKKRRQRRR

SEQ ID NO: 17 - protein, 8 aa, Artificial Sequence
  A synthetic polypeptide
RKKRRQRR

SEQ ID NO: 18 - protein, 11 aa, Artificial Sequence
  A synthetic polypeptide
YARAAARQARA

SEQ ID NO: 19 - protein, 11 aa, Artificial Sequence
  A synthetic polypeptide
THRLPRRRRRR

SEQ ID NO: 20 - protein, 11 aa, Artificial Sequence
  A synthetic polypeptide
GGRRARRRRRR

SEQ ID NO: 21 - DNA, 23 bp, Artificial Sequence
  A synthetic oligonucleotide
cctggaggagaagaggaaagaga

SEQ ID NO: 22 - DNA, 20 bp, Artificial Sequence
  A synthetic oligonucleotide
tttctgctctcaggaagatg

SEQ ID NO: 23 - DNA, 21 bp, Artificial Sequence
  A synthetic oligonucleotide
ccaagactttccgcagctatc

SEQ ID NO: 24 - DNA, 21 bp, Artificial Sequence
  A synthetic oligonucleotide
tcaagtggctccacaacaaca

SEQ ID NO: 25 - DNA, 21 bp, Artificial Sequence
  A synthetic oligonucleotide
cacagcttgagtcgtcagaac

SEQ ID NO: 26 - DNA, 22 bp, Artificial Sequence
  A synthetic oligonucleotide
agaaaggtgaccggatttacca

SEQ ID NO: 27 - DNA, 21 bp, Artificial Sequence
  A synthetic oligonucleotide
atggaggtgttagtggtctgt

SEQ ID NO: 28 - DNA, 21 bp, Artificial Sequence
  A synthetic oligonucleotide
tgccgcactcagtgttgttag

SEQ ID NO: 29 - DNA, 21 bp, Artificial Sequence
  A synthetic oligonucleotide
cacgacaggcttgctgaatga

SEQ ID NO: 30 - DNA, 20 bp, Artificial Sequence
  A synthetic oligonucleotide
gaggacgcggtgtcagaaat

SEQ ID NO: 31 - DNA, 21 bp, Artificial Sequence
  A synthetic oligonucleotide
tgattccctcgcctttcaagc

SEQ ID NO: 32 - DNA, 20 bp, Artificial Sequence
  A synthetic oligonucleotide
ctgccttcaagccggttctt

SEQ ID NO: 33 - DNA, 21 bp, Artificial Sequence
  A synthetic oligonucleotide
ggaagggtgatgtgtccaatc

SEQ ID NO: 34 - DNA, 21 bp, Artificial Sequence
  A synthetic oligonucleotide
tgaatgagttagagcccgaga

SEQ ID NO: 35 - DNA, 22 bp, Artificial Sequence
  A synthetic oligonucleotide
tgccaccagactaaactagacg

SEQ ID NO: 36 - DNA, 20 bp, Artificial Sequence
  A synthetic oligonucleotide
aggtcactgcctactgcctg

SEQ ID NO: 37 - DNA, 22 bp, Artificial Sequence
  A synthetic oligonucleotide
atggcaactctaaaggatcagc

SEQ ID NO: 38 - DNA, 23 bp, Artificial Sequence
  A synthetic oligonucleotide
ggatgtgcattctatggtgtacc

SEQ ID NO: 39 - DNA, 25 bp, Artificial Sequence
  A synthetic oligonucleotide
ttgaggacctctgtgtatttgtcaa

SEQ ID NO: 40 - DNA, 20 bp, Artificial Sequence
  A synthetic oligonucleotide
accacaatgtgtctgtcctc

SEQ ID NO: 41 - DNA, 22 bp, Artificial Sequence
  A synthetic oligonucleotide
cgttgaccacggagttaaacag

SEQ ID NO: 42 - DNA, 21 bp, Artificial Sequence
  A synthetic oligonucleotide
gcacgatgcaacggtaaaagc

SEQ ID NO: 43 - DNA, 23 bp, Artificial Sequence
  A synthetic oligonucleotide
cgggatcttgcagtaatgggtat

SEQ ID NO: 44 - DNA, 21 bp, Artificial Sequence
  A synthetic oligonucleotide
ctgctgagagtttagggtcca

SEQ ID NO: 45 - DNA, 23 bp, Artificial Sequence
  A synthetic oligonucleotide
ctgttcctcttggataaagtgct

SEQ ID NO: 46 - DNA, 21 bp, Artificial Sequence
  A synthetic oligonucleotide
gcaattcccgcacaagattct

SEQ ID NO: 47 - DNA, 21 bp, Artificial Sequence
  A synthetic oligonucleotide
cttccgtaaacacgtccagtg

SEQ ID NO: 48 - DNA, 22 bp, Artificial Sequence
  A synthetic oligonucleotide
ggttgctctgctatgagtctct

SEQ ID NO: 49 - DNA, 21 bp, Artificial Sequence
  A synthetic oligonucleotide
tttccggtagaagatgagccc

SEQ ID NO: 50 - DNA, 21 bp, Artificial Sequence
  A synthetic oligonucleotide
tttcctgcatcaagttgtcgt

SEQ ID NO: 51 - DNA, 21 bp, Artificial Sequence
  A synthetic oligonucleotide
taatgccagcacaatgttccc

SEQ ID NO: 52 - DNA, 21 bp, Artificial Sequence
  A synthetic oligonucleotide
gtcttccgccaccttgatctt

SEQ ID NO: 53 - DNA, 19 bp, Artificial Sequence
  A synthetic oligonucleotide
cccgtgcccacaatgagac

SEQ ID NO: 54 - DNA, 21 bp, Artificial Sequence
  A synthetic oligonucleotide
acatcaccacgcaggttacat

SEQ ID NO: 55 - DNA, 22 bp, Artificial Sequence
  A synthetic oligonucleotide
ccaaccccaacaactgtaatct

SEQ ID NO: 56 - DNA, 23 bp, Artificial Sequence
  A synthetic oligonucleotide
tttcgggattgcttatctcagac

SEQ ID NO: 57 - DNA, 20 bp, Artificial Sequence
  A synthetic oligonucleotide
agtgtgtgcccgtctgttgt

SEQ ID NO: 58 - DNA, 20 bp, Artificial Sequence
  A synthetic oligonucleotide
ttcgctttcaggtccctgtt